(19) United States
(12) Patent Application Publication
(10) Pub. No.: US 2025/0333482 A1
(43) Pub. Date: Oct. 30, 2025

(54) MODULATORS OF MESOTHELIAL ECM MOVEMENT

(71) Applicant: Helmholtz Zentrum München Deutsches Forschungszentrum Für Gesundheit Und Umwelt (GmbH), Neuherberg (DE)

(72) Inventors: Adrian FISCHER, München (DE); Martin MÜCK-HÄUSL, Grasbrunn (DE); Yuval RINKEVICH, München (DE); Safouane KADRI, Neuherberg (DE)

(21) Appl. No.: 18/706,612

(22) PCT Filed: Nov. 7, 2022
(86) PCT No.: PCT/EP2022/080892

§ 371 Date: May 1, 2024

(30) Foreign Application Priority Data
Nov. 5, 2021 (EP) 21206688.0

(51) Int. Cl. C07K 14/78; A61K 38/00; C07K 14/495; C12N 9/64; C12N 15/86

(52) U.S. Cl. CPC C07K 14/78; C07K 14/495; C12N 9/6472; C12N 15/86; A61K 38/00; C07K 2319/43; C12N 2750/14143; C12Y 304/22001

(57) ABSTRACT

The present invention relates to a compound for use in a method for the modulation of movement of extracellular matrix (ECM) produced by mesothelial cells forming the surface of an internal organ, towards a site of injury of said organ of a subject suffering from or being at a risk of an injury of said organ. Additionally, the present invention relates to a compound for use in a particular in vivo screening method for identifying a modulator of movement of extracellular matrix (ECM) produced by mesothelial cells towards a site of injury of an internal organ of a subject. Further, the present invention relates to a specific in vitro screening method for identifying a modulator of the movement of ECM towards an external stimulus in a single cell suspension derived from the mesothelium.

### CROSS-REFERENCE TO RELATED APPLICATIONS

The present application claims the benefit of priority of EP Patent Application No. 21 206 688.0 filed 5 Nov. 2021, the content of which is hereby incorporated by reference in its entirety for all purposes.

### TECHNICAL FIELD OF THE INVENTION

The present invention relates to a compound for use in a method for the modulation of movement of extracellular matrix (ECM) produced by mesothelial cells forming the surface of an internal organ, towards a site of injury of said organ of a subject suffering from or being at a risk of an injury of said organ, wherein modulation is inhibition or promotion. Additionally, the present invention comprises a compound for use in a particular in vivo screening method for identifying a modulator of the movement of extracellular matrix (ECM) produced by mesothelial cells towards a site of injury of an internal organ of a subject. Further, the present invention relates to a specific in vitro screening method for identifying a modulator of the movement of ECM towards an external stimulus in a single cell suspension derived from the mesothelium.

### BACKGROUND OF THE INVENTION

Injured tissues are replaced by rigid anatomies through accrual of extracellular matrix. These replenished rigid structural and mechanical continuums allow organismal survival. When normal repair fails, the result is either non-healing chronic wounds or aggravated scarring and fibrosis (Eming, S. A., Martin, P. & Tomic-Canic, M. Wound repair and regeneration: Mechanisms, signaling, and translation. Sci. Transl. Med. 6, (2014); Guo, S. & DiPietro, L. A. Critical review in oral biology & medicine: Factors affecting wound healing. J. Dent. Res. 89, 219-229 (2010)). Impaired wounding and excessive scarring are a tremendous burden for patients and for the global healthcare system, costing tens of billions of dollars per year, just in the US (Nussbaum, S. R. et al. An Economic Evaluation of the Impact, Cost, and Medicare Policy Implications of Chronic Nonhealing Wounds. Value Heal. 21, 27-32 (2018); Shetty, A. & Syn, W. Health and Economic Burden of Nonalcoholic Fatty Liver Disease in the United States and Its Impact on Veterans. 14-19 (2019)).

Half of all deaths in the industrialized world result from kidney, liver, heart or lung fibrosis and lung infection in Covid-19 causes permanent fibrosis (P. Zhang et al. (2020), Bone Res. 8). Fibroproliferative diseases including any kind of fibrosis, scar formation, keloids as seen within organs, and fibrous adhesions, an extra-organ manifestation, also occurs in many other chronic diseases and injuries, and it is the most critical stage that tips the scale towards organ dysfunction, failure, and death.

Although a fibroproliferative disease is thought to initiate by the immune system and inflammation, it is not clear how an injury of an internal organ such as an inflammation (e.g. pneumonia in the lung) causes connective tissue deposition, leading either to forms of fibrosis and scarring or chronic wounds.

Thus, there is a need in the art to investigate the clinical situation behind having an injury of an internal organ of a subject, the ECM movement towards said site of injury and then resulting in a fibroproliferative disease or a chronic wound.

Therefore, the objective of the present invention is to comply with this need.

The solution of the present invention is described in the following, exemplified in the appended examples, illustrated in the figures and reflected in the claims.

### SUMMARY OF THE INVENTION

The present invention deals with a novel mechanism of ECM movement and physical translocation of pre-existing matrix to areas of injury. This was found out by labelling the pre-existing extracellular matrix (ECM) of the surface of different internal organs with N-Hydroxysuccinimide-esters in chemical and viral models of fibrosis, as well as ex-vivo human samples. Basically, the inventors investigated that ECM is moving and that this movement plays a role in a pathogenic state. In particular, it was demonstrated that mesothelial cells are causative for ECM production and movement. In other words, targeting said mesothelial cells which produce the ECM and which form the surface as the outermost lining of each internal organ can modulate the movement of ECM towards a site of injury of said organ of a subject, thus either inhibiting ECM movement or promoting it. Knowing that mesothelial cells produce ECM which then forms with the mesothelial cell the surface of said organ, one can indirectly or directly affect/target said cells, preferably specifically targeting said cells, thus modulating ECM towards a site of injury of said organ of a subject suffering from or being at a risk of an injury of said organ. Even though a small portion of the ECM on the organ surface may also be contributed from other cell types other than mesothelial cells, the mesothelial cells are the main contributor for ECM production and movement of the ECM. This opens a new clinical situation, which has not been defined yet.

Thus, in a first aspect the present invention relates to a compound for use in a method for the modulation of movement of extracellular matrix (ECM) produced by mesothelial cells forming the surface of an internal organ, towards a site of injury of said organ of a subject suffering from or being at a risk of an injury of said organ, wherein modulation is inhibition or promotion.

Additionally, the present invention may also comprise the compound for the use as defined elsewhere herein, wherein modulation comprises that said compound is capable of specifically targeting mesothelial cells.

The present invention may also comprise the compound for the use as defined elsewhere herein, wherein said compound is a transcription construct encoding a gene involved in the modulation of movement of ECM produced by mesothelial cells.

In addition, the present invention may also envisage the compound for the use as defined elsewhere herein, wherein said gene is selected from the group consisting of csta, tgfb, tgfbr2, ctsb, aebp1, col1a1, adamTs1, dcn, sparc, timp1, c1, c2, c3, c4, saa3, hsf1, and dtr or a combination thereof. Additionally or alternatively, the present invention may also envisage the compound for the use as defined elsewhere herein, wherein said gene is selected from the group consisting of mgp, plac8, crip1, lgals1, and ifi2712a or a combination thereof.

Further, the present invention may also encompass the compound for the use as defined elsewhere herein, wherein the transcription construct comprises DNA or RNA.

Further, the present invention may also encompass the compound for the use as defined elsewhere herein, wherein if the transcription construct is a DNA construct, said construct further comprises a mesothelium specific control element and/or promoter element and/or enhancer element. Preferably, the present invention may also encompass the compound for the use as defined elsewhere herein wherein, if the transcription construct is a DNA construct, the mesothelium specific promoter element is any one of a CRIP1, LGALS1, MGP, SAA3 or a SEPP1 promoter, preferably CRIP1.

Additionally, the present invention may also comprise the compound for the use as defined elsewhere herein, wherein if the transcription construct is a DNA construct, said construct further comprises a RNA or protein target sequence.

Further, the present invention may also comprise the compound for the use as defined elsewhere herein, wherein if the transcription construct is a RNA construct, said construct further comprises a RNA or protein target sequence.

Additionally, the present invention may also encompass the compound for the use as defined elsewhere herein, wherein the compound is an agonist or antagonist of a mesothelium specific receptor.

Further, the present invention may also comprise the compound for the use as defined elsewhere herein, wherein the agonist or antagonist is selected from an antibody, a siRNA, a nucleic acid, an aptamer, a peptide, a protein, a lipid, or a small organic molecule.

Additionally, the present invention may also encompass the compound for the use as defined elsewhere herein, wherein the mesothelium specific receptor is selected from the group consisting of MSLN1, GPM6A, PDPN, TGF-β receptor, LTB4 receptor BLT2, Podoplanin, and Procr.

Further, the present invention may also comprise the compound for the use as defined elsewhere herein, wherein the compound is administered via injection or infusion.

Additionally, the present invention may also encompass the compound for the use as defined elsewhere herein, wherein the administration is performed intravenously, intraperitoneally, intrapleurally, intrathecally, via pericardiocentesis or via the lymphatic system.

Further, the present invention may also comprise the compound for the use as defined elsewhere herein, wherein the compound is administered via a viral vector, a liposome, a transfection reagent, an extracellular vesicle or directly.

Further, the present invention may also envisage the compound for the use as defined elsewhere herein, wherein the viral vector is an adeno-associated virus (AAV) vector and/or an adeno-virus (AV) vector.

Additionally, the present invention may also encompass the compound for the use as defined elsewhere herein, wherein the vector, which is an AAV vector or an AV vector comprises a peptide comprising a RGD motif.

Additionally, the present invention may also comprise the compound for the use as defined elsewhere herein, wherein said internal organ is any one of a lung, a kidney, a heart, a liver, a stomach, a bladder, a peritoneum, a brain, a uterus, a spleen, a pancreas or an intestine.

The present invention may also comprise the compound for the use as defined elsewhere herein, wherein if the modulation of ECM movement is inhibition, said injury of said organ is associated with a chronic wound.

The present invention may also comprise the compound for the use as defined elsewhere herein, wherein if the modulation of ECM movement is promotion, said injury of said organ is associated with a fibroproliferative disease.

The present invention may also comprise the compound for the use as defined elsewhere herein, wherein if the modulation of ECM movement is promotion, said injury of said organ is associated with a fibroproliferative disease, which is fibrosis, preferably any one of lung fibrosis, liver fibrosis, kidney fibrosis, cardiac fibrosis, bladder fibrosis, brain fibrosis, uterus fibrosis, spleen fibrosis, pancreas fibrosis or stomach fibrosis.

In a second aspect the present invention relates to a compound for use in a particular in vivo screening method for identifying a modulator of movement of extracellular matrix (ECM) produced by mesothelial cells towards a site of injury of an internal organ of a subject, the method comprising
a) contacting ECM of an internal organ of a subject with a label;
b) introducing to said organ an injury;
c) contacting mesothelial cells, which form the surface of said organ with a compound of interest;
d) determining whether said compound of interest modulates movement of ECM towards a site of injury of said organ using a detection method in comparison to a control subject having ECM of said organ labelled, but not having mesothelial cells of said organ contacted with said compound of interest,
wherein modulation of movement of ECM towards said site of injury of said organ is indicative for said compound of interest to be a modulator of said ECM movement and wherein step b) and step c) can be switched.

Finally, in a third aspect the present invention relates to an in vitro screening method for identifying a modulator of the movement of extracellular matrix (ECM) towards an external stimulus in a single cell suspension derived from the mesothelium, the method comprising
a) contacting said single cell suspension from the mesothelium with an already labeled ECM or placing said single cell suspension from the mesothelium under suitable conditions which allow said cells to produce ECM and then contacting of said produced ECM with a label;
b) exposing said single cells to an external stimulus;
c) contacting said single cells with a compound of interest;
d) determining whether said compound of interest modulates ECM movement towards said external stimulus using a detection method in comparison to a control single cell suspension, wherein said ECM has been labeled, but said single cells not contacted with said compound of interest,
wherein modulation of the movement of ECM towards said external stimulus is indicative for said compound of interest to be a modulator of said ECM movement and wherein step b) and step c) can be switched.

### BRIEF DESCRIPTION OF THE FIGURES

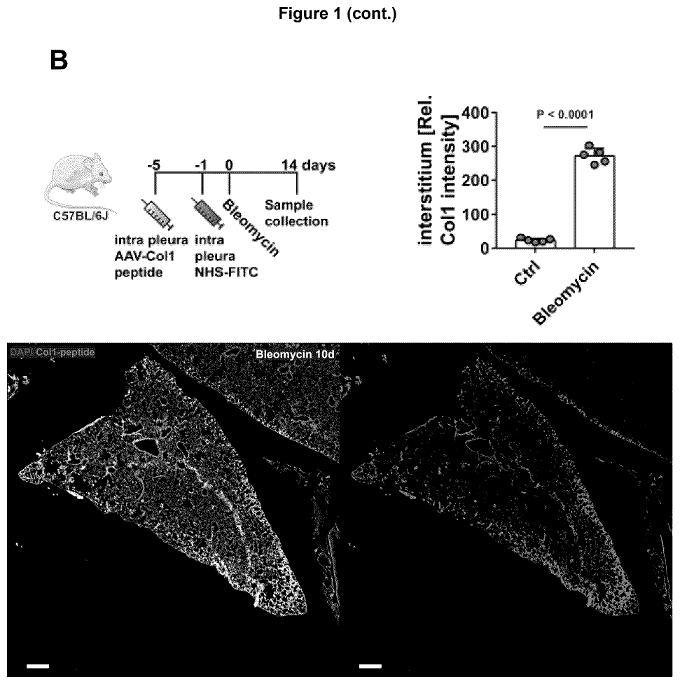

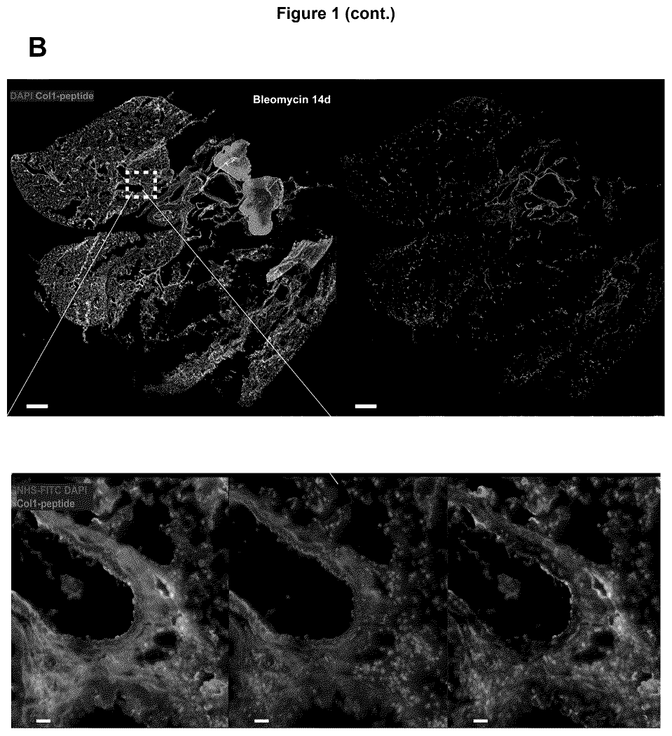

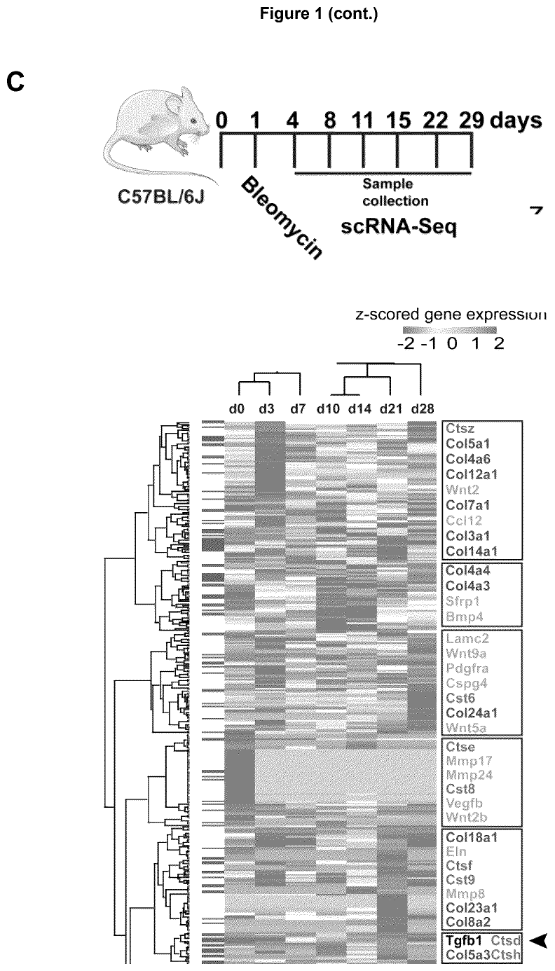

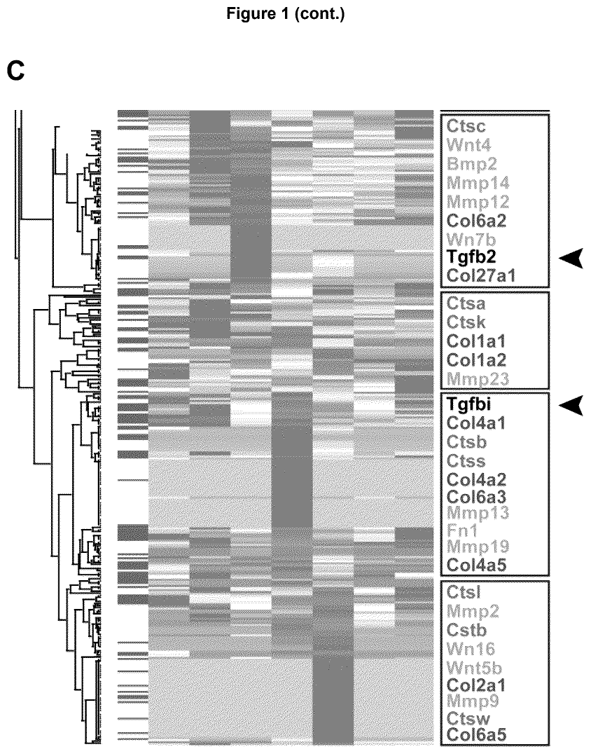

FIG. 1: Mesothelial lining is the source of transferred scar tissue.
(A) Workflow of Col1 binding peptide-based tracing setup. Mesothelial cells transduced by AAV particles express Collagen binding peptides, enabling cell type specific deposition of collagen fibers (n=3). Scale bars: lung surface 100 μM. (B) Mesothelial cells are the source of transferred scar tissue. Lungs were extracted 14 das post bleomycin installation (n=5). Scale bars: lung surface 100 μM, histology overview 1000 μM; 20 μM (visceral pleura and high magnification). (C) Workflow of single cell RNAseq experiment mice sacrificed on days 0, 3, 7, 10, 14, 21 and 28 indicate mesothelial cells have highly dynamic matrix gene expression during the course of bleomycin induced pulmonary fibrosis. Quantification of images. Data represented are mean±SD. One-way ANOVA was used for the multiple comparison (*** P<0.001).

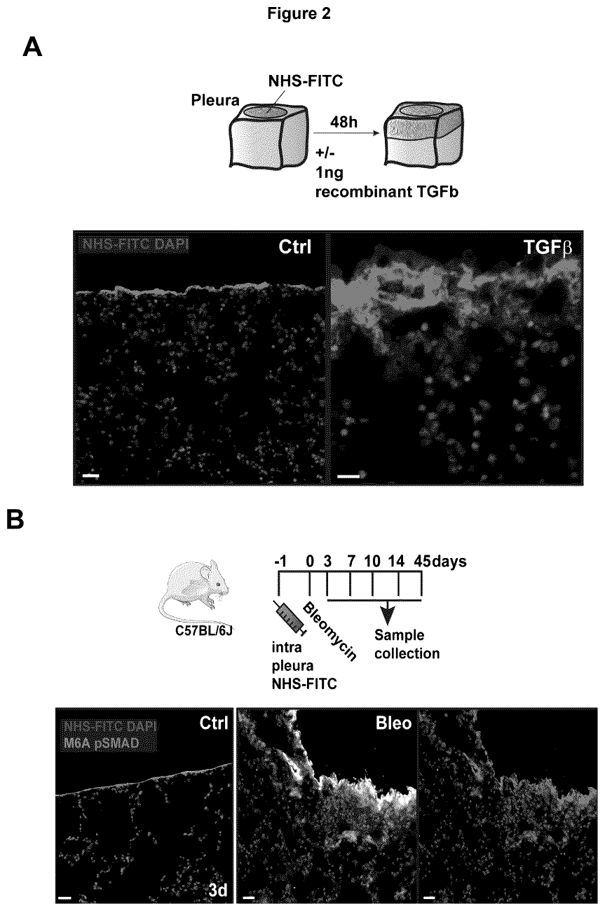

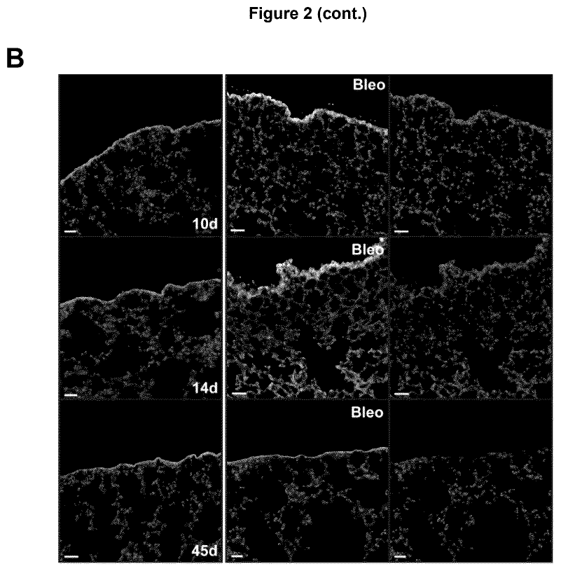

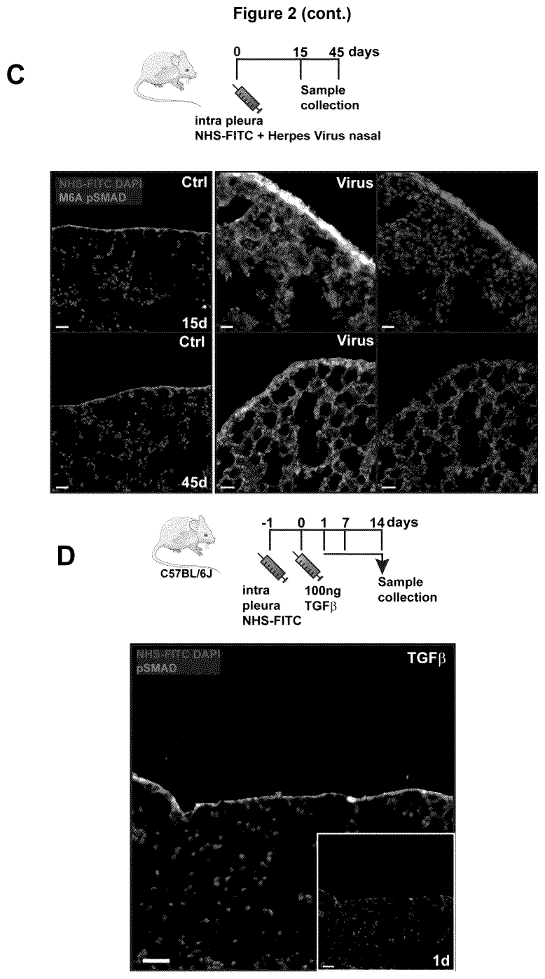

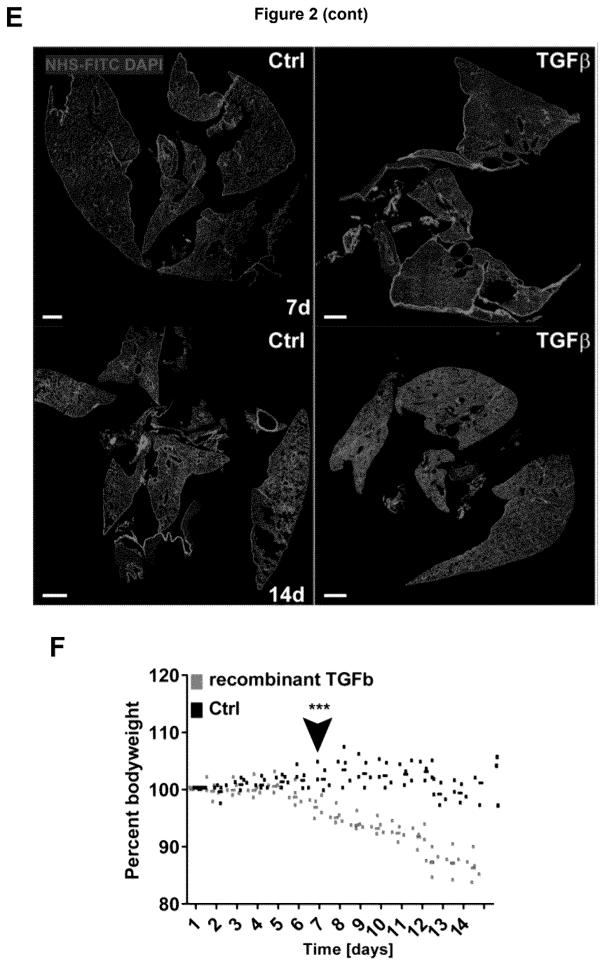

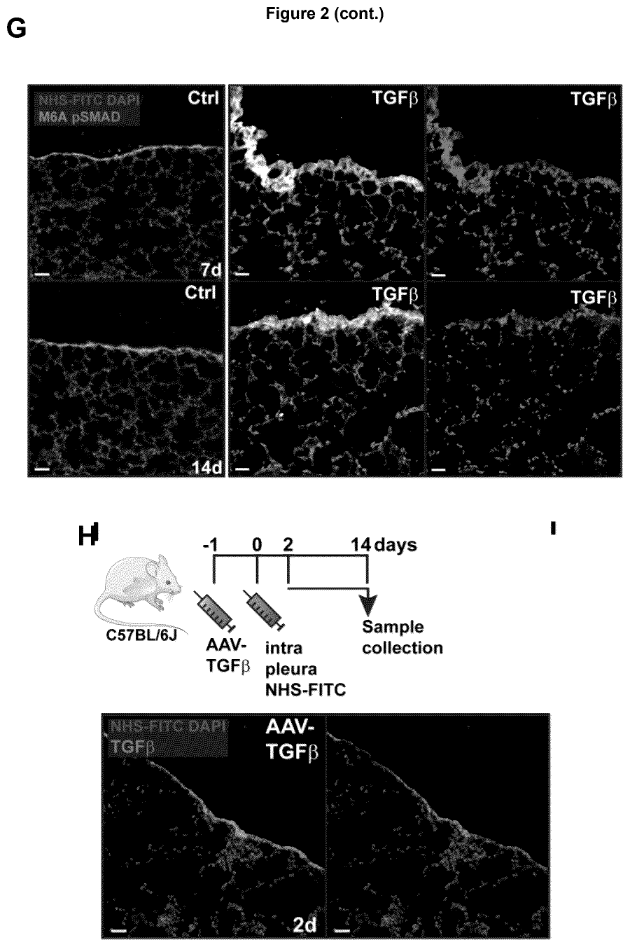

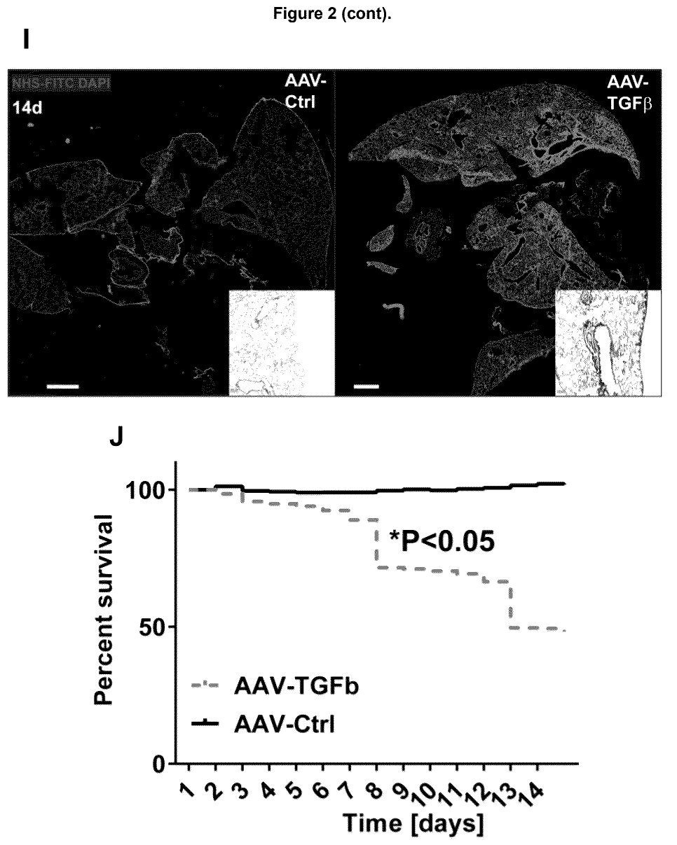

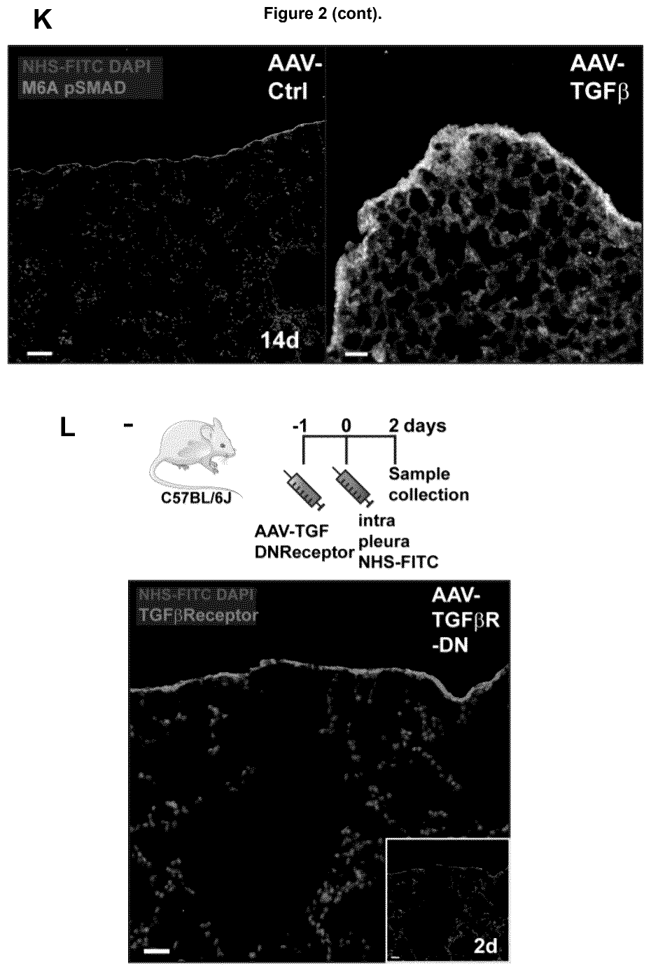

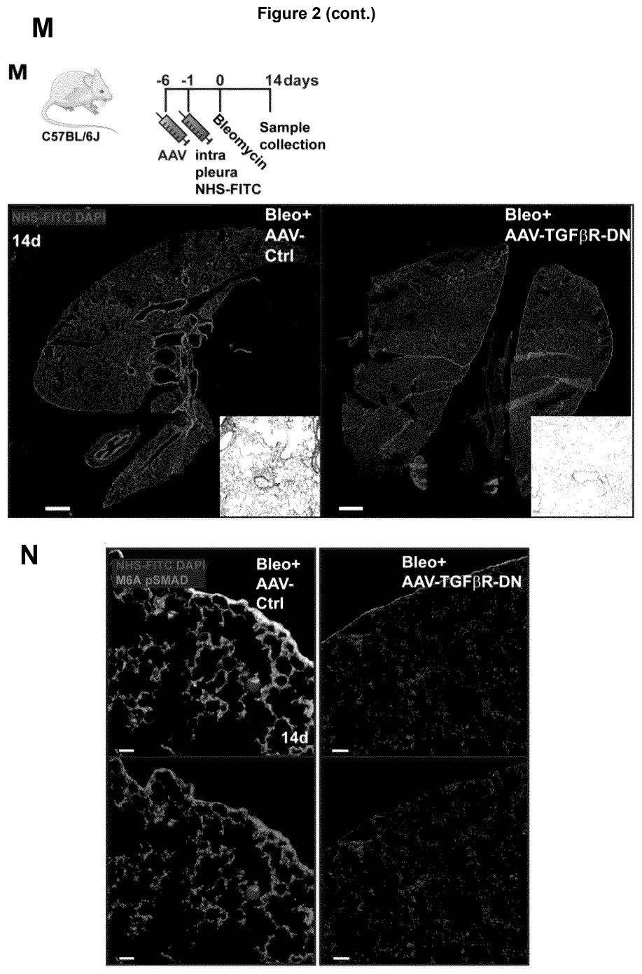

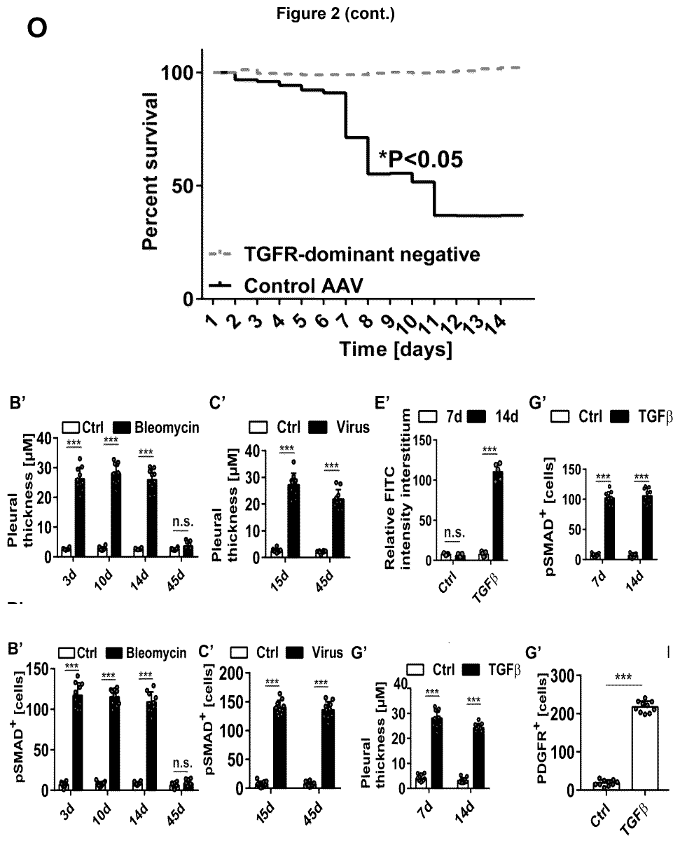

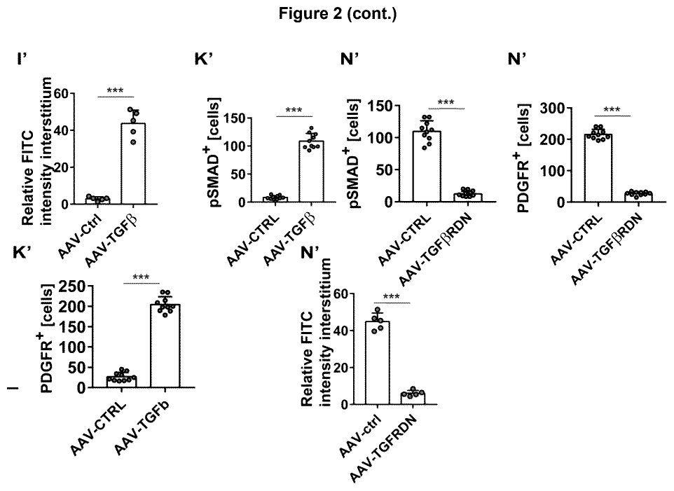

FIG. 2: Pleural scar-accumulation and -invasion are caused by mesothelial TGFβ signaling.
(A) TGFβ induces pleural scar tissue invasion. Lung biopsies were incubated with 1 ng recombinant TGFβ for 48 h (n=3). Scale bars: 20 μM. (B) Pleural matrix invasion in chemically-induced injured lungs, follows TGFβ kinetics in mesothelium. Phosphorylated SMAD (pSMAD) acted as readout for active TGF-β signaling (n>=5). Scale bars: 50 μM. (C) Chronic lung infestation leads to persistent mesothelial TGFβ signaling (n>=3). Scale bars: 50 μM. (D) Workflow of TGFβ induced lung fibrosis model. Mice were intra-plurally injected with NHS-FITC labelling mix. The next day 100 ng of recombinant TGFβ was injected, leading to increased mesothelial TGF-β signaling (n>=5). (E) Active mesothelial TGFβ signaling leads to matrix invasion 14 days post recombinant TGFβ injection (n=5). Scale bars: 1000 μM (overview). (F) TGFβ induced invasion of pleural matrix causes weight loss. (n=5) two-way ANOVA test between day seven: P<0.001. (G) A single injection of recombinant TGFβ induced persistent active mesothelial TGFβ signaling and increased the number of PDGFR+ cells in lung interstitium ((G′)′ (n=5). Scale bars: 50 μM. (H) AAV based particles encoding for active TGF-β increase mesothelial TGF-β levels (n=3). Scale bars: 50 μM. (1) Mesothelial TGFβ activation drives pulmonary fibrosis. AAV particles encoding for active TGF-β were applied intrapleurally (n=5). H&E was used to visualize structural changes. Scale bars: Fluorescence: 1000 μM; H&E: 100 μM. (J) Mesothelial TGFβ activation led to weight loss with increased mortality. (n=5); Log-rank test was used for statistical comparison. (K) Mesothelial TGFβ activation leads to increased interstitial pSMAD levels and PDGFR+ cells (K)′ (n=5). Scale bars: 50 μM. (L) AAV based particles encoding for dominant negative TGF-B receptors express robustly in mesothelial cells (n=3). (M) Targeted inhibition of TGF-β in mesothelial cells blocks bleomycin-induced invasion of pleural matrix. AAV particles encoding for dominant negative TGF-B receptors were applied intrapleurally; five days later bleomycin was installed; 14 days after bleomycin installation organs were harvested (n=5). H&E was used to visualize structural changes. Scale bars: Fluorescence: 1000 μM; H&E: 100 μM. (N) Inhibition of mesothelial TGFβ blocked bleomycin-induced TGFβ signaling and increased the number of PDGFR+ cells in lung interstitium (N)′ (n=5). (O) Inhibition of mesothelial TGFβ prevents bleomycin-induced mortality (n=5). Log-rank test was used for statistical comparison. Quantification of images. Data represented are mean±SD. One-way ANOVA was used for the multiple comparison (***P<0.001).

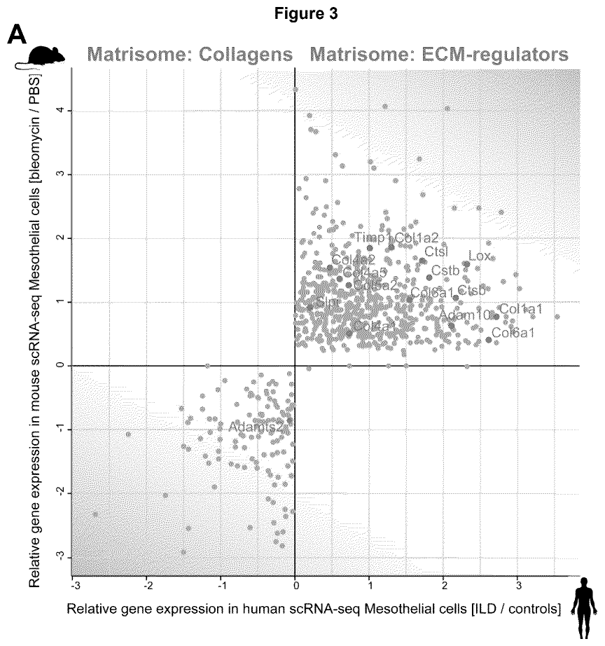

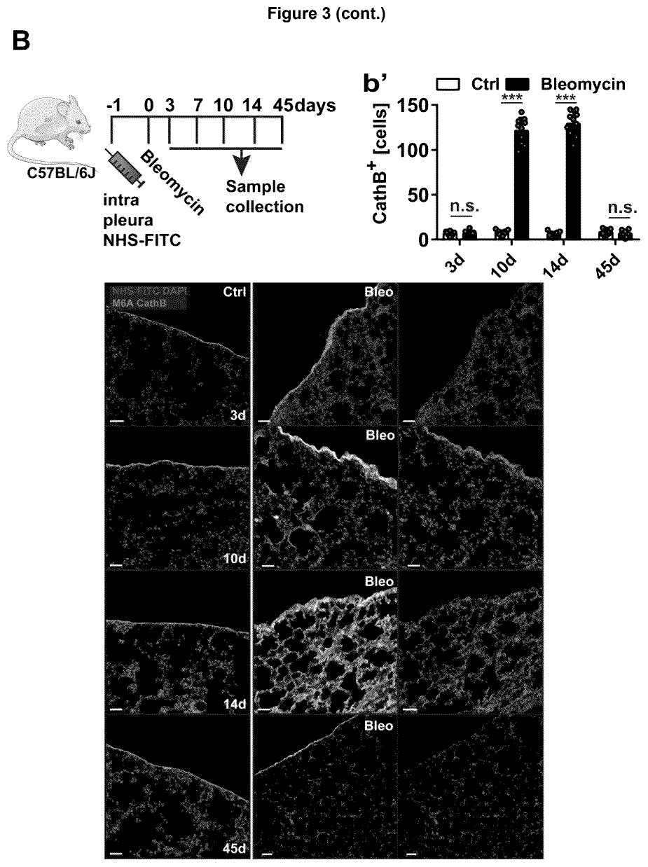

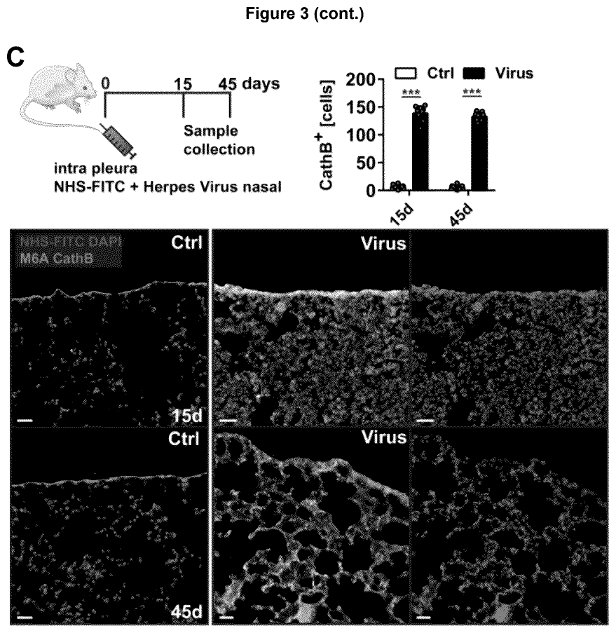

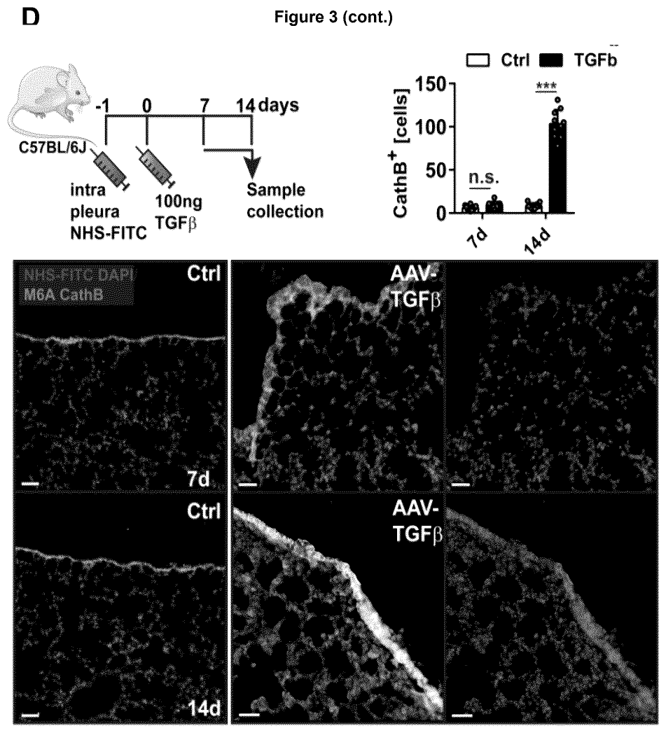

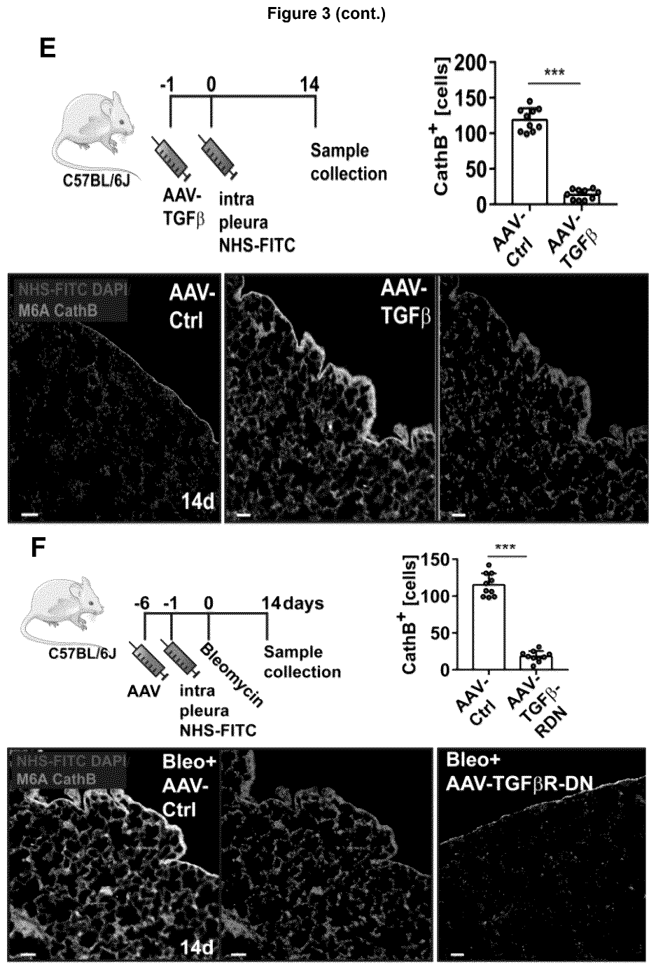

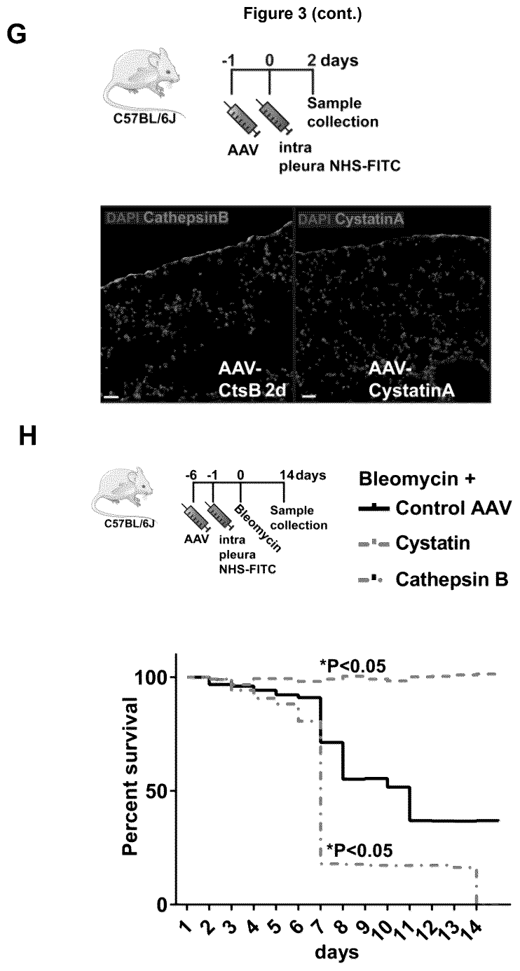

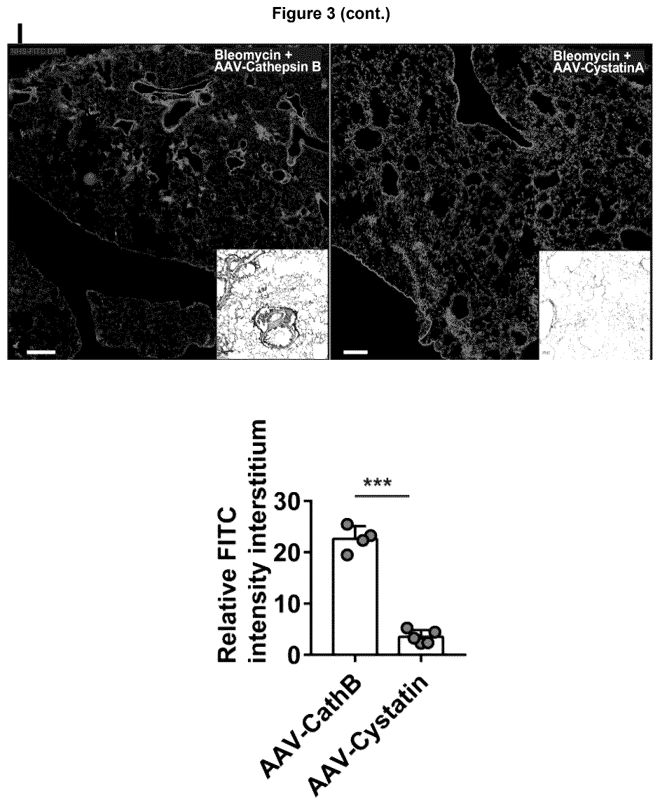

FIG. 3: Mesothelial Cathepsin B releases pleural matrix and aggravates pulmonary fibrosis.
(A) Mesothelial cells from fibrotic human lungs and bleomycin treated animals show increased expression of collagens, cathepsin B and thiol protease mediators. ScRNA-Seq data from bleomycin-installed mice and ILD patients. (B) Cathepsin B expression corresponds to bleomycin-induced pleural fibrosis processes (n>=5). Scale bars: 50 μM. (C) Chronic lung infection leads to persistent mesothelial Cathepsin B expression (n=5). Scale bars: 50 μM. (D) Intrapleurally applied recombinant TGFβ triggers mesothelial Cathepsin B expression after 14 days (n=5). Scale bars: 50 μM. (E) Mesothelial TGFβ signaling leads to increased Cathepsin B levels after 14 days (n=5). Scale bars: 50 μM. (F) Targeted inhibition of TGFβ in mesothelial cells blocks bleomycin induced Cathepsin B expression. AAV particles encoding for dominant negative TGF-B receptors were applied intrapleurally, five days later bleomycin was installed, 14 days after bleomycin installation organs were harvested (n=5). Scale bars: 50 μM. (G) AAV based particles mediate mesothelial specific Cathepsin B or Cathepsin-inhibitor Cystatin overexpression (n=3). Scale bars: 50 μM. (H) Mesothelial Cathepsin activation determines bleomycin-induced mortality (n=5). Log-rank test was used for statistical comparison. (1) Mesothelial Cathepsin B overexpressing lungs of sacrificed animals showed massive matrix invasion seven days after bleomycin installation, while lungs with mesothelial cystatin overexpression were pleural matrix-free two weeks after bleomycin administration (n>=4). H&E was used to visualize structural changes. Scale bars: Fluorescence 1000 μM; H&E 100 μM. Data represented are mean±SD. One-way ANOVA was used for the multiple comparison (***P<0.001).

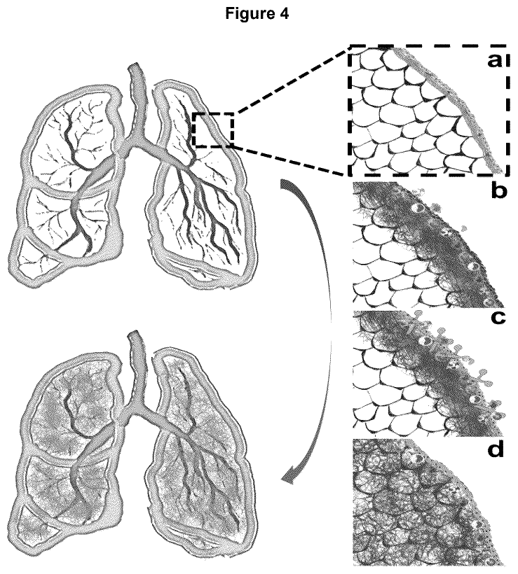

FIG. 4: Model of lung fibrosis development.
(A) A monolayer of mesothelium encapsulates a thin layer of matrix reservoir in healthy lungs. (B) Lung injury recruits inflammatory cells, which activate mesothelial TGFβ signaling, and buildup of pleural matrix reservoirs. (C) Mesothelial Cathepsin B liberates pleural matrix pools, triggering inward invasion and pulmonary fibrosis.

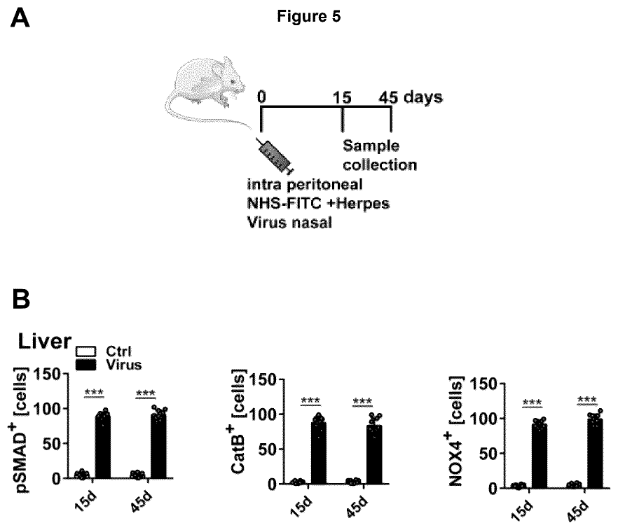

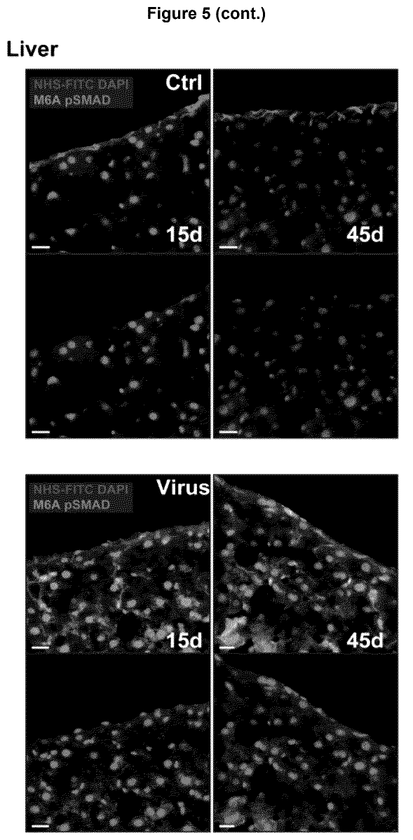

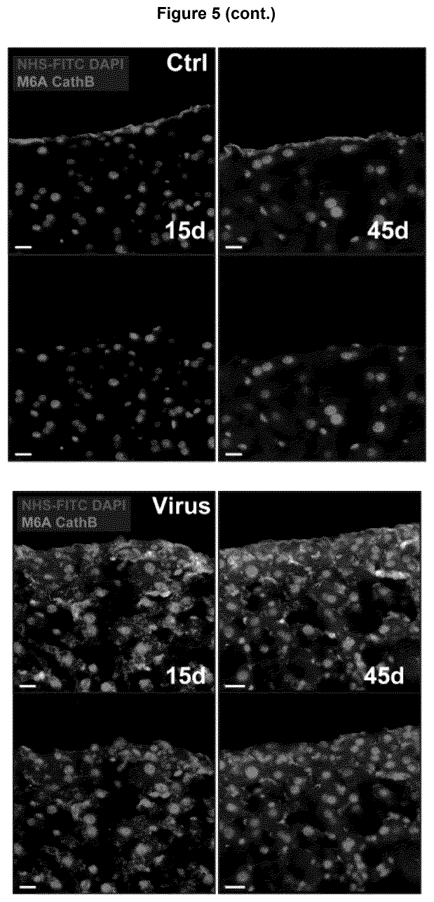

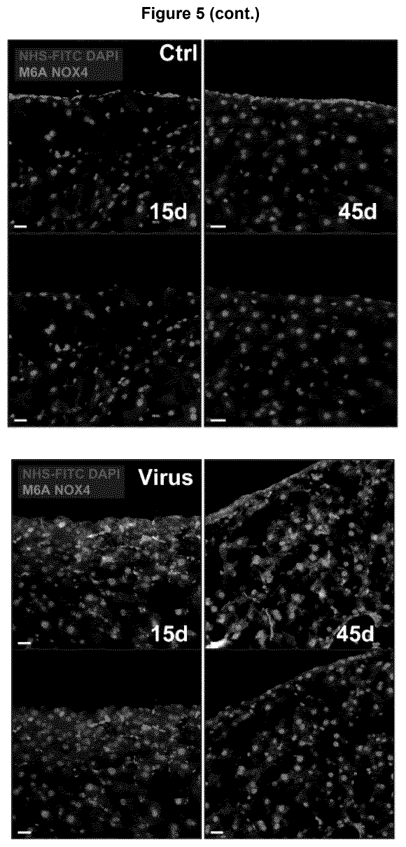

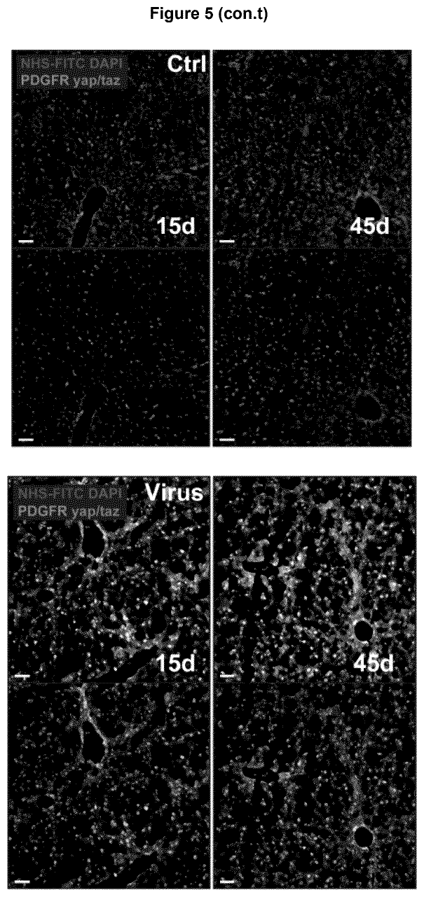

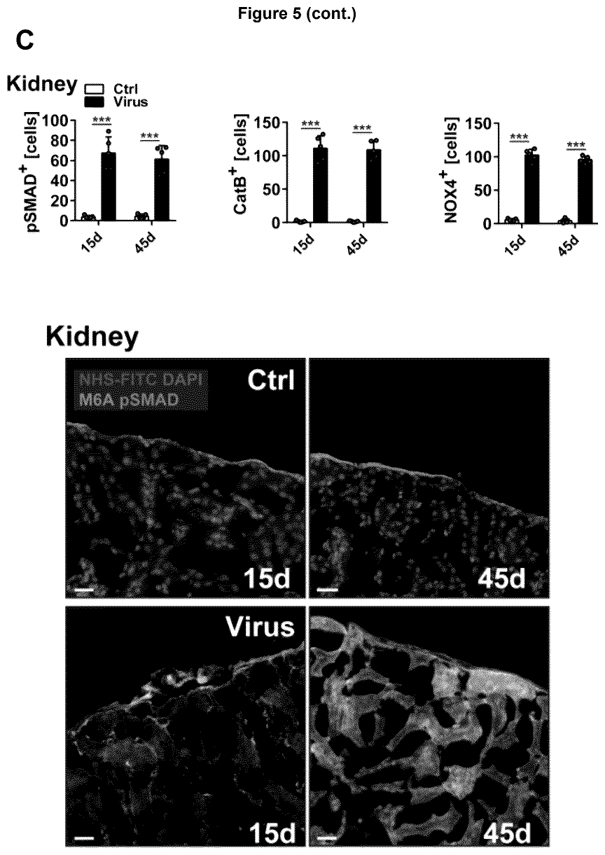

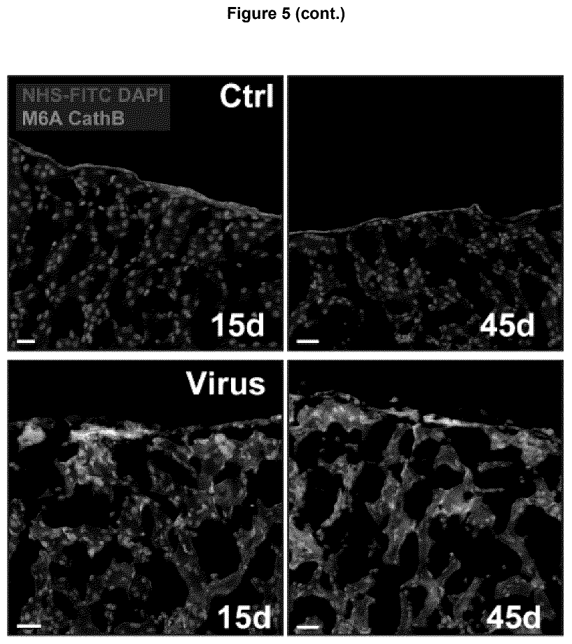

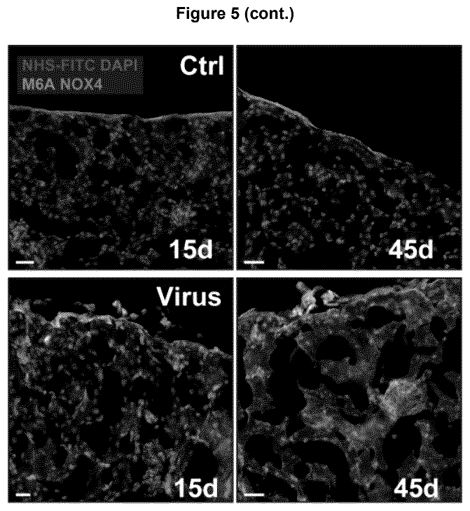

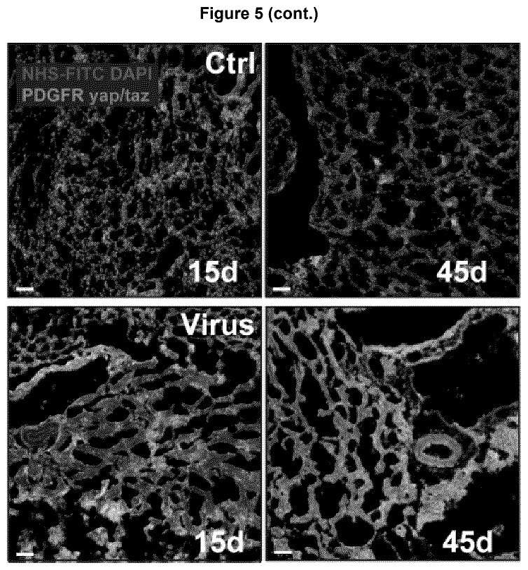

FIG. 5: Liver and kidney of virus administrated mice show lung-like signalling profiles.
(A) Virus installation leads to increased activation of TGFβ signaling, oxidative Stress and Cathepsin. Expression in livers (B) and kidneys (C). Workflow of abdominal matrix fate tracing setup. Mice were intra-peritoneal injected with N-Hydroxysuccinimide-fluorescein isothiocyanate (NHS-FITC) labelling mix and herpes virus was applied intra nasally and sacrificed on day 15 and 45. Histology images of murine livers (n>=1) and kidneys (n>=2). Scale bars: 20 μM. Data represented are mean±SD. One-way ANOVA was used for the multiple comparison (***P<0.001).

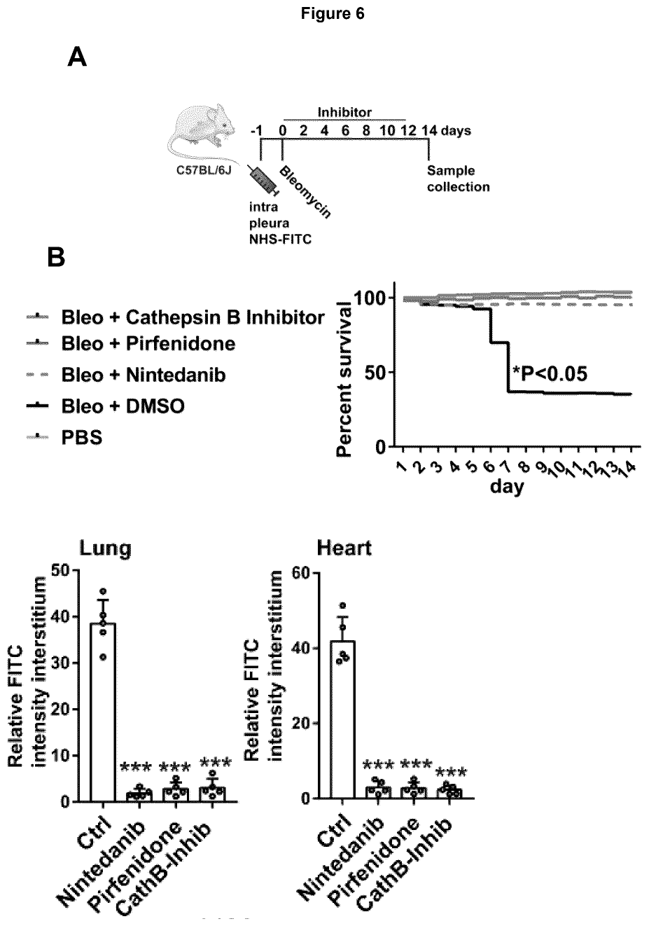

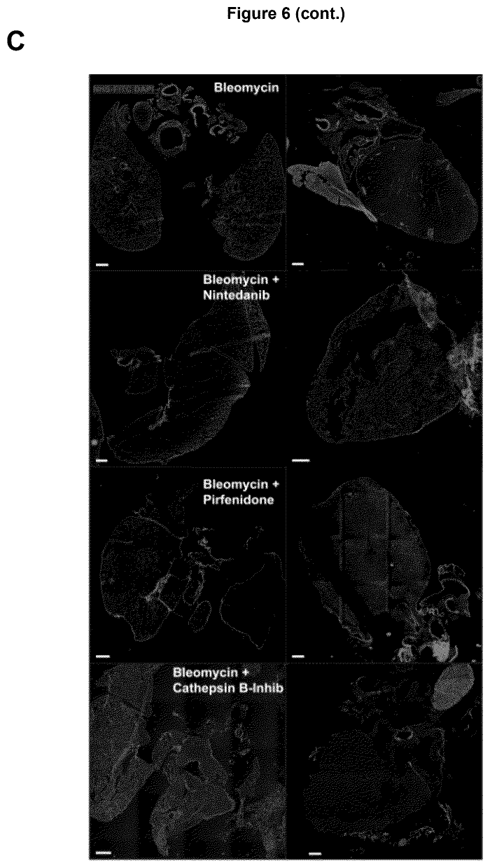

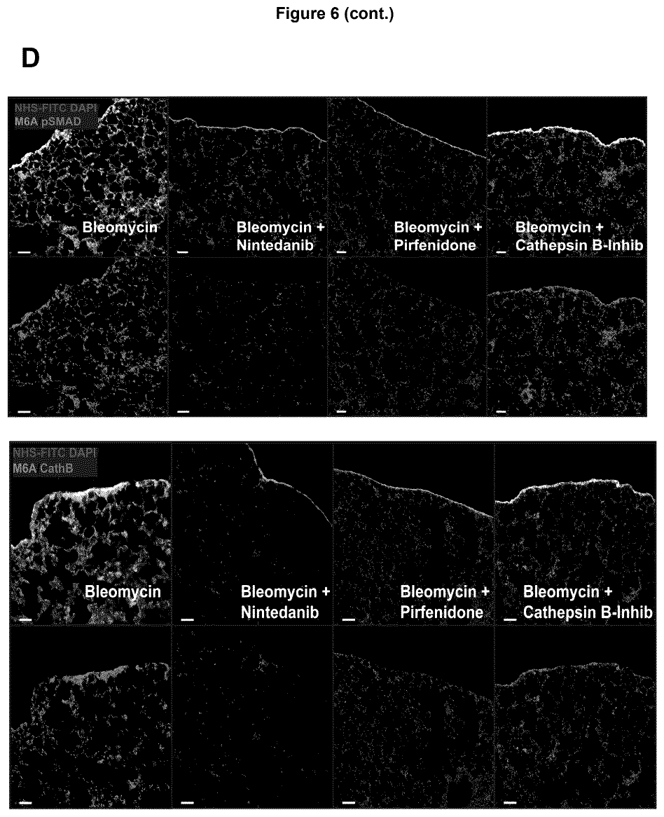

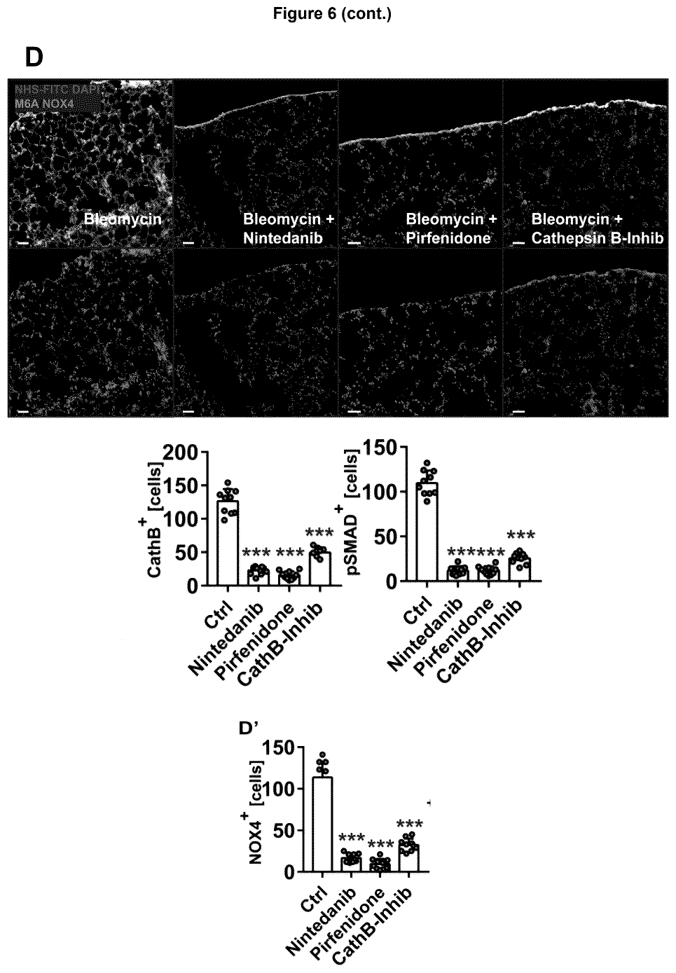

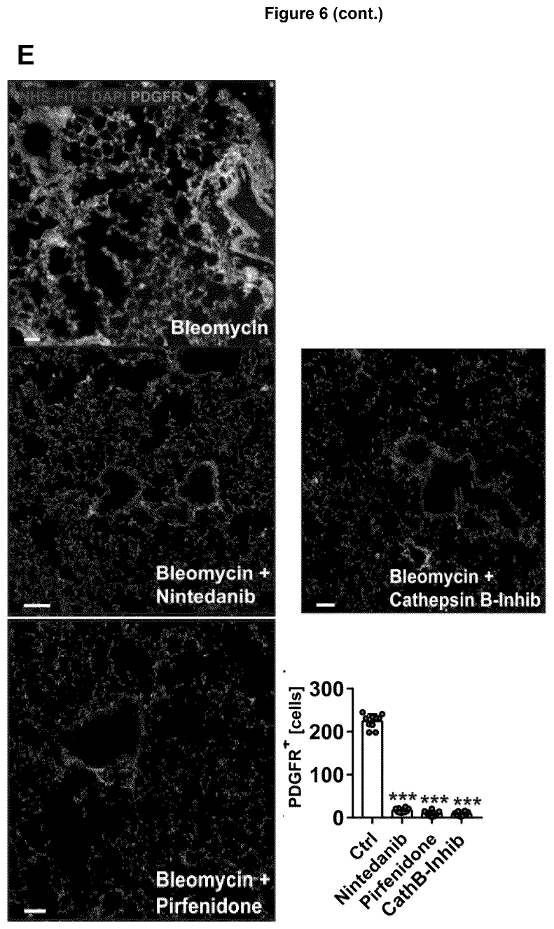

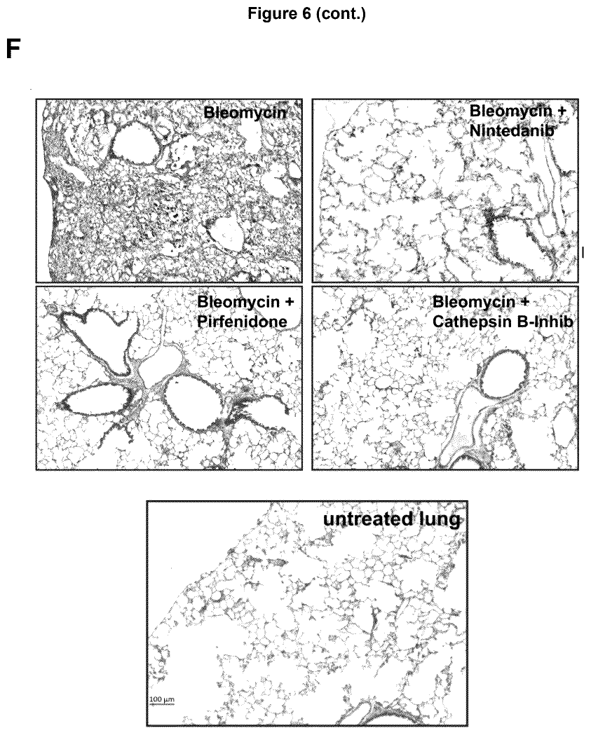

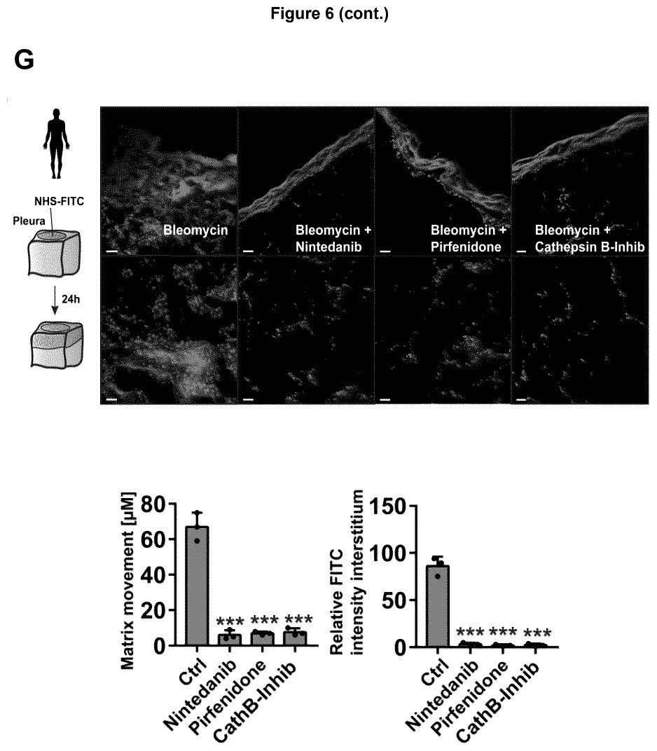

FIG. 6: Ablation of fluid matrix streams prevents pulmonary fibrosis.
(A) Workflow of pharmacologic treatment regime in the bleomycin-induced lung fibrosis model. (B) and (C) Pirfenidone, Nintedanib and Cathepsin B inhibitor prevent mortality and rescue pleural matrix pools after bleomycin-induced injury (n=5). Log-rank test was used for statistical comparison. Scale bars: 1000 μM (Lungs); 500 μM (Hearts). (D-F) Immunofluorescence and H&E images of murine lungs two weeks after bleomycin injury (n=5). Scale bars: 50 μM (Immunostainings); 100 μM (H&E). Quantification of images. Data represented are mean±SD. One-way ANOVA was used for the multiple comparison (***P<0.001). (G) Inhibition of cathepsin B blocks fluid matrix flows in fibrotic human lung tissue. N=3; scale bar: 20 μM. Statistical comparison was performed by unpaired t-test. Data represented are mean±SD.

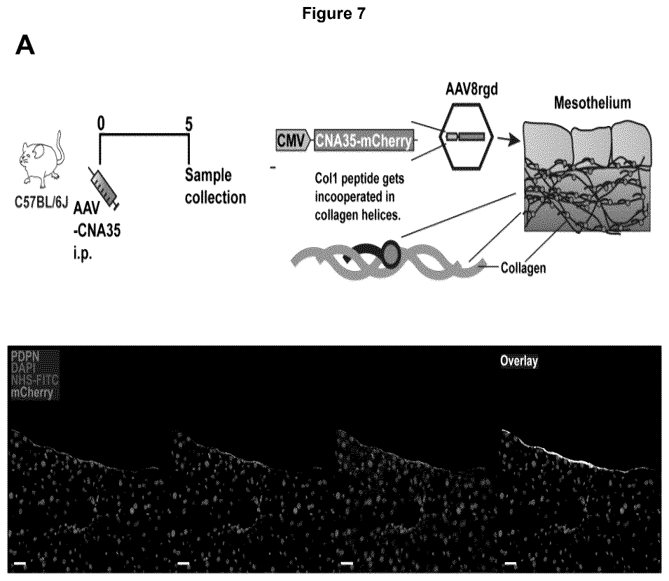

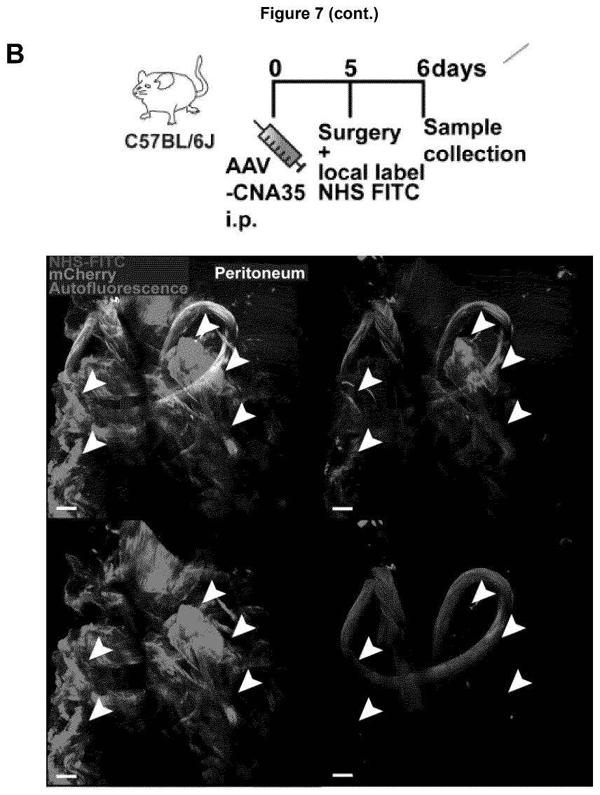

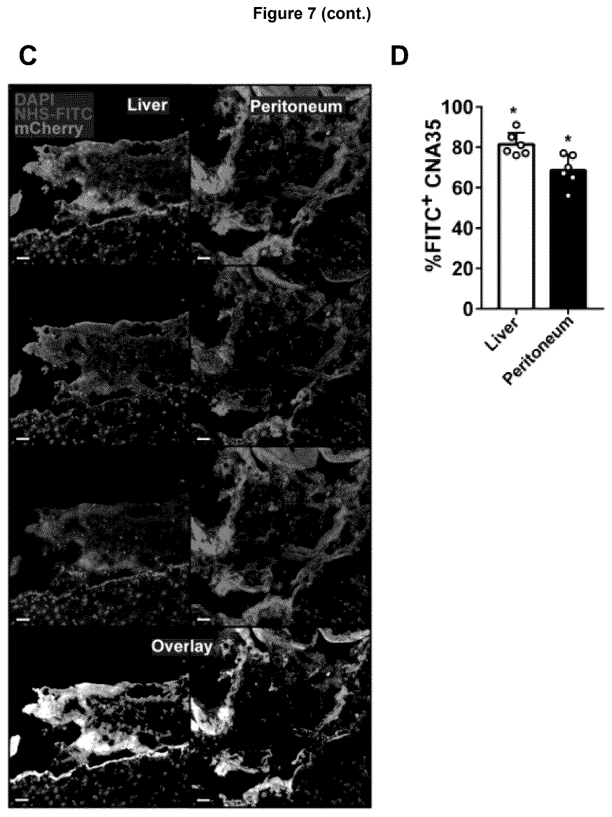

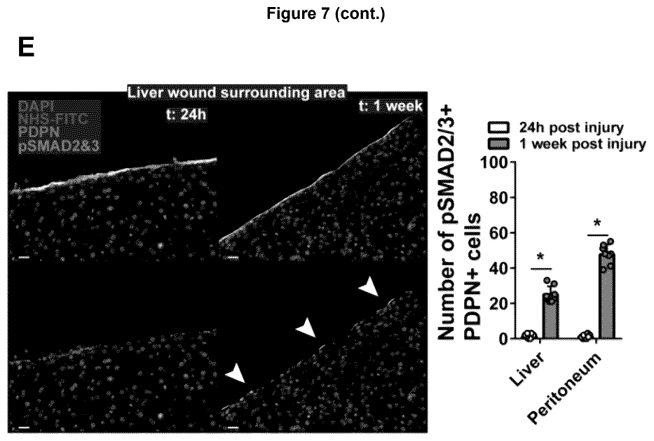

FIG. 7: Mesothelial cells are a source for transferred ECM
(A) Transduced mesothelial cells express CNA35 fused to mCherry, which binds to collagens. Representative immunostaining five days after intra peritoneal injection (B) Laparotomy closure 24 hours post injury of animals transduced with CNA35-mcherry reporter and NHS-FITC surface label. n=five biological replicates. Histology: 500 μm. (C) Wounds of animals transduced with CNA35-mcherry reporter and NHS-FITC surface label 24 hours post injury. n=six biological replicates. Histology: 50 μm. (D) Percentage of FITC+ CNA35+ signal in (C). (E) Mesothelial cells show active pSMAD2/3 signaling 7 days post injury. n=six biological replicates. Histology: 50 μm. Two-tailed Mann-Whitney; *<0.05.

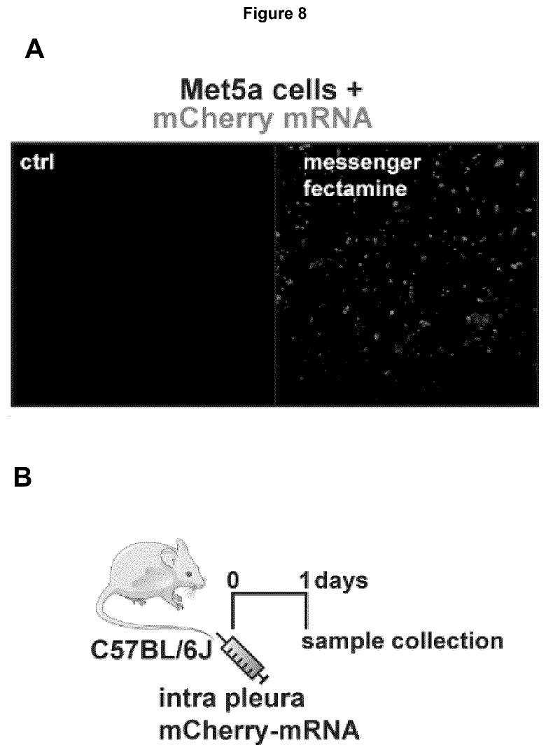

FIG. 8: Robust mRNA mediated transgene expression in vitro and in vivo mesothelium.
(A) Human mesothelial cells were treated once with naked mCherry mRNA (left) and once with mCherry mRNA+Lipofectamine™ MessengerMAX™ (right). Images were taken 24 hours post treatment. n=5. (B) Mice were injected intra pleural with a mCherry mRNA+in vivo-jetPEI® mix, organs were harvested and visualized 24 h after injection. M6A serves as a mesothelial-specific marker.

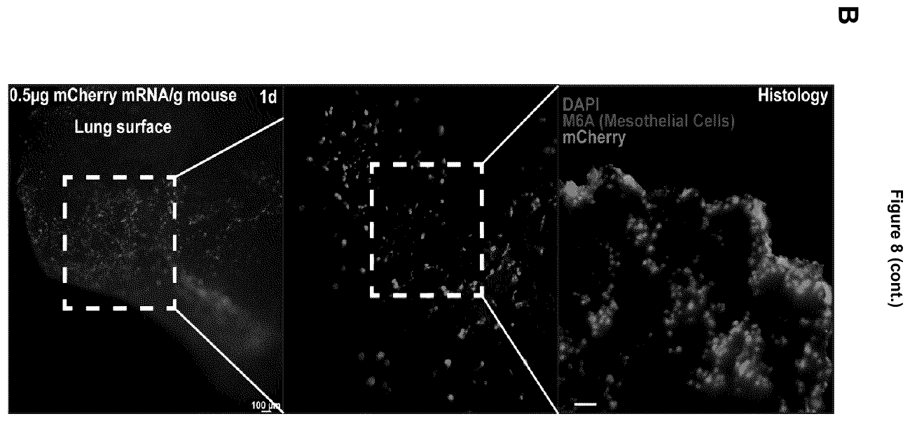

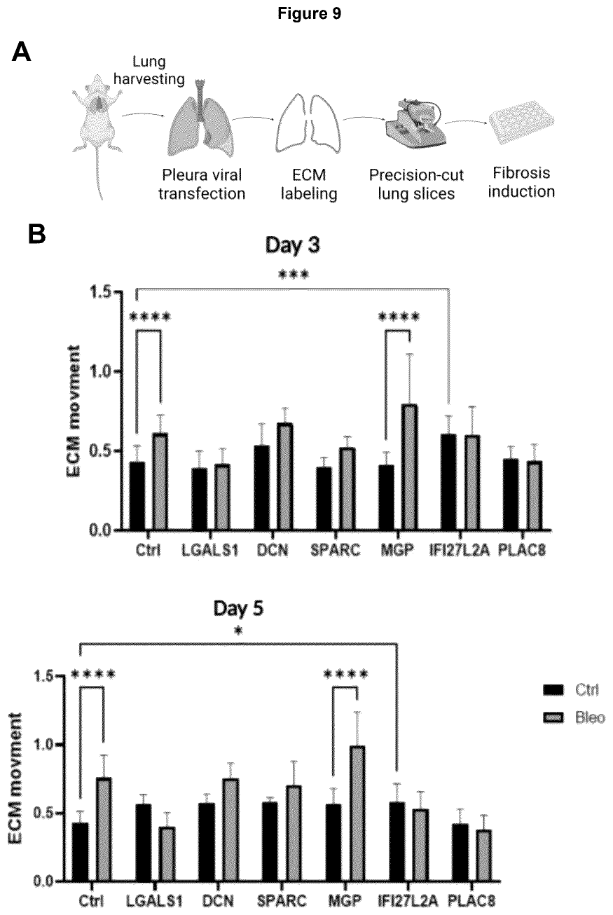

FIG. 9: Further candidate genes for targeted expression in mesothelial cells.
(A) Scheme of PCLS Workflow. Murine lungs are harvested and incubated in adeno associated virus containing solution. Afterwards lungs are incubated in NHS-FITC containing labelling solution. Lungs are cut in thin slices (Precision cut lung slices; PCLS) and incubated in presence of bleomycin or PBS control. After indicated timepoints Surface matrix invasion (movement) is visualized and quantified. (B) Quantification of ECM invasion in PCLS ex vivo assay after 3 and 5 days of incubation. AAV mediated overexpression of IF127L2A induces significant invasion of pleural ECM. Overexpression of LGALS1, PLAC8, DCN and SPARC blocks bleomycin induced ECM invasion significantly. (C) Representative fluorescence images of PCLS 5 days post incubation.

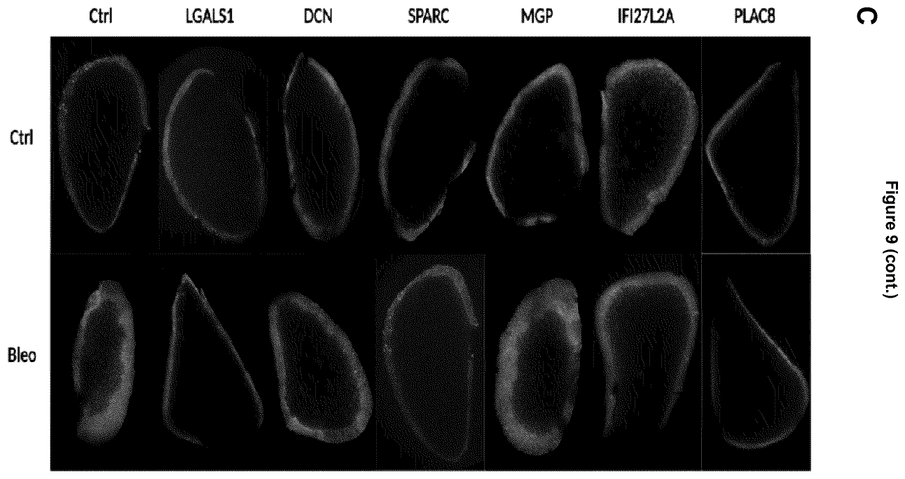

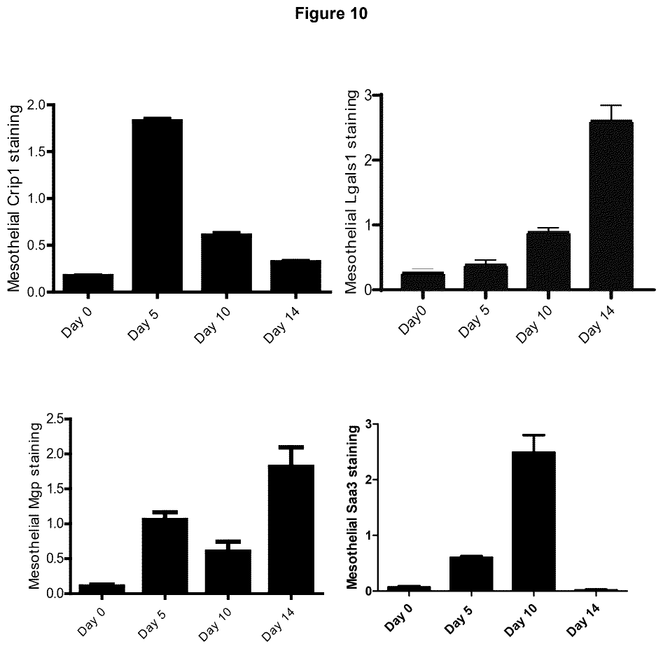

FIG. 10: Mesothelial expression I.
Quantification of mesothelial protein expression of CRIP1, LGALS1, MGP, and SAA3 in mouse lungs 5, 10 and 14 days after intratracheal installation of bleomycin. Those strong protein expressions in mesothelial cells—preferably already at day 5 such as the expression of CRIP1 or MGP—indicate that in such mesothelial cells also the corresponding promoter (e.g. CRIP1 or MGP) is highly activated and which can then be used in the transcription construct as a mesothelium specific promoter element.

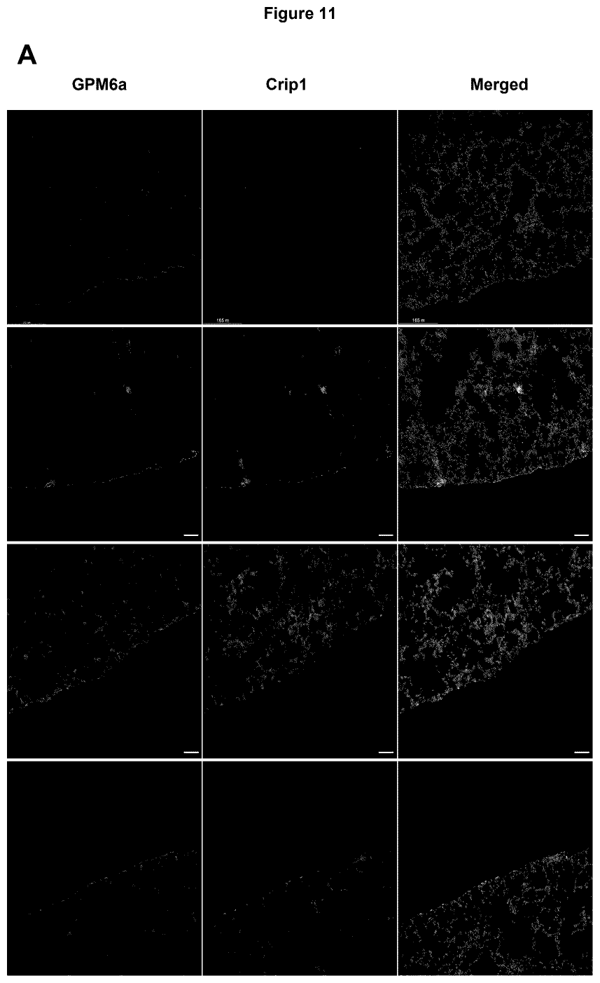

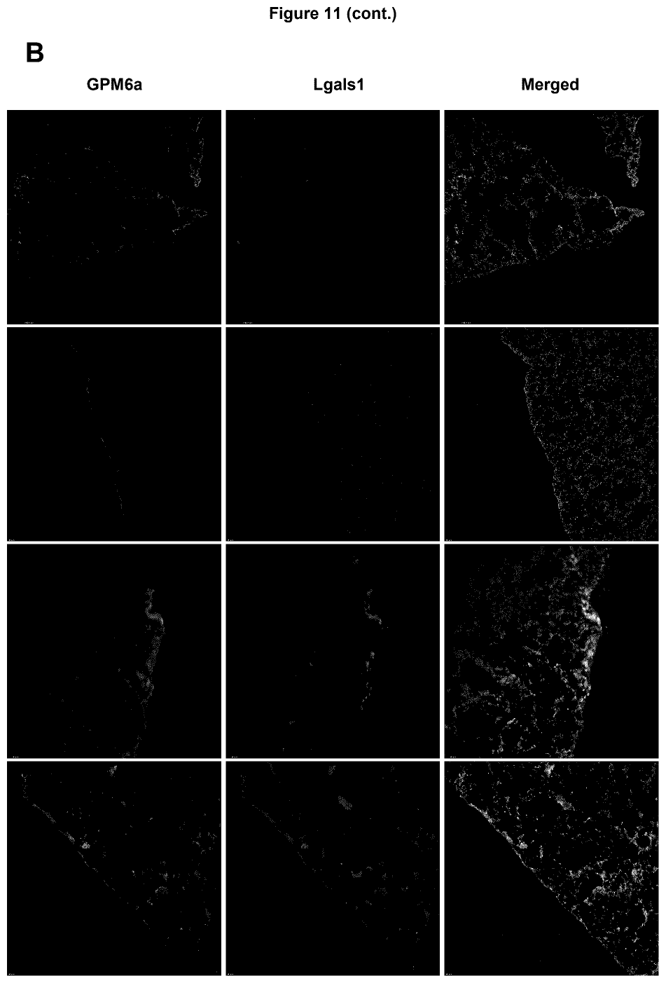

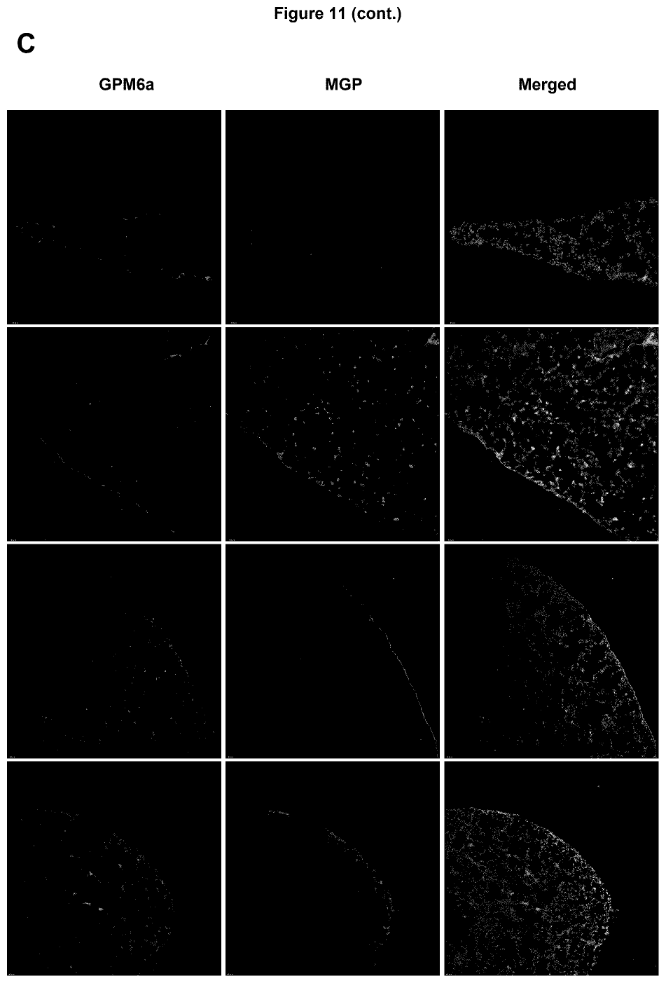

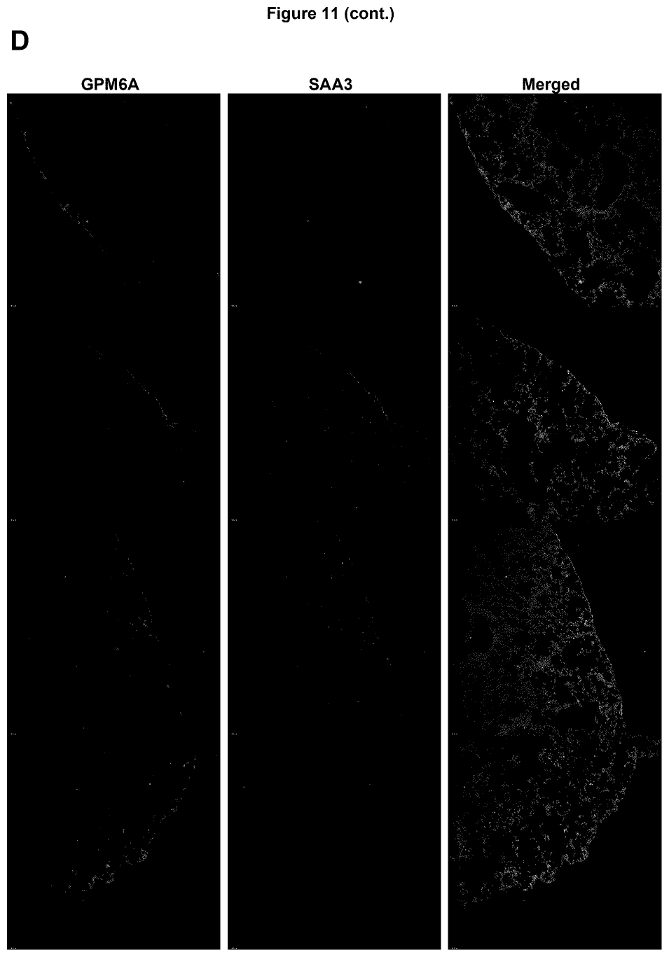

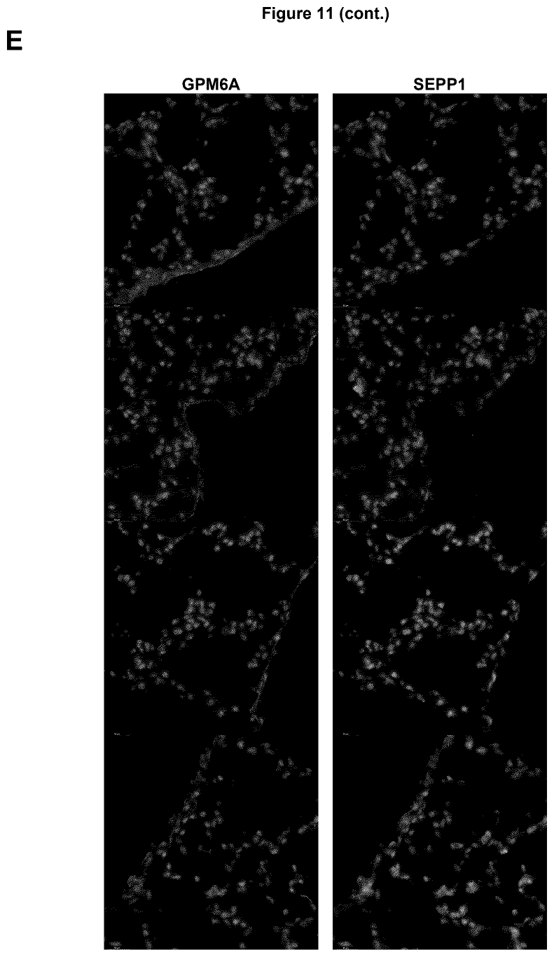

FIG. 11: Mesothelial expression II.
Representative fluorescence staining images of the expression of the mesothelium specific receptor GPM6a alone and then in combination with either (A) CRIP1, (B) LGALS1, (C) MGP, (D) SAA3 or (E) SEPP1.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as one, two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

In order to overcome some of the shortcomings of the means described so far, the inventors provided herein promising new compounds for applying in the modulation of movement of extracellular matrix (short: ECM) which is produced by mesothelial cells which are cells forming the surface of an internal organ in a subject as defined herein.

In sum, the present invention opens a new avenue for modulating, which comprises inhibiting or promoting, the movement of said ECM generated by said mesothelial cells towards a site of injury of said organ of a subject suffering from or being at a risk of an injury of said organ by applying different compounds which will be defined in the following herein.

An “extracellular matrix (short: ECM)” according to the present invention refers to a collection of extracellular molecules secreted by cells. The ECM of the present invention may be composed of collagen fibrils, microfibrils, and elastic fibers, embedded in hyaluronan and proteoglycans. Preferably, said ECM comprises proteins, polysaccharides and/or proteoglycans. Those components may refer to ECM components according to the present invention. Such ECM components may be covalently coupled to a label which is used to contact the ECM, in particular the ECM components, in the screening methods as defined herein. Preferably, ECM may also comprise cells of fascia matrix, serosa and/or adventitia as described herein, such as mesothelial cells, macrophages, neutrophils, and/or fibroblasts, most preferably mesothelial cells.

Said ECM is mainly produced/generated by said mesothelial cells, which are the contributors of ECM production and ECM movement, thus also called mesothelial ECM movement In other words, said ECM comes from said mesothelial cells, which produce it as defined herein. Mesothelial derived ECM is made during development or maintenance of the internal organs and it is constantly generated during injury. In particular, producing ECM by said mesothelial cells means that said cells, when for example stimulated, secrete ECM molecules such as Type 1 Collagen, thus leading to increased expression of such molecules. Said molecules as defined herein are then formed to the ECM. In homeostasis, there is a reservoir of ECM beneath mesothelial cells. The expression level of ECM proteins in mesothelial cells is low and serves to maintain the reservoir. Upon injury, ECM proteins, which make up the reservoir, are transferred to the site of injury. In parallel, expression levels in mesothelial cells are strongly increased to (i) provide more proteins recruited to the site of injury and (ii) refill the reservoir. Thus, the replenishment of ECM, which has already moved away, with new ECM, which has newly been produced by said mesothelial cells, which is then moved again, mediates a constant flow of ECM, which is a central aspect in fibrosis.

Said mesothelial cells which produce the ECM also form the surface of said internal organ. Thus, said cells make up the surface of said organ of the subject defined herein. In other words, the surface of the internal organ consists of said cells as the outermost lining and the underlying ECM they produce as mentioned above. Thus, said surface of said internal organ is the outer serous membrane/layer as composed of said mesothelial cells and said extracellular matrix as defined herein.

The present inventors have now discovered that either all or a reactive fraction of said mesothelial cells are causative for the ECM production and for the movement of it, thus said mesothelial cells being the mediators of such mechanism, why a new clinical situation arises. Said term “mesothelial cells” as used herein refers to any cell that can be derived from the mesothelium known to a person skilled in the art. Thus, by knowing that said mesothelial cells produce the ECM, one can target said cells, thus modulating ECM movement towards said site of injury of said organ as defined elsewhere herein. Said modulation of the ECM movement as defined herein may thus also comprise that said compound is capable of targeting via direct or indirect mechanisms, preferably by direct mechanism as defined herein, mesothelial cells which form the outermost lining of said organ. Thus, said mesothelial cells are targeted and influenced by said compounds of the present invention, which will then modulate the movement of said ECM produced by said cells towards a site of injury of said organ of said subject as defined herein. In this context, the term “targeting said mesothelial cells” means that a compound of the present invention is capable of addressing mesothelial cells directly (e.g. via transduction of a compound as defined herein with i.e. a vector as defined herein which then targets said mesothelial cells) or that an applicable compound is capable of addressing said mesothelial cells indirectly via stimulation of immune cells, preferably of neutrophils, which then in turn secrete factors, which specifically target the mesothelial cells. Thus, by addressing immune cells with an applicable compound, immune cells become activated and in turn activate mesothelial cells (which refers to the indirect targeting of mesothelial cells, but may fall under the term of “targeting mesothelial cells”), whereby the movement of ECM produced by mesothelial cells may also be modulated. The term “specifically targeting” or “to target specifically” or “to target/targeting with sufficient/required specificity” means that said compound of the present invention as defined herein is only capable of addressing mesothelial cells directly or in other words, that said compound of the present invention particularly targets said mesothelial cells. With regard to the compound being a transcript as defined herein, the transduction of said transcript is more effective for mesothelial cells then for other cells, when said compound as defined herein is specifically targeting said mesothelial cells. In this context, when the term “targeting said mesothelial cells” or “specifically targeting said mesothelial cells” is used herein it is meant that all mesothelial cells or only a reactive fraction of said mesothelial cells are/is targeted by said compounds of the present invention, which is then sufficient for the modulation of the ECM movement. With regard to the compound being an antagonist or an agonist of a mesothelium specific receptor as defined herein, the term “specifically targeting” comprises the term “specifically binding” as defined elsewhere herein.

In this context, an “injury” may refer to 1.) a wound as defined herein or 2.) to any irritation, which does not refer to breaking the surface of the organ, but which may comprise any other disruption of the organ surface such as when using an acid as an irritation for example or 3.) to any other manifestations of irregularities of said internal organ surface. “A site of injury” may thus refer to an injured site—the location of an injury in said organ—which requires deposition of ECM. It is a site within said organ which signals a subject's body the requirement for ECM deposition. The signal is triggered by, e.g. an injury caused, e.g. by a wound. Usually, ECM deposition is required for patching a wound. Thus, a site of injury requiring ECM deposition is preferably a wound. A “wound” is a break in the continuity of any bodily tissue of a subject as defined herein due to, e.g. violence (such as a damaged surface of the organ's tissue), where violence is understood to encompass any action of external agency, including, for example, surgery. Said term includes open and closed wounds. The term “insult” may be used interchangeably for the term “injury” within the application.

The term “movement of ECM” or “ECM movement” “mesothelial ECM movement” may refer to the influx/invasion of said matrix towards said injured site of said organ as defined herein, thus referring to an out-to-inside movement of said matrix which is part of the outermost surface of said organ. When an injury occurred within said organ, the ECM, which is produced by said mesothelial cells, then moves from the outside (the surface of said organ) to the inside, where the injury is located within said organ.

In sum, the advantage of the present invention is that the compounds have a mesothelial (mesothelium)-specificity. Therefore, treatment or prevention options can be more specific. This could be enabled by (i) the injection route, which narrows down the potential targets and, in parallel, avoids adverse effects caused by non-mesothelial cells and/or (ii) increasing specificity of said compounds as defined elsewhere herein.

As described above, the term “modulating” or “modulation” comprises “inhibiting”, “inhibition”, or “promoting”, “promotion” of said movement of the ECM. Thus, both inhibition and promotion is of clinical value and therefore targetable. If a compound of the present invention inhibits the movement of ECM towards a site of injury of said organ as described elsewhere herein, this would then prevent excessive deposition of ECM at said site, thus blocking fibroproliferative disease. It is preferred that inhibition of ECM movement towards a site of injury which requires deposition of ECM prevents excessive ECM deposition at said site. Thus, excessive deposition of ECM is associated with fibroproliferative disease. In other words, matrix movement leads to excessive deposition of ECM, which then leads to/is associated with fibroproliferative disease. To treat or prevent a fibroproliferative disease would then mean applying a compound of the present invention which is able to inhibit ECM movement towards a site of injury which requires deposition of ECM. The term “inhibition” or “inhibiting” ECM movement may also comprise redirection/redirecting ECM, when said compound of the present invention may be applied, thereby stopping the matrix flow towards a site of injury as defined herein. Promotion of ECM movement would enable wound closure/repair in people with chronic wounds such as diabetes, aging, and in situations where local production of matrix is beneficial to close/repair wounds such as with abdominal and pelvic herniation, pneumothorax and so on. In other words, in some cases recruitment of ECM/accelerating ECM movement towards a site of injury of said organ as described elsewhere herein is also desirable and prevents insufficient deposition of ECM at said site, thus promoting impaired wound healing. It is preferred that promotion of ECM movement towards a site of injury which requires deposition of ECM prevents insufficient ECM deposition at said site. Thus, insufficient deposition of ECM is associated with a chronic wound. In other words, no/less (inhibited) matrix movement leads to insufficient deposition of ECM, which then leads to/is associated with a chronic wound. To treat or prevent a chronic wound would then mean applying a compound of the present invention which is able to promote ECM movement towards a site of injury which requires deposition of ECM.

Therefore, if modulation of ECM movement is inhibition, ECM movement is inhibited towards a site of injury which requires deposition of ECM, wherein said injury of said internal organ of said subject as defined herein may then be associated with a chronic wound. A “chronic wound” is a wound (preferably as defined herein) that does not heal in an orderly set of stages and in a predictable amount of time the way most wounds do; wounds that do not heal within about two to three months are usually considered chronic. For example, chronic wounds often remain in the inflammatory stage for too long and remain as opening in the skin and sometimes the deeper tissue. Chronic wounds may never heal or may take years to do so.

If modulation of ECM movement is promotion, ECM movement is promoted/accelerated towards a site of injury which requires deposition of ECM, wherein said injury of said internal organ of said subject as defined herein may then be associated with a fibroproliferative disease. A “fibrotic” disease or a “fibroproliferative” disease refers to a disease characterized by scar formation and/or the over production of extracellular matrix by connective tissue such as fibrosis and/or fibrous adhesion. Fibrotic disease may occur as a result of tissue damage of said organ (such as a wound) or of any irritation, which does not refer to breaking the surface of the organ, but which may comprise any other disruption of the organ surface or of any other manifestations of irregularities of said internal organ surface. It can occur in virtually every organ of the body of the subject as defined herein. Examples of fibrotic or fibroproliferative diseases include, but are not limited to, idiopathic pulmonary fibrosis, fibrotic interstitial lung disease, interstitial pneumonia, fibrotic variant of non-specific interstitial pneumonia, cystic fibrosis, lung fibrosis (also called pulmonary fibrosis), silicosis, asbestosis, asthma, chronic obstructive pulmonary lung disease (COPD), pulmonary arterial hypertension, liver fibrosis, liver cirrhosis, glomerulosclerosis, kidney fibrosis (also called renal fibrosis), uterus fibrosis (such as endometrial fibrosis), spleen fibrosis, pancreas fibrosis, brain fibrosis, cardiac fibrosis, bladder fibrosis, stomach fibrosis (also called intestinal fibrosis), diabetic nephropathy, heart disease, fibrotic valvular heart disease, systemic fibrosis, rheumatoid arthritis, keloids, excessive scarring for example resulting from surgery, e.g., surgery to fix hernia, chemotherapeutic drug-induced fibrosis, radiation induced fibrosis, macular degeneration, retinal and vitreal retinopathy, atherosclerosis, and restenosis, fibrous adhesion. Fibrotic disease or disorder, fibroproliferative disease or disorder are used interchangeably herein.

In a preferred embodiment, a fibroproliferative disease refers to any kind of fibrosis where matrix is abnormally built up/laid down in the internal organs, preferably being selected from the group consisting of lung fibrosis, liver fibrosis, kidney fibrosis, cardiac fibrosis, bladder fibrosis, brain fibrosis, spleen fibrosis, pancreas fibrosis, uterus fibrosis (in particular endometrial fibrosis) and stomach fibrosis. In another preferred embodiment, said term refers to excessive scarring and to keloids. Fibrosis, scarring and keloids all refer to intra-organ manifestations of a fibroproliferative disease. By said particular terms of “fibrosis” such for example “lung fibrosis”, said terms comprise all different forms of said particular fibrosis. The term “keloids” are abnormal scars with clinical features of early and unresolved wounds (e.g. itchiness, inflammation, and pain) that progressively grow beyond the injury site.

In another preferred embodiment, a fibroproliferative disease refers to fibrous adhesion which refers to an extra-organ manifestation of a fibroproliferative disease. The term “adhesion” or “adhesion formation” may refer to the binding of surfaces of internal organs from subjects as defined herein resulting from membrane attachments between opposing activated mesothelial cells.

The term “internal organ” of the present invention refers to any organ of the body of said subject as defined herein which is known to a skilled person in the art, preferably any one of a lung, a kidney, a heart, a liver, a stomach, a bladder, a peritoneum, a brain, a spleen, a pancreas, an uterus, a brain or an intestine, which comprises the large and the small intestine. Such term may also comprise the skin or any facial tissues. In a more preferred embodiment, said internal organ is any one of a lung, a kidney, a liver or a heart. In an even more preferred embodiment, said internal organ is a lung. Alternatively, in an even more preferred embodiment, said internal organ is a kidney. Alternatively, in an even more preferred embodiment, said internal organ is a liver. Alternatively, in an even more preferred embodiment, said internal organ is a heart. Such internal organ may further be defined as an internal organ of any of the cavities of said subject as defined herein which are known to the skilled person. The term “cavity” may comprise, but is not limited to, the “abdominal, lung and/or heart cavity”. The lung as internal organ belongs to the lung cavity. The heart as internal organ belongs to the heart cavity. The liver, the kidney, the bladder, the intestine, the stomach and/or the peritoneum as internal organs belong to the abdominal cavity. The spleen, the pancreas and/or the uterus belongs to the pelvic cavity. The brain belongs to the cranial cavity. Said internal organ as described herein may also be defined as “organ of the ventral cavity”. An organ of the ventral cavity may include any internal organ known to the skilled person which is part of the ventral cavity known to a person skilled in the art. Said term may comprise, but is not limited to organs such as lung, heart, kidney, liver, stomach, peritoneum, intestine, spleen, pancreas, uterus and bladder, preferably lung, kidney, heart and liver.

The term “a subject suffering from or being at a risk of an injury of said organ” refers to a vertebrate subject (also called just vertebrate). Such vertebrate includes any mammal, any reptile, any bird, any fish or any amphibian. Preferably, said subject is a mammal or a reptile. Even more preferably, said subject is a mammal. Most preferably, said subject is a human. Said subject as defined herein may already suffer from said injury of said organ as defined herein. In this instance, applying the compound of the present invention which will modulate the ECM movement may refer to some kind of treatment of said subject, preferably a treatment of a fibroproliferative disease as defined herein or of a chronic wound as already defined elsewhere herein. Alternatively, said subject as defined herein is at risk that an injury as defined herein of said organ might develop/occur which is then associated with either a fibroproliferative disease or a chronic wound. In this instance, applying the compound of the present invention which will modulate the ECM movement may then refer to some kind of prevention for said subject.

A compound for use in a method for the modulation of ECM movement towards a site of injury of said organ as defined herein can be any compound being mesothelial-specific, such as a small molecule or the like. Such compound may also comprise any gene editing tool known to a person skilled in the art such as CRISPR-Cas, when being mesothelial specific. Such a compound may also include cells or material from cells. When we refer to said compound as defined herein, said compound can also be bound to any medical device (such as a scaffold, a suture or any other device known to a skilled person). In the following different compounds of the present invention will be introduced.

### Transcription Construct Encoding a Gene Involved in the Modulation of Movement of ECM

In a first embodiment, said compound of the present invention is a transcription construct encoding a gene involved in the modulation of movement of ECM, which is produced by said mesothelial cells. The term “transcription construct” may also be called “genetic construct”. Such construct of the present invention comprises DNA or RNA. The term “DNA” as used herein comprises genomic and/or mitochondrial DNA. The term “RNA” as used herein comprises mRNA, miRNA, gRNA, and/or rRNA. A construct comprising DNA refers to a construct comprising a transcription template or it refers to a DNA construct. A construct comprising RNA refers to a construct comprising a transcription product or it refers to a RNA construct. This discrimination is important for the description of constructs containing promoters, which are only apparent in DNA-based transcription templates but not in the RNA-based transcripts.

If the transcription construct is a DNA construct, said construct further comprises a mesothelium specific control element and/or a mesothelium specific promoter element and/or a mesothelium specific enhancer element. Any control, promoter and/or enhancer element, also just called control, promoter, enhancer, which are mesothelial cells specific may be comprised herein. In this context, “mesothelium specific” means that said construct comprising one or more of the elements above will mediate functionality specifically in mesothelial cells as defined herein.

In detail, any promoter of all upregulated genes of the present invention associated with the activated status of said mesothelial cells may be used. In general, selection of a promoter depends on the application. If used for prevention, meaning that there is just the possibility, that mesothelial cells are activated, promoters associated with the activated status should be chosen. If used in a condition, when it is already known that all mesothelial cells are activated (e.g. after onset of lung fibrosis), also promoters constitutively expressed in mesothelial cell may be suitable. A preferred promoter element is the well defined MSLN-promoter. Alternatively, another preferred promoter is the constitutive mesothelial promoter M6A. Also alternatively, another preferred promoter is the activated mesothelial cell promoter HSF. Another preferred promoter which is mesothelium specific is any one of a CRIP1, LGALS1, MGP, SAA3 or a SEPP1 promoter. In one embodiment, said mesothelium specific promoter element is a CRIP1 promoter. In another embodiment, said mesothelium specific promoter element is a LGALS1 promoter. In another embodiment, said mesothelium specific promoter element is a MGP promoter. In another embodiment, said mesothelium specific promoter element is a SAA3 promoter. In another embodiment, said mesothelium specific promoter element is a SEPP1 promoter. In an even more preferred embodiment, said mesothelium specific promoter element is either a CRIP1 promoter or a MGP promoter, mostly preferred a CRIP1 promoter (see FIGS. 10 and 11).

The same applies mutatis mutandis to enhancer and/or control elements, thus e.g. applying an enhancer element of all upregulated genes used in the present invention. A preferred enhancer element of the present invention is the enhancer element of the human Cytomegalovirus (hCMV). Although the enhancement is unspecific, the increase in expression level is very high and the specificity will be mediated by the promoter. In a preferred embodiment, the present invention comprises the application of a control and an enhancer element, besides using a promoter element as defined herein. A control element may refer to a region of DNA adjacent to (or within) a gene that allows the regulation of gene expression by the binding of transcription factors. Some control elements are located close to the promoter (proximal elements) while others are more distant (distal elements). Regulatory proteins typically bind to distal control elements, whereas transcription factors usually bind to proximal elements. Both control elements may be used in the present invention and are thus comprised herein.

Said DNA construct and also said RNA construct (which does not comprise any promoter elements and the like) as defined herein may further comprise a RNA or protein target sequence. Preferably, said DNA construct and also said RNA construct as defined herein may further comprise a RNA target sequence. Such RNA target sequences may be recognized by cell-type specific mediators, which mediate the degradation of the RNA, therefore preventing RNA from translation. In this context, cell-type specific mediators may refer to RNAs, sugar-residues, proteins and the like. Even more preferably, said DNA construct and also said RNA construct as defined herein may further comprise a miRNA target sequence. A RNA-target sequence, in particular a miRNA target sequence, is also an element mediating cell specificity of said constructs. Said RNA target sequence may refer to some kind of control element of said DNA or RNA construct of the present invention. Normally, non target cells (non-mesothelial cells) would recognize such particular RNA target sequence and would then degrade the RNA, which would then result in no protein expression/immune response. Thus, said RNA target sequence, in particular said miRNA target sequence, is specific for non-mesothelial cells. Target cells (mesothelial cells) however do not degrade such RNA due to this specific RNA target sequence being comprised in the construct of the invention which is used as a compound for specifically targeting said mesothelial cells, therefore resulting in an expression of said RNA. In a preferred embodiment, the sequences used are targets for miRNAs, which are not expressed in mesothelial cells but cells, which should not be active with the defined compound of the present invention. Preferably, sequences are used which are recognized by miR-142-3p or miR-150-5p. miRNA target sequences may be characterized by one of several types of seed sequence matches unique to each miRNA as mentioned herein.

Said transcription construct as defined herein preferably encodes a particular gene which is involved in the modulation of movement of ECM, which is produced by mesothelial cells. Said gene is preferably selected from the group consisting of csta, tgfb, tgfbr2, ctsb, aebp1, col1a1, adamTs1, dcn, sparc, timp1, c1, c2, c3, c4, saa3, hsf1, and dtror a combination thereof (see also Table 1 below). Additionally or alternatively, said gene is preferably selected from the group consisting of mgp, crip1, plac8, lgals1, and ifi2712a or a combination thereof. Even more preferably, said gene is selected from the group consisting of csta, tgfb, dcn, sparc, tgfbr2, and ctsb or a combination thereof. Thus, said gene being involved in the modulation of the movement of ECM and encoded by the transcript as defined herein is csta. Said gene being involved in the modulation of the movement of ECM and encoded by the transcript as defined herein is tgfb. Said gene being involved in the modulation of the movement of ECM and encoded by the transcript as defined herein is dcn. Said gene being involved in the modulation of the movement of ECM and encoded by the transcript as defined herein is sparc. Said gene being involved in the modulation of the movement of ECM and encoded by the transcript as defined herein is tgfbr2. Said gene being involved in the modulation of the movement of ECM and encoded by the transcript as defined herein is ctsb. Said gene being involved in the modulation of the movement of ECM and encoded by the transcript as defined herein is mgp. Said gene being involved in the modulation of the movement of ECM and encoded by the transcript as defined herein is crip1. Said gene being involved in the modulation of the movement of ECM and encoded by the transcript as defined herein is plac8. Said gene being involved in the modulation of the movement of ECM and encoded by the transcript as defined herein is lgals1. Said gene being involved in the modulation of the movement of ECM and encoded by the transcript as defined herein is ifi2712a.

In other embodiments, the gene being involved in the modulation of the movement of ECM and encoded by the transcript as defined herein is aebp1. In another embodiment, the gene being involved in the modulation of the movement of ECM and encoded by the transcript as defined herein is col1a1. In another embodiment, the gene being involved in the modulation of the movement of ECM and encoded by the transcript as defined herein is adamTs1. In another embodiment, the gene being involved in the modulation of the movement of ECM and encoded by the transcript as defined herein is timp1. In another embodiment, the gene being involved in the modulation of the movement of ECM and encoded by the transcript as defined herein is c1. In another embodiment, the gene being involved in the modulation of the movement of ECM and encoded by the transcript as defined herein is c2. In another embodiment, the gene being involved in the modulation of the movement of ECM and encoded by the transcript as defined herein is c3. In another embodiment, the gene being involved in the modulation of the movement of ECM and encoded by the transcript as defined herein is c4. In another embodiment, the gene being involved in the modulation of the movement of ECM and encoded by the transcript as defined herein is saa3. In another embodiment, the gene being involved in the modulation of the movement of ECM and encoded by the transcript as defined herein is hsf1. In another embodiment, the gene being involved in the modulation of the movement of ECM and encoded by the transcript as defined herein is dtr.

According to the Examples of the present invention, the inventors demonstrated that an injury as defined herein may trigger mesothelial expression of TGFβ to activate Cathepsin B that enables matrix influx, causing or deteriorating a fibroproliferative disease as defined herein. Moreover, it was revealed that TGFβ and Cathepsin B are induced, at the top of the fibrosis cascade and that pharmacologic and genetic interventions against mesothelial TGFβ-mediated activation of mesothelial cells or expression of Cathepsin B by mesothelial cells inhibits matrix invasion (influx) and thus a fibroproliferative disease such as fibrosis.

By introducing said transcription construct as mentioned above which encodes for example the gene csta, Cystatin A is expressed, a direct inhibitor that binds and blocks Cathepsin B protease and that can be considered as one of the endogenous counter-acting proteins for inhibition of cathepsins. In line with that, suppression of mesothelial Cathepsin B by overexpressing Cystatin A stopped all matrix movements, thus inhibiting a fibroproliferative disease such as fibrosis, if the injury of said organ is associated with a fibroproliferative disease such as fibrosis (see FIG. 3). Vice versa by introducing said transcription construct encoding for example tgfb and/or ctsb, the TGFbeta and/or the Cathepsin B protease is/are expressed, leading to an increase in matrix influx, thus enabling wound closure/repair, if the injury is associated with a chronic wound.

By introducing said transcription construct as mentioned above which encodes for example the truncated gene tgfbr2, the dominant negative mutant of the TGFbeta receptor II is expressed. Said mutant then blocks the functionality of endogenous TGFbeta receptor complex. Inhibiting mesothelial TGFβ signaling alone completely blocked matrix buildup and matrix invasion (see FIG. 2).

TABLE 1
Selected genes and underlying path-way hereto.

| Gene name | Pathway |
|---|---|
| tgfbr2 | TGFbeta Signaling Pathway |
| tgfb | TGFbeta Signaling Pathway |
| aebp1 | Transcriptional Repressor |
| col1a1 | ECM Modification |
| adamts1 | Protease and Cell invasion |
| Cathepsin B (ctsb) | Protease |
| Cystatin A (csta) | Protease-Inhibitor |
| Decorin (dcn) | ECM-Interaction |
| sparc | ECM-Interaction |
| timp1 | Protease and Cell invasion |
| c1 | Immune system interaction |
| c2 | Immunesystem Interaction |
| c3 | Immunesystem Interaction |
| c4 | Immunesystem Interaction |
| saa3 | Immunesystem Interaction |
| hsf1 | Heat Shock factor Signaling |

By introducing said transcription construct as mentioned above which encodes for example the gene dtr, the Diphtheria Toxin receptor is expressed on mesothelial cells, to which the toxin diphtheria can then bind and kill the cells, if ECM inhibition may be required. Said particular construct may then also comprise particular mesothelium specific promoters as defined herein such as the MSLN promoter as a preferred one.

By introducing said transcription construct as mentioned above which encodes any one of the genes lgals1, dcn, sparc, or plac8, or a combination thereof, the corresponding protein(s) being expressed result(s) in inhibiting matrix movements, thus inhibiting a fibroproliferative disease such as fibrosis. By introducing said transcription construct as mentioned above which encodes the gene ifi2712a and/or mgp, the corresponding protein(s) being expressed result(s) in promoting matrix movements, thus enabling wound closure/repair, if the injury is associated with a chronic wound (see FIG. 9).

Thus, for inhibiting matrix movement, the gene being encoded by said transcription construct is any one of csta, tgfbr2, lgals1, dcn, sparc, or plac8 or a combination thereof. Thus, for promoting matrix movement, the gene being encoded by said transcription construct is any one of tgfb, ctsb, mgp or ifi2712a or a combination thereof.

### Antagonist or Agonist of a Mesothelium Specific Receptor

In a second embodiment, said compound of the present invention is an antagonist or an agonist of a mesothelium specific receptor. The term “antagonist” refers to receptor ligand that inhibits or reduces agonist-mediated biological responses rather than provoking a biological response itself upon binding to the receptor. Antagonists have affinity but essentially no efficacy for their receptors. The term also comprises antagonists binding to the active (orthosteric) or to allosteric sites of their receptors, and/or to other binding sites not normally involved in receptor function. The term “antagonist” in general comprises full and partial antagonists, reversible and irreversible antagonists. In accordance with the invention, the antagonist preferably specifically binds to a mesothelium specific receptor. The term “agonist” as used herein generally refers to a receptor ligand that activates the receptor upon binding to produce a biological response. In contrast to antagonists, agonists have both affinity and efficacy for their receptors. The term “agonist” in general comprises full and partial agonists, reversible and irreversible agonists.

The “antagonist” or “agonist” of the present invention may in general be any molecule, such as an antibody, a siRNA, a nucleic acid, an aptamer, a peptide, a protein, a lipid or a small organic molecule, that binds or specifically binds to a mesothelium specific receptor as specified herein, or a variant or a fragment thereof, and either blocks or reduces the biological responses mediated by a mesothelium specific receptor (i.e. acts as an antagonist) or activates the biological response mediated by a mesothelium specific receptor (i.e. acts as an agonist).

Antagonists and agonists can be easily found e.g. using screening assays known to the person skilled. The skilled person will readily acknowledge that ligands of proteins comprising e.g. a particular mesothelium specific receptor binding domain may be used as a template for preparing agents capable of binding to a mesothelium specific receptor and exhibiting an antagonistic or agonistic effect. E.g., in case of protein or peptide ligands, variants and fragments thereof can be easily prepared using routine methods of genetic engineering. The antagonists and agonists of the invention are envisaged to specifically bind to a mesothelium specific receptor described herein (i.e. preferably do not exhibit cross-reactivity towards targets other than a mesothelium specific receptor), as can easily be tested e.g. by evaluating antibody binding in mesothelium specific receptor knockdown host cells.

As set out herein, specific binding of the antagonists and agonists provided herein, e.g. antibodies, to the mesothelium-specific receptor is preferred. The terms “binding to” and “recognizing” in all grammatical forms are used interchangeably herein.

The term “specifically binds” generally indicates that a binding agent, in particular an antagonist or an agonist, such as an antibody, binds with higher affinity to its intended target (i.e. the mesothelium specific receptor described herein) than to its non-target molecule. Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹. Preferably, the term “specifically binds” thus indicates that an antagonist or an agonist, such as an antibody, exclusively binds to its intended target (i.e., the mesothelium-specific receptor).

In this context, with regard to the receptor of the invention, the term “mesothelial or mesothelium specific” means that said receptor is only expressed on mesothelial cells, but not on immune cells or any other cells. The receptor is expressed either at the surface of all mesothelial cells or at the surface of only activated mesothelial cells. Preferably, the receptor is expressed at the surface of activated mesothelial cells. Application of an antagonist for a receptor expressed at the surface of only activated mesothelial cells may lead to a deactivation of said cells, which is then considered a good treatment for any conditions, for which ECM movement plays a role. Thus, said antagonists of the present invention refer to a mesothelium-specific receptor ligand that inhibit or reduce mediated biological responses rather than provoking a biological response itself upon binding to said mesothelium-specific receptor.

In a preferred embodiment, the antagonist or agonist provided herein is an antibody. The antibodies provided herein preferably exhibit the desired biological activity, i.e. specifically binding to a mesothelium specific receptor described herein. As is well known in the art, an antibody is an immunoglobulin molecule capable of specific binding to a target (epitope) through at least one epitope recognition site, located in the variable region of the immunoglobulin molecule. The term “antibody” as used herein comprises monoclonal and polyclonal antibodies, as well as (naturally occurring or synthetic) fragments or variants thereof, including fusion proteins comprising an antibody portion with an antigen-binding fragment of the required specificity and any other modified configuration of the antibody that comprises an antigen-binding site or fragment (epitope recognition site) of the required specificity. Illustrative examples include dAb, nanobody, affibody, Fab, Fab′, F(ab′)₂, Fv, single chain Fvs (scFv), diabodies, and minibodies comprising a scFv joined to a CH3 domain. It will be understood that other antibody frameworks or scaffolds comprising “antigen-binding sites” can be employed in line with the present invention. The term “antibody” thus also comprises these scaffolds. The mentioned scaffolds include e.g. non-immunoglobulin based antibodies and scaffolds onto which CDRs of the antibodies can be grafted. Such scaffolds include for example anticalins, avimers, affilins etc.

siRNAs and nucleic acids may also be useful as mesothelium-specific receptor antagonists or agonists. The term “siRNA” is used interchangeably with “small interfering RNA” or “silencing RNA”. siRNAs are double-stranded “antisense” RNA molecules, typically including a sequence of at least 20 consecutive nucleotides having at least 95% sequence identity to the complement of the sequence of the target nucleic acid, but may as well be directed to regulatory sequences of said gene, including the promoter sequences and transcription termination and polyadenylation signals.

Other nucleic acids or gene editing techniques capable of reducing and/or inhibiting mesothelium-specific receptor expression may also include aptamers, Spiegelmers®, rRNAs, nc-RNAs (including anti-sense-RNAs, L-RNA Spiegelmer, silencer RNAs, micro-RNAs (miRNAs), short hairpin RNAs (shRNAs), small interfering RNAs (siRNAs), repeat-associated small interfering RNA (rasiRNA). Such non-coding nucleic acid molecules can for instance be employed to direct mesothelial-specific receptor mRNA degradation or disrupt mesothelial-specific receptor mRNA translation. Particularly, such gene editing techniques, which may be encoded by nucleic acid molecules, capable of reducing and/or inhibiting mesothelium-specific receptor expression may also refer to CRISPR-Cas9 gene editing tool. Such tool comprises a clustered, regularly interspaced, short palindromic repeats (CRISPR)-associated protein 9 (Cas9 protein) or a nucleic acid molecule encoding said Cas9; and a target sequence specific CRISPR RNA (crRNA) and a trans-activating crRNA (tracr RNA) or a nucleic acid molecule encoding said RNAs; or a chimaeric RNA sequence comprising a target sequence specific crRNA and tracrRNA or a nucleic acid molecule encoding said RNA.

Peptides, lipids and proteins can in general be employed as mesothelium-specific receptor antagonists or agonists, depending on whether they suppress (antagonists) or evoke (agonists) the biological responses mediated by said mesothelium-specific receptor signaling. The term “polypeptide” and “protein” are used interchangeably herein. It is envisaged that proteins, lipids and peptides bind specifically to a mesothelium-specific receptor as defined herein. As set out previously herein, the skilled person will readily be able to find peptide, lipids and protein antagonists or agonists capable of specifically binding to a mesothelium-specific receptor as defined herein. Said proteins, lipids and peptides can subsequently be tested for their antagonistic or agonistic activity using e.g. known screening assay.

Small organic molecules may also be used as agonists or antagonists of a mesothelium-specific receptor as defined herein and thus capable of acting as a mesothelium-specific receptor agonist or antagonist. It is envisaged that small organic molecules specifically bind to the mesothelium-specific receptor as defined herein. High-throughput screening assays for small organic molecules are readily available in the art and can be employed to find ligands of the particular mesothelium-specific receptors provided herein that may exhibit agonistic or antagonistic activity. Such small organic molecules may comprise, but may not be limited to sugars or other non-proteinaceous entities.

In a preferred embodiment, the mesothelium specific receptor is selected from the group consisting of MSLN1, GPM6A, PDPN, TGF-β receptor, LTB4 receptor BLT2, Podoplanin, and Procr. In an even more preferred embodiment, the mesothelium specific receptor is MSLN1, which is expressed on mesothelial cells, which are activated. By targeting said receptor with any MSLN1 specific antibody, activated mesothelial cells which express said receptor may then be deactivated, again modulating ECM movement for which said cells are the cause according to the present invention. The mesothelium-specific receptor to which an antagonist or agonist, preferably an antibody, specifically binds, can also be GPM6A. The mesothelium-specific receptor to which an antagonist or agonist, preferably an antibody, specifically binds, can also be PDPN. The mesothelium-specific receptor to which an antagonist or agonist, preferably an antibody, specifically binds, can also be TGF-β receptor. The mesothelium-specific receptor to which an antagonist or agonist, preferably an antibody, specifically binds, can also be LTB4 receptor BLT2. The mesothelium-specific receptor to which an antagonist or agonist, preferably an antibody, specifically binds, can also be Podoplanin. The mesothelium-specific receptor to which an antagonist or agonist, preferably an antibody, specifically binds, can also be Procr. In a most preferred embodiment, the mesothelium specific receptor is GPM6A (see FIG. 11).

In a further embodiment of the present invention, the application of said transcript as defined herein can also be combined with applying an antagonist or agonist of said mesothelium specific receptor (preferably an antagonist, such as an antibody of the MSLN1 receptor) as also described herein for modulating ECM movement produced by mesothelial cells.

### Administration of Said Compounds

Said compounds defined herein may be administered to the cavity, where the internal organ is located, via injection or infusion. Where the composition is to be administered by infusion, it can be dispensed with an infusion bottle containing sterile pharmaceutical grade water or saline. Infusion can also comprise delivering it in alburex or associated diluents if the compound requires a stabilisation protein. Where the composition is administered by injection, an ampoule of sterile water for injection or saline can be provided so that the ingredients may be mixed prior to administration. Said compounds as defined herein may be administered as a liquid form, in particular as an aqueous form, or as a solid form such as a gel.

In a preferred embodiment, the administration of said compound is performed intraperitoneally, intravenously, intrapleurally, intrathecally, via pericardiocentesis or via the lymphatic system. Thus, if the compound is to be administered to the lung (to the lung cavity), or to the liver, kidney, intestine, bladder, stomach, peritoneum (or to any other internal organ located in the abdominal cavity) or to the spleen, uterus, pancreas (or to any other internal organ located in the pelvic cavity), said compound of the present invention may either be administered intraperitoneally or intrapleurally or also intravenously or via the lymphatic system. If the compound is to be administered to the heart (to the heart cavity), said compound of the present invention may be administered via pericardocentesis (particularly via the central line). If the compound is for example also to be administered to the brain (to the cranial cavity), said compound of the present invention may be administered intrathecally (in particular via a shunt). Therefore, the administration route of the compound of the present invention depends on the organ as defined herein which will be targeted. In an even more preferred embodiment, the administration of said compound is performed intraperitoneally, intrapleurally, or via pericardiocentesis.

In a preferred embodiment of the present invention, the compound as defined herein is administered via a viral vector. In another preferred embodiment, said compound as defined herein is administered via a liposome. In another preferred embodiment, said compound as defined herein is administered via a transfection reagent. In another preferred embodiment, said compound as defined herein is administered via an extracellular vesicle. In another preferred embodiment, said compound as defined herein is administered directly. If the compound per se is administered as defined herein, this refers to a direct administration of said compound without any application of a viral vector as defined herein, a liposome, a transfection reagent, or an extracellular vesicle as defined herein. In a most preferred embodiment of the present invention, the compound as defined herein is administered via a viral vector.

A viral vector as used herein may comprise an adeno-associated virus (short: AAV) vector and an adeno-virus (short: AV) vector. Preferably, the viral vector used herein refers to an AV vector and/or to a capsid-modified AAV vector, even more preferably wherein the capsid-modified AAV vector refers to an AAV serotype 8 (AAV8) vector comprising a particular RGD peptide as will be defined herein. Both vectors enable transduction of said compound into mesothelial cells with equal efficacy. Serotype 8 has been identified as the most promising candidate serotype capable of transducing mesothelial cells. However, other serotypes might be also suitable for targeting mesothelial cells after incorporation of the peptide sequence comprising a RGD motif (e.g. the RGD-containing peptide). If the compound is administered as defined herein via an AV vector, said vector may not need to be genetically engineered. In other words, the capsid of said AV vector may not need to be genetically engineered as the RGD motif is encoded in one of the capsid proteins. If the compound is administered as defined herein via an AAV vector, said vector may comprise a peptide sequence which comprises a RGD motif. In other words, only after incorporation of the peptide sequence comprising a RGD motif into any one of the capsid proteins of the AAV vector via genetic engineering, said AAV vector, preferably an AAV serotype 8 vector, mediates efficient transduction of mesothelial cells. Thus, RGD-peptide incorporation for AAV vectors enhances efficiency of targeting mesothelial cells.

According to the present invention, if the compound is a DNA construct as defined herein, said compound is to be administered preferably via a viral vector, such as via an AV vector or via an AAV vector (preferably an AAV serotype 8 (AAV8) vector). Even more preferably, according to the present invention, if the compound is a DNA construct as defined herein, said compound is to be administered via an AAV vector (preferably an AAV serotype 8 (AAV8) vector) comprising a peptide sequence comprising a RGD motif.

Said particular peptide sequence enables a specific binding to integrin receptors. The integrins recognizing the RGD-motif are expressed in several cell types, thus also in mesothelial cells. Therefore, said peptide comprising said RGD motif which is comprised by said viral vectors as defined herein is not mesothelial specific. However, since it was demonstrated by the inventors that said cells are the mediators of ECM movement, the combination of the RGD-mediated targeting due to the fact that integrin receptors are also expressed on mesothelial cells with the local application as defined herein, makes a specific targeting of said cells possible when modulating ECM movement. In particular, said specific peptide used in the present invention may be incorporated into the capsid protein VP1 of said AAV vector. Said peptide as defined herein may also be incorporated into the capsid protein VP2 of said AAV vector. Said peptide as defined herein may also be incorporated into the capsid protein VP3 of said AAV vector. Said peptide as defined herein may also be incorporated into any one of the capsid protein VP1, VP2 and/or VP3 of said AAV vector. The inserted peptide comprising the RGD-motif may also be present in all VPs (VP1, VP2 and VP3) of said AAV vector. VP2 and VP3 are proteins identical to VP1, but missing small N-terminal parts. In the context of the present invention, when the term an AAV vector as defined herein comprises a peptide comprising a RGD motif, it means that said peptide comprising said RGD-motif may be incorporated into any one of VP1, VP2 or VP3 (or into all) capsid proteins of said AAV vector as defined herein. In particular, when the AAV serotype 8 (AAV8) vector) is applied herein, which comprises the peptide comprising a RGD motif (also called AAV8RGD), the peptide may be incorporated in between amino acids 484 and 485 within the VP1 protein. Therefore, the modification is present in all VPs (VP1, VP2 and VP3). However, also other incorporation sites (as used for other capsid-modified AAVs) might be suitable to mediate efficient transduction.

In a preferred embodiment said peptide, which comprises the RGD-motif, refers to

SEQ ID NO: 1
(TGCDCRGDCFCG).

The RGD motif is marked by the underlining with high accessability of said RGD-motif for cellular integrins. The flanking sequences are responsible for the optimal presentation on the AAV capsid surface of said vector of the present invention.

According to the present invention, any peptide may be used which comprises the RGD-motif and where the flanking sequences are responsible for the optimal presentation on the AV or the AAV capsid surface of said vector. Preferably, said peptide may comprise an amino acid sequence having at least about 70% identity with an amino acid sequence of SEQ ID NO.: 1. In particular, the peptide comprising a RGD-motif as described herein may comprise an amino acid sequence having at least about 70% identity, at least about 75%, at least about 80%, at least about 85%, at least about 90%, at least about 95%, including at least about 96%, 97%, 98%, 99% or even 100% sequence identity with the amino acid sequence of with an amino acid sequence of SEQ ID NO.: 1.

If the compound is a RNA construct as defined herein, said compound is to be administered preferably via said liposome. According to the present invention, if the compound is a transcription construct, preferably a RNA construct as defined herein, said compound may also be administered directly to said subject in need thereof via injection or infusion as defined herein, meaning as “naked mRNA” without using any viral vector, liposome, transfection reagent or extracellular vesicle as defined elsewhere herein.

According to the present invention, if the compound is an antagonist or agonist of a mesothelium-specific receptor as defined herein, preferably an antibody, even more preferably an antibody of the MSLN1 receptor, said compound may also be administered directly to said subject in need thereof via injection or infusion as defined herein without using any viral vector, liposome, transfection reagent, or extracellular vesicle as defined elsewhere herein.

Said compound may also be administered as defined herein via a transfection reagent. In this context, said compound may be a transcription construct as defined herein. Said transfection reagent may be used to introduce naked or purified nucleic acids such as naked DNA or RNA as a compound of the present invention into eukaryotic cells. Said reagent refers to, but is not limited to, Lipofectamine.

Presently, three main subgroups of extracellular vesicles as used herein have been defined in the scientific literature: a) apoptotic bodies, b) cellular microparticles (also termed “microvesicles” or “ectosomes”), and c) exosomes (cf. Yanez-M6 et al., Journal of Extracellular Vesicles 2015, 4: 27066). Apoptotic bodies usually have a size ranging from about 1 to 5 μm diameter and are released when plasma membrane blebbing occurs during apoptosis, while the second group comprises vesicles of different sizes that pinch directly off the plasma membrane and have a size of about 100 to 1000 nm diameter. Exosomes have a size of about 30 to 100 nm diameter and are usually intraluminal vesicles (ILVs) contained in multi-vesicular bodies (MVBs), which are released to the extracellular environment upon fusion of MVBs with the plasma membrane (Colombo et al., Ann Rev Cell Dev Biol. 2014; 30:255-89). In the present invention preferably exosomes may be applied which the compound as defined herein is administered with.

### In Vivo Screening Method

In another embodiment, the present invention comprises the compounds of the present invention used in an in vivo screening method for identifying a modulator of movement of extracellular matrix (ECM) produced by mesothelial cells towards a site of injury of an internal organ of a subject. Said in vivo screening method is based on the inclusion of matrix labeling and fate mapping and establishes a new in vivo model in living organism. Each definition with regard to the compound or any other term being used with regard to the screening method and which previously has defined herein may also be applicable here.

The method for identifying a modulator/modulators of ECM movement towards a site of injury of an internal organ as defined herein includes labelling of the ECM. Hence, by labelling ECM, the ECM is visualized for being observed. Observation of ECM movement allows the identification of modulators of ECM movement, since a modulator may either decrease/inhibit or accelerate/promote ECM movement. As explained, visualization of the movement of labelled ECM allows the identification of a modulator being an inhibitor of ECM movement on the basis of decreasing/inhibiting ECM movement, while a modulator being a promoter of ECM movement can be identified on the basis of accelerating/promoting ECM movement. Without being bound by theory, it is assumed that decreasing/inhibiting ECM movement will result in a decreased deposition of ECM at a site requiring ECM deposition, such as a wound, while accelerating/promoting ECM movement will result in an accelerated deposition of ECM at a site requiring ECM deposition, such as a wound.

Decreasing ECM movement when used herein is equivalent to inhibition of ECM movement. Inhibition of ECM movement towards a site requiring ECM deposition preferably prevents excessive deposition of ECM at said site.

Accelerating ECM movement when used herein is equivalent to promotion of ECM movement. Promotion of ECM movement towards a site requiring ECM deposition preferably prevents insufficient deposition of ECM at said site.

“Identifying modulators of ECM movement” or “identification of modulators of ECM movement” includes screening such modulators and, once identified or screened, isolating, i.e. providing such modulators.

The term “in vivo” refers to “ex vitro” and may be used interchangeably herein. Thus, the screening method is performed in a living subject. In this context, the term “subject” refers to any living organism such as a vertebrate as defined elsewhere herein, preferably a mammalian subject. A mammalian subject may refer to any mammal known to a person skilled in the art. Preferably, said mammalian subject is a human, a non-human primate, a mouse or a rat.

### Step (a)

The definition of said internal organ as used herein may also be applicable here. The internal organ which is used for contacting the ECM of said organ with a label preferably refers to a lung, a kidney, a heart, a liver, a stomach, a bladder, a peritoneum, a spleen, a brain, a pancreas, an uterus, or an intestine as defined elsewhere herein, even more preferably said organ is any one of a lung, a kidney, a liver or a heart.

When in step a) of the method of the present invention the term “contact” or “contacting” is used, it means that said ECM of said organ, preferably of said organ surface which comprises the mesothelial cells as well as the ECM as defined elsewhere herein is brought into contact with said label, which covalently couples to said ECM components of said ECM. In a preferred embodiment, the term “contact” or “contacting” refers to “selectively contact” or “contacting”. In this context, “selectively contacting” means that not the whole ECM of the organ is contacted with said label as defined elsewhere herein, but one or more portion of said ECM of said organ surface. In other words, when the term “selectively contacting” is used herein, a confined very specific spot of the ECM of said organ is contacted with said label as defined elsewhere herein, thus performing a locally ECM labelling on the organ of the present invention. Preferably, proteins comprised by said ECM are labelled. However, it is also envisioned that other components of ECM may be labelled, such as carbohydrates. In this context, contacting may comprise administering a label as defined herein to said subject used in the in vivo screening method in order for the label to be brought in contact with the ECM of said organ surface it needs to label. Here, the term administering may refer to an administration via injection or infusion or orally, preferably injection. Administration may be performed systemically or locally. The term systemically may refer to enterally such as orally; parenterally via injection or infusion such as intravenously, intrathecally, intraperitoneally or intrapleurally or via the lymphatic system; or rectally. In a preferred embodiment, contacting comprises the administration of said label to said subject intravenously, intrathecally, intraperitoneally or intrapleurally, via pericardiocentesis or via the lymphatic system (which refers to locally), depending on the organ which needs to be targeted as defined elsewhere herein. Meaning if it is a lung fibrosis model, the lung surface may be labeled by intrapleural injection with the label, preferably with NHS esters. If it is a liver fibrosis model, the liver surface may be labeled by intraperitoneal injection with the label, preferably with NHS esters and so on.

A “label” is a molecule or material that can produce a detectable (such as visually, electronically or otherwise) signal that indicates the presence and/or concentration of the label in a sample from an organ tissue. Thereby, e.g., the presence, location and/or concentration of a labelled molecule in a sample can be detected by detecting the signal produced by the (detectable) label. A label can be detected directly or indirectly. It will be appreciated that the label may be attached to or incorporated into a molecule, for example, a protein, polypeptide, or other entity, at any position. It will be appreciated that, in certain embodiments, a label may react with a suitable substrate (e.g., a luciferin) to generate a detectable signal. In particular, the detectable label can be a fluorophore, an enzyme (peroxidase, luciferase), a radiolabel, a fluorescent protein. Other detectable labels include chemiluminescent labels, electrochemiluminescent labels, bioluminescent labels, polymers, polymer particles, metal particles, haptens, and dyes.

A “fluorophore” (or fluorochrome) is a fluorescent chemical compound that can re-emit light upon light excitation. Examples of fluorophores include 5-(and 6)-carboxyfluorescein, 5- or 6-carboxyfluorescein, 6-(fluorescein)-5-(and 6)-carboxamido hexanoic acid, fluorescein isothiocyanate, rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, inorganic fluorescent labels such as particles based on semiconductor material like coated CdSe nanocrystallites.

Examples for fluorescent proteins include e.g., Sirius, Azurite, EBFP, EBFP2, TagBFP, mTurquoise, ECFP, Cerulean, CyPet, TagCFP, mTFPI, mUkGI, mAGI, AcGFPI, TagGFP2, EGFP, GFP, mWasabi, EmGFP, YFP, TagYPF, Ypet, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, mKalama2, T-Sapphire, mAmetrine, mKeima, UnaG, dsRed, eqFP611, Dronpa, KFP, EosFP, Dendra, and IrisFP.

Examples of enzymes used as enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO).

Examples of radioactive labels (radiolabel) include radioactive isotopes of hydrogen, iodide, cobalt, selenium, tritium, carbon, sulfur and phosphorous. ²H, ³H, ¹³C, ¹⁴C, ¹⁵N, ¹⁸F, ³¹P, ³²P, ³⁵S ⁶⁷Ga, ⁷⁶Br, ^{99m}Tc (Tc-99m), ^{m}In, ¹²³I, ¹²⁵I, ¹³¹I, ¹⁵³Gd, ¹⁶⁹Yb, and ¹⁸⁶Re.

According to the present invention, said label comprises preferably a dye and/or a tag by which ECM may be labeled. When said label is a dye, a fluorescent dye is preferred. A fluorescent dye may refer to a reagent coupled to a fluorophore. In particular, said reagent refers to N-Hydroxysuccinimide ester or Succinimidyl esters (NHS) or sulfodichlorophenol (SDP)-ester. In a preferred embodiment, said reagent refers to N-Hydroxysuccinimide ester/Succinimidyl ester (NHS-ester). When used in the present invention NHS ester means N-hydroxysuccinimide ester or Succinimidyl esters. NHS or SDP-esters react with extracellular amines, like N-termini of proteins and lysines labelling ECM-components. NHS/SDP esters conjugated with fluorophores as defined herein (such e. g. as Alexa 488, Alexa 568, Alexa 647, Fluorescein, Fluorescein isothiocyanate (FITC), Pacific Blue), may be used to visualize ECM. A fluorescent dye, preferably NHS-ester coupled to a fluorophore as defined herein, or a radiolabel as defined herein is preferred herein as a label used in the screening method.

As apparent from the above a NHS ester is sufficient to label extracellular amines. An essential step in untangling the phenomenon of ECM movement is the possibility to crosslink of moved material in the wound areas. Primary amines of proteins and peptides of distinct protein classes are covalently linked. In one preferred embodiment the NHS ester of the present invention may be used to label primary amines. Amines are compounds and functional groups that contain a basic nitrogen atom with an ion pair. They can be classified according to the nature and number of substituents on nitrogen. In nature there are primary, secondary and tertiary amines. Primary amines (also called primary amine groups) arise when one of three hydrogen atoms in ammonia is replaced by an alkyl or aromatic group. Important primary alkyl amines include, methylamine, most amino acids, while primary aromatic amines include aniline. According to the method of the present invention, primary amine groups of certain amino acids of said ECM components as defined elsewhere herein are labelled by said label as described above. In a preferred embodiment, primary amine groups of lysine of said ECM components as defined elsewhere herein are labelled.

An amine staining by Succinimidyl (NHS)-ester labelling has its effect in labelling all amine-containing ECM components and is not selective like antibodies which label one specific targets. The staining was developed for dead tissue and needs an alkaline pH, thus was assumed to damage living tissue. Thus, currently there are no reports on NHS/SDP-ester usage on living tissue, so no methods exists to visualize all amine-containing ECM molecules on organs.

The NHS ester labeling might be used in a diagnostic approach. For diagnostic studies, NHS ester labelling can be used for detecting a fibroproliferative disease such as fibrosis and the disease progression of said disease by exploring the relative abundance of NHS esters. A diagnostic approach might also be to monitor wound healing or wound progression. In this scenario it might be advantageous to combine NHS ester with a further reporter molecule as described above. In one preferred embodiment the NHS ester stain might be combined with any kind of reporter or fluorescent dye.

Preferably, such fluorescent dye include, but is not limited to, Alexa Fluor 488 NHS-ester, NHS-Fluorescein (5/6-carboxyfluorescein succinimidyl ester), Alexa Fluor 568 NHS-ester, Pacific Blue Succinimidly Ester, Alexa Fluor 647 NHS-ester (N-hydroxysuccinimide ester or Succinimidly Ester), Alexa Fluor 488 5-SDP-ester or NHS-Rhodamine (5/6-carboxy-tetramethyl-rhodamine succinimidyl ester). Each of the above mentioned fluorescent dyes are able to label the ECM components of the ECM matrix from each organ tissue described elsewhere herein.

Said NHS ester might be administered (preferably via injection) systemically or locally, preferably intraperitoneally or intrapleurally or even intravenously, intrathecally, or via the lymphatic system (which refers to systemically) or via pericardiocentesis (which refers to locally), depending on the organ which needs to be targeted as defined elsewhere herein. In another scenario, the NHS ester might be coupled to a compound to target the ECM systemically. In case NHS ester is coupled or linked to a compound, any kind of compound might be suitable. However, preferred are therapeutic compounds. The compound coupled to NHS ester might also be a modulator of the extracellular matrix (ECM) movement as described herein, which refers to the compound of interest.

Also comprised herein is that the label used in the method of the present invention comprises a tag. A “tag” can be an affinity tag (also called purification tag), such as a Biotin tag, histidine tag, Flag-tag, streptavidin tag, strep II tag, an intein, a maltose-binding protein, an IgA or IgG Fc portion, protein A or protein G. Preferably, said tag which is used in the method of the present invention and also conjugates with NHS/SDP esters is a Biotin tag. Such tags as defined elsewhere herein can thus also be used to analyze ECM components via protein biochemistry, like western blotting or mass spectrometry. According to the present invention, NHS-ester coupled to tag, preferably a Biotin tag, is also preferred herein as a label used in the screening method.

The method of the present invention may also be extended by further comprising step (a′) namely contacting said organ surface as defined herein with a label visualizing cells, preferably mesothelial cells, comprised in the ECM. In this context, said label refers to a lipophilic membrane fluorescent dye that spread through lateral diffusion capturing the entire cells. The additional labelling step may be performed before or after contacting the ECM of said organ with the first label as described elsewhere herein. Such membrane staining may be helpful to better identify/trace the ECM movement towards a site of injury in said organ which requires deposition of ECM.

### Step (b)

After having contacted the ECM of said organ of the subject with said label as defined herein, an injury according to the present invention is then introduced to said organ. By the term “introduce” or “introducing” it means that any violation is performed on the (surface of said) organ that an injury as defined herein originates/arises. In this particular context, the term “violation” may refer to breaking the surface of said organ that a wound arises or performing any other irritation/disruption of said organ or performing any other manifestations of irregularities on the surface of said organ.

In a preferred embodiment to introduce an injury to a particular organ, a medication may be administered to said subject. A medication includes, but is not limited to bleomycin, carbon tetrachloride (CCl4), LPS or Zymosan. Thus, step b) preferably comprises introducing to said organ an injury by administering to said subject as defined herein bleomycin, if lung fibrosis should be introduced. Also, step b) preferably comprises introducing to said organ an injury by administering to said subject as defined herein carbon tetrachloride (CCl4), if liver fibrosis should be introduced. Also, step b) preferably comprises introducing to said organ an injury by administering to said subject as defined herein LPS or Zymosan, if peritoneal fibrosis should be introduced. In this context, “administering” or “administration” may refer to injection or infusion or orally as defined herein, preferably injection. Again, said medication introducing an injury to said organ is preferably administered intraperitoneally, intrathecally, intravenously, intrapleurally or via pericardiocentesis or via the lymphatic system, depending on the organ which is targeted as defined elsewhere herein.

When bleomycin is applied/administered as a medication to introduce an injury in said organ, such as lung, it is administered via injection in the trachea. When carbon tetrachloride (CCl4) is applied/administered as a medication to introduce an injury in said organ, such as liver, it is administered via intra-peritoneal injection. When LPS or Zymosan is applied/administered as a medication to introduce an injury in said organ, such as peritoneum, it is administered via peritoneal injection.

### Step (c)

When in step c) of the method of the present invention the term “contact” or “contacting” is used, it refers that said compound of interest that is tested whether it modulates ECM movement towards a site of injury of said organ is again brought into contact with the mesothelial cells which form the surface of said organ defined herein and which is targeted. Contacting may again comprise administering said compound to said subject used in the in vivo screening method as defined elsewhere herein. In this context, “administering” or “administration” may refer to injection or infusion or orally as defined herein, preferably injection. Even more preferably, administration of said compound of interest is performed microdermally, intraperitoneally, intravenously, intrathecally, intrapleurally, via pericardiocentesis or via the lymphatic system or via cavity wash, depending on the organ which is targeted as defined elsewhere herein. After having administered said compound of interest to said subject as defined herein, said compound then contacts the mesothelial cells which form the outermost surface of said organ which is targeted due to the particular mesothelium specificity as discussed elsewhere herein.

The term “compound of interest” refers to a compound which is tested in the method of the present invention in order to identify whether said compound is a modulator of said ECM movement. Such modulator can be an inhibitor, thus inhibiting said ECM movement towards a site of injury of said organ, once the inhibitor is contacted with said mesothelial cells forming the outermost surface of said organ. However, such modulator may also refer to a promoter/an inducer, thus promoting/inducing said ECM movement towards a site of injury of said organ, once the promoter is contacted with said mesothelial cells. Preferably, the compound of interest may be an inhibitor. Even more preferably, said compound of interest refers to a transcription construct as defined herein or an antagonist of a mesothelium specific receptor as also defined herein.

In another embodiment of the invention, step b) and step c) can also be switched. This means that also step c) the contacting of said mesothelial cells with the compound of interest by administering said compound of interest to said subject as defined herein is applied before step b) the introduction of an injury as defined also herein. If this is the case that administration of the compound of interest is applied before the injury instillation, this may refer to a preventive treatment, whereas if step b) is before step c) as mentioned above, this may refer to a classical treatment.

### Step (d)

When the term “to determine” or “determining” also called “to detect” or “detecting” in step d) of the method of the present invention is used herein, it may be done or achieved by using any detection method such as contrast CT, MRI or X-rays. The term “detection method” when used herein refers to an imaging method such as a visual inspection, or to a protein biochemistry method thereby collecting imaging data. The term “visual inspection” refers to the visualization whether said compound of interest indeed modulates ECM movement as defined elsewhere herein by using a microscope, preferably by using a fluorescence stereomicroscope, or by using any one of contrast CT, MRI, mass spectrometry or X-rays or even an automated visual inspection analysis i.e. algorithmical analysis from microscope generated images. A protein biochemistry method can also include Raman spectroscopy known to a person skilled in the art. This detection by any imaging method as defined herein or even by any protein biochemistry methods known to a person skilled in the art is performed in comparison to a control subject as defined herein, which has its ECM of said organ surface also labelled as defined elsewhere herein, but which does not have said mesothelial cells of said organ surface contacted with said compound of interest. Thus, step a) and step b) of the in vivo method have also been performed for said control subject, only step c) the contacting of said cells with a compound of interest has not been performed for said control subject.

The control subject is the same organism as the subject used in the in vivo screening method—meaning if the subject of the in vivo screening method is a human, the control subject is also a human or if the subject of the in vivo screening method is a mouse, the control subject is also a mouse. In other words, the subject and the control subject are the same.

The signal in the imaging data received during the detection/determination step can be considered as the reflected signal received from the label used to contact said ECM of said organ. The method may further comprise comparing the imaging data received from the imaging method as defined herein to reference imaging data from said control subject. When comparing the collected imaging data from the subject to reference imaging data from the control subject, the method comprises comparing the movement of said labelled ECM towards said site of injury of said organ which is determined by the imaging method as defined herein after having contacted the mesothelial cells of said organ of said subject with said compound of interest to the movement of said labelled ECM towards said site of injury of said organ of said control subject, where the mesothelial cells of said organ surface have not been contacted with said compound of interest. This is comprised by the term “in comparison to” or “comparing to”. If by applying the imaging method and by comparing the imaging data as defined above, it is determined that there is a decrease/inhibition of said ECM movement towards said site of injury of said organ of the subject, the compound of interest may be considered as being an inhibitor, thus having inhibition of ECM movement as modulation. If by applying the imaging method and by comparing the imaging data as defined above, it is determined that there is a promotion of said ECM movement towards said site of injury of said organ of the subject, the compound of interest may be considered as being a promoter, thus having promotion of ECM movement as modulation.

The present invention also comprises an in vivo screening method for identifying a modulator of the movement of extracellular matrix (ECM) produced by mesothelial cells towards a site of injury of an internal organ of a subject, the method comprising
a) contacting ECM of an internal organ of a subject with a label;
b) introducing to said organ an injury;
c) contacting mesothelial cells, which form the surface of said organ with a compound of interest;
d) determining whether said compound of interest modulates movement of ECM towards a site of injury of said organ using a detection method in comparison to a control subject having ECM of said organ labelled, but not having mesothelial cells of said organ contacted with said compound of interest,
wherein modulation of the movement of ECM towards said site of injury of said organ is indicative for said compound of interest to be a modulator of said ECM movement and wherein step b) and step c) can be switched. Such definitions made above may also be applicable here.

### In Vitro Screening Method

In another embodiment, the present invention comprises an in vitro screening method for identifying a modulator of the movement of extracellular matrix (ECM) towards an external stimulus in a mesothelial single cell suspension. Said in vitro screening method can be performed manually or automatically. Either all steps of said screening method may be performed then manually or automatically or only some steps may be performed manually or automatically. Said method provides a basic knowledge for implementation into ex vivo organ/organoid cultures and in understanding whole system in vivo animal responses. Said method can be used in screening of compounds for induction of matrix movement, establishing mode of action at a single cell level, determining toxicity, pharmodynamics and pharmacokinetics at a single cell level. Each definition with regard to the compound or any other term being used with regard to the screening method and which previously has defined herein may also be applicable here, if needed.

### Step a)

The “single cell suspension (derived) from the mesothelium” as used herein refers to a mixed population of cells derived from the mesothelium/derived from the mesothelial layer, thus a heterogeneous population of any cells derived from mesothelium/mesothelial layer (immune cells, epithelial cells, stromal cells) or it refers to a pre-selected purified population of cells derived from the mesothelium/derived from the mesothelial layer, thus a pre-selected purified mesothelial subpopulation (e.g. just stromal cells, just immune cells or just epithelial cells). The term “mesothelial single cell suspension” may also be used interchangeably herein.

When the term “contacting said suspension with an already labeled ECM” is used herein, it means that said single cell suspension from the mesothelium is placed on or with already labeled ECM, which has already been produced in vitro. Such matrix may then refer to an exogeneous matrix, which has been purified before and was not produced naturally by the cells within said suspension. Thus, said exogeneous matrix does not belong to the mesothelial cells within the suspension used herein. Said labeling of the exogeneous ECM may be performed as discussed for the in vivo screening method. A labeled ECM in this context may thus refer to an ECM which has been contacted with a label as defined elsewhere herein. Preferably, said ECM has been labeled with NHS-ester coupled to a Biotin tag, labeled with a fluorescent dye, such as NHS-ester coupled to a fluorophore or labeled with a radiolabel as defined herein. As an alternative, said suspension can also be placed under suitable conditions, which comprise placing said suspension in a suitable cell culture known to a person skilled in the art, which then allows for said mesothelial cells to produce their own ECM naturally. Said ECM which was then produced by said mesothelial cells comprised by the single cell suspension placed under suitable conditions is then labeled/contacted with a label as defined elsewhere herein. Such ECM may refer to an endogenous matrix, since said matrix belongs to said mesothelial cells within the suspension. These two alternatives can be performed in a 2D and/or 3D system (e.g. using a (plastic) plate where the exogenous matrix can be placed on or where the cells could be placed on which then naturally produce their own ECM; or using a gel where the cells of the suspension grow within and then produce their own ECM or where the gel is formed by said exogenous matrix where the cell suspension is then placed onto).

### Step b)

In this context, the term “external stimulus” refers to, but is not limited to an injury as defined herein (also defined as an insult to the cells herein), a chemical stimulation, or a chemotactic gradient. These exposition steps, which are known to a person skilled in the art, may also comprise exposing said single cells within said suspension to several external stimuli, thus to any combination of the stimuli as defined herein. Here, the definition of an injury with regard to cells and not to an organ may be applicable here. It may also be comprised herein that the external stimulus may be the compound of interest. This would be the case if said compound of interest for example refers to a drug or a chemotractant or the like. In that case step b) refers to step c).

### Step c)

The contacting step in step c) with the compound of interest as defined elsewhere herein, may be performed by applying (such as pipetting) the compound of interest onto the ECM/mesothelial cell suspension as defined herein or embedding the compound of interest within the ECM/mesothelial cell suspension. The contacting is dependent on its formulation (e.g. whether using a 2D or 3D system). In such contacting step, the compound of interest should directly and/or indirectly target the mesothelial cells within the suspension for modulating ECM movement. The compound of interest may be added to the mesothelial cells of the suspension before performing step b) as defined herein or the compound of interest may be added to the mesothelial cells of the suspension after performing step b) as defined herein. Such method step c) can be performed either manually or automatically.

### Step d)

In the following, such ECM movement is then detected/recorded by any detection method as described herein comprising any imaging method suitable and as defined herein, followed by quantification using techniques such as fluorimetry and AI/machine learning as known in the prior art. The movement of the ECM which is detected can be towards or away from wherever the external stimulus (the compound of interest in some cases) was applied. Such determination step is performed in comparison to a control single cell suspension which is the same as defined above, thus also a mesothelial single cell suspension as defined elsewhere herein. However, said ECM of said control, which either said single cell suspension has been contacted with as defined above or which has been produced by said cells themselves under suitable conditions as defined herein, has been labelled, but said cells of said control has not been contacted with said compound of interest.

### EXAMPLES OF THE INVENTION

The following Examples illustrate the invention, but are not to be construed as limiting the scope of the invention.

### Material and Methods

### Patient Derived Tissue.

All tissues (PFA, ST) used in this study were obtained with properly informed consent of patients. All experimental procedures were performed in accordance with the Ethics committee vote number/Study protocol number: 333-10 Parent Proposal number: BA34/2018.

### Mouse Housing and Husbandry.

C57BL/6J mice where purchased from Jackson Laboratories or Charles River and bred and maintained in the Helmholtz Animal Facility in accordance with EU directive 2010/63. IFN-γ-R−/− mice on C57BL/6 background were originally obtained from the Jackson Laboratory (Bar Harbor, ME, USA) and subsequently bred and propagated under SPF conditions at the Helmholtz Zentrum München. Animals were housed in individual ventilated cages and animal housing rooms were maintained at constant temperature and humidity with a 12-h light cycle. Animals were supplied with water and chow ad libitum. All animal experiments were reviewed and approved by the Government of Upper Bavaria and registered under the project number ROB-55.2-2532.Vet_02-19-101 or ROB-55.2-2532.Vet_02-18-97 and conducted under strict governmental and international guidelines. This study is compliant with all relevant ethical regulations regarding animal research.

### In Vivo Matrix Fate Tracing.

The inventors generated a labelling solution by mixing 5 μl NHS-ester (25 mg/ml) with 5 μl of 100 mM pH 9.0 sodium bicarbonate buffer, combining with 40 μl PBS to a total volume of 50 μl. Labelling solution was applied intrapleurally (for lung and/or heart fibrosis model) under isoflurane anesthesia with a 30 G cannula. Abdominal labeling was performed by injecting 100 μl of labelling solution intra-peritoneal (for liver and/or kidney fibrosis model).

### Bleomycin Induced Pneumonia Model.

The oropharyngeal administration of bleomycin for the induction of pulmonary fibrosis is carried out in an antagonistic anesthesia in C57BL/6J mice of both sexes (6-8 weeks age). After the toe-pinch reflex is absent, the mouse is placed on the incisors of the upper jaw and thus kept in an upright position. The tongue is carefully fixed with is held to the side with tweezers and the nose of the animal is covered with tweezers. By keeping the nose closed, the mouse is forced to breathe through the mouth. With the help of a pipette, bleomycin is dissolved in a dosage of 2 units/kg KGW in 80 μl PBS carefully into the throat. As soon as the animal has inhaled the solution, it will be

Hot plate transferred (duration approx. 30 to 60 seconds). After antagonization animals were housed for 14 days. Nintedanib was added 1 hour before bleomycin installation and every other day intra peritoneal 10 μM.

### Pharmacologic Regime.

Pirfenidone (0.07 mg/kg), Nintedanib (0.21 mg/kg) and Cathepsin Inhibitor B (0.15 mg/kg) were injected 1 hour before bleomycin installation and every other day intra peritoneal in a volume of 100 μl in physiological saline solution.

### Herpes Induced Pneumonia Model.

Mice were housed in individually ventilated cages during the MHV-68 infection period. Mice were infected intranasally (i.n.) with 5×10*4 plaque forming units of MHV-68 diluted in PBS in a total volume of 30 μl. Prior to i.n. infection, mice were anesthetized with medetomidine-midazolam-fentanyl and NHS-FITC was applied to label organ surfaces. At the predetermined time points, mice were sacrificed by cervical dislocation and tissues were processed for subsequent experiments.

### Recombinant TGF Model.

100 ng of recombinant TGF-β was applied intra pleural under isoflurane anesthesia with a 30 G cannula.

### Plasmid Construction.

For construction of plasmids utilized for AAV production, cDNA was generated from mRNA extracted from C57BL/6 mice tissue utilizing SuperScript IV Reverse Transcriptase (Life Technologies). Produced cDNA was utilized as template for PCRs amplifying coding sequences of murine TGFβ (TGFb), murine cathepsin B (CTSB) and murine cystatin A (CSTA), murine Dcn, murine Sparc, murine mgp, murine plac8, murine lgals1 and murine ifi2712a using KOD Hot Start DNA Polymerase (Merck Millipore). Analog, the dominant negative mutant of murine TGFβRII (TGFbRII-DN) according to the published human version was produced. Flag-tagged murine collagen 1a2 (Col1a2-Flag) was created based on plasmid eGFP-proα2(1) (gifted by Sergey Leikin; Addgene plasmid #119826; http://n2t.net/addgene:119826; RRID:Addgene_119826) replacing eGFP with the Flag-tag coding sequence. PCR products were cloned into pAAV-Cp-SV40 pA, which was constructed (i) to mediate strong overexpression of an incorporated transgene (ii) utilizing well established components with minimal size (iii) to maximize the capacity for the transgenic sequence. The created expression cassette contains the immediate early promoter of the human Cytomegalovirus (CMV) including the respective enhancer (Cp), a consensus Kozak sequence and the polyadenylation signal sequence of the Simian Virus 40 (SV40 pA) flanked by AAV2 derived inverted terminal repeats (ITRs). This expression cassette was incorporated in between the two Inverted Terminal Repeats (ITRs) of the AAV serotype 2 essential for efficient generation and packaging of AAV vector genomes during production. For cloning the In-Fusion HD Cloning Plus (Takara Bio Inc.) was utilized and final plasmids were verified by sequencing (S. Omari et al. (2018) Proc. Natl. Acad. Sci. 115). For generation of capsid-modified AAV8RGD the plasmid pAAV2/8, a gift from James M. Wilson (Addgene plasmid #112864; http://n2t.net/addgene:112864; RRID:Addgene_112864), was modified by incorporation of peptide TGCDCRGDCFCG between amino acid 584 and 585 of VP1, similar to previously published AAV2 capsid modification. Final plasmid pAAV2/8RGD was verified by sequencing. Sequences are uploaded into the repository, details of the plasmid construction procedure can be obtained upon request.

### AAV Production.

Production and purification of AAV-preparations for AAV8RGD-TGFb, AAV8RGD-TGFbRII-DN, AAV8RGD-CTSB, AAV8RGD-CSTA, AAV8RGD-Col1a2-Flag, AAV8RGD-Dcn, AAV8RGD-Sparc, AAV8RGD-MGP, AAV8RGD-Lgals1, AAV8RGD-Plac8, and AAV8RGD-Ifi2712a was performed according to the AAVpro® Purification Kit Maxi (Takara Bio Inc.) protocol. In brief, 5×T225-flasks were triple-transfected with the plasmid pHelper from the kit AAVpro® Helper Free System (AAV6) (Takara Bio Inc.), the plasmid pAAV2/8RGD containing coding sequences of the AAV2 derived rep proteins and the modified AAV8 capsid proteins and the pAAV-Cp-SV40 pA derivate containing the AAV-genome with the respective transgene. 96 h post transfection cells were harvested and AAV vector particles released by breaking up the cells with 3× freeze-thaw cycles. Genomic DNA was digested with Cryonase cold-active nuclease and AAV vector particles separated from cell debris by filtration (0.45 μm filter). Finally, AAV particles were separated from low molecular contaminants utilizing 100 kDa size exclusion columns and concentrated. Titers of final AAV preparations were determined via qPCR utilizing the AAVpro Titration Kit (qPCR) V2 (Takara Bio Inc.).

### AAV Application in Mice.

For application of AAVs in mice, viral vector preparations were diluted with PBS (1×) to a final concentration of 6×10⁸ viral particles/μl. 50 μl of respective vector dilutions were used for intrapleural injections (total dose of 3×10¹⁰ viral particles) via 30 G canula.

### Ex Vivo Culture of Lung Biopsies.

C57BL/6J male mice (6-8 weeks age) used to study the movement of lung matrix. After organ withdrawal 4 mm biopsy punches of murine lungs were generated. To obtain ectopic labeling of matrix, the inventors generated a labelling solution by mixing NHS-ester 1:1 with 100 mM pH 9.0 sodium bicarbonate buffer. Sterile Whatman filter paper (Sigma Aldrich) biopsy punches where soaked in NHS-labelling solution, and locally placed on the lung biopsy surface. After one minute, the labelling punch was removed. Mouse lung biopsies were cocultured in the RPMI medium (10% FBS with 1% Pen/Strep and 0.1% AmB) consist of different sub types of immune cells (0.1×10⁶ cells/biopsy) isolated from the healthy and idiopathic pulmonary fibrosis human donors. Mouse lung biopsies with immune cells were then cultured in the ex vivo condition provided with 5% C02 at 37° C.

After 48 hours, mouse lung biopsies were fixed with the 4% formalin and incubated for overnight at 4° C. followed by PBS wash. Human lung tissues where obtained, labelled, and cultivated for 24 hours as described above.

### Tissue Preparation Histology.

Upon organ excision, organs were fixed overnight at 4° C. in 2% formaldehyde. The next day, fixed tissues were washed three times in Dulbecco's phosphate buffered saline (DPBS, GIBCO, #14190-094), and depending on the purpose, either embedded, frozen in optimal cutting temperature compound (Sakura, #4583) and stored at −20° C., or stored at 4° C. in PBS containing 0.2% gelatin (Sigma Aldrich, #G1393), 0.5% Triton X-100 (Sigma Aldrich, #X100) and 0.01% Thimerosal (Sigma Aldrich, #T8784) (PBS-GT). Fixed tissues were embedded in optimal cutting temperature (OCT) and cut with a Microm HM 525 (Thermo Scientific). In short, sections were fixed in ice-cold acetone for 5 min at −20° C., and then washed with PBS. Sections were then blocked for non-specific binding with 10% serum in PBS for 60 minutes at room temperature, and then incubated with primary antibody in blocking solution O/N at 4° C. The next day, following washing, sections were incubated in PBS with fluorescent secondary antibody, for 120 min at RT. Finally, sections were washed and incubated with Hoechst 33342 nucleic acid stain (Invitrogen, #H1399), washed in ddH₂O, mounted with Fluoromount-G® (Southern Biotech, #0100-01), and stored at 4° C. in the dark.

### Histology and Murine Ex Vivo Imaging.

Histological sections were imaged under a M205 FCA Stereomicroscope (Leica) and ZEISS Axiolmager Z2m (Carl Zeiss). Murine biopsy punches were imaged under a M205 FCA Stereomicroscope (Leica). Data was processed with Imaris 9.1.3 (Bitplane) and ImageJ (1.52i). Contrast and brightness were adjusted for better visibility. Thundering was performed with fluoromount and standard parameter settings for histology cuts.

### 3D Lightsheet Imaging.

Whole-mount samples were stained and cleared with a modified 3DISCO protocol. Samples were dehydrated in an ascending tetrahydrofuran (Sigma Aldrich, #186562) series (50%, 70%, 3×100%; 60 minutes each), and subsequently cleared in dichloromethane (Sigma Aldrich, #270997) for 30 min and eventually immersed in benzyl ether (Sigma Aldrich, #108014). Cleared samples were imaged whilst submerged in benzyl-ether with a light-sheet fluorescence microscope (LaVision BioTec). Whilst submerged in benzyl-ether, specimens were illuminated on two sides by a planar light-sheet using a white-light laser (SuperK Extreme EXW-9; NKT Photonics). Optical sections were recorded by moving the specimen chamber vertically at 5-mm steps through the laser light-sheet. Three-dimensional reconstructions were obtained using Imaris imaging software (v 9.1.3, Bitplane).

### 3D Multiphoton Imaging.

For multi-photon imaging, samples were embedded in a 4% NuSieve GTG agarose solution (Lonza, #50080). Imaging was performed using a 25× water-dipping objective (HC IRAPO L 25×/1.00 W) coupled to a tunable pulsed laser (Spectra Physics, Insight DS+). Multi-photon excited images were recorded with external, non-descanned hybrid photo detectors (HyDs). Following band pass (BP) filters were used for detection: HC 405/150 BP for Second Harmonic Generation (SHG) and an ET 525/50 BP for green channel Tiles were merged using Leica Application suite X (v 3.3.0, Leica) with smooth overlap blending and data were visualized with Imaris software (v 9.1.3, Bitplane).

### Image Quantification.

Matrix invasion was calculated using histological sections, quantifying FITC signal per area via ImageJ (1.52i). Quantification of immunolabeling was performed in randomly distributed ROIs/FOVs. Multiphoton images were analyzed using Fiji plugins Fraklac V. 2.5 and LocalThickness_V-4.0.2.

scRNA-Seq Analysis.

Single cell sequencing data of whole mouse lung lysates from a bleomycin time course experiment was re-analyzed regarding the Mesothelial cells (Strunz et al. (2020) Nat. Commun. 11). Differentially expressed genes across timepoints within the Mesothelial cells were identified, using the R packages splines and lmtest, as previously described ((Strunz et al. (2020) Nat. Commun. 11). Differentially expressed genes between PBS controls and bleomycin-induced lung fibrosis samples, were calculated using the al/Markers function of scanpy. These genes were compared with the differentially expressed genes between human ILD (Interstitial Lung Diseases) patients and controls within mesothelial cells, from an integrated human ILD lung cell atlas (C. H. Mayr et al. (2020). SSRN Electron. J., 1-30).

### Mass Spectrometry.

Tissues were snap frozen and ground using a tissue lyser (Qiagen). Pulverized tissues were resuspended in lysis buffer (20 mM Tris-HCl pH 7.5, 1% Triton X-100, 2% SDS, 100 mM NaCl, 1 mM sodium orthovanandate, 9.5 mM sodium fluoride, 10 mM sodium pyruvate, 10 mM beta-glycerophosphate), and supplemented with protease inhibitors (complete protease inhibitor cocktail, Pierce) and kept 10 min on ice. Samples were then sonicated and spun down for 5 minutes at 10,000 g. Supernatants were stored at −80° C. Protein concentrations were determined via BCA-Assay according to the manufacturer's protocol (Pierce).

Protein pulldown was as follows. Lysates were diluted with a pulldown buffer (20 mM Tris-HCl pH 7.5, 1% Triton X-100, 100 mM NaCl, supplemented with protease and phosphatase inhibitors) and incubated overnight with dynabeads (Thermo Fisher) according to the manufacturer's instructions at 4° C. on a rotator. The next day, the samples were each diluted twice with Wash Buffer 1 (pulldown buffer plus 2% SDS) and then with Wash Buffer 2 (pulldown buffer with reduced, 0.5% Triton X-100) and finally washed twice with Wash Buffer 3 (20 mM Tris-HCl pH 7.5 and 100 mM NaCl). Beads were then resuspended in Elution Buffer (20 mM Tris-HCl pH 7.5, 100 mM NaCl and 50 mM DTT) and incubated for 30 minutes at 37° C. Finally, the samples were boiled for 5 minutes at 98° C. and the supernatants were stored at −80° C. Samples were digested using a modified FASP procedure. After reduction and alkylation using DTT and IAA, the proteins were centrifuged on Microcon® centrifugal filters (Sartorius Vivacon 500 30 kDa), washed thrice with 8 M urea in 0.1 M Tris/HCl pH 8.5 and twice with 50 mM ammonium bicarbonate. The proteins on filters were digested for 2 hours at room temperature using 0.5 μg Lys-C (Wako Chemicals) and for 16 hours at 37° C. with 1 μg trypsin (Promega). Peptides were collected by centrifugation (10 min at 14000 g), acidified with 0.5% TFA and stored at −20° C. until measurements. The digested peptides were loaded automatically on a HPLC system (Thermo Fisher Scientific) equipped with a nano trap column (100 μm ID×2 cm, Acclaim PepMAP 100 C18, 5 μm, 100 Å/size, LC Packings, Thermo Fisher Scientific) in 95% buffer A (2% ACN, 0.1% formic acid (FA) in HPLC-grade water) and 5% buffer B (98% ACN, 0.1% FA in HPLC-grade water) flowing at 30 μl/min. After 5 min, the peptides were eluted and separated on the analytical column (nanoEase MZ HSS T3 Column, 100 Å, 1.8 μm, 75 μm×250 mm, Waters) for 105 minutes at 250 nl/min flow rate in a 3 to 40% non-linear acetonitrile gradient in 0.1% formic acid. The eluting peptides were analyzed online in a Q Exactive HF mass spectrometer (Thermo Fisher Scientific) coupled to the HPLC system with a nano spray ion source, operated in the data-dependent mode. MS spectra were recorded at a resolution of 60,000 and after each MS1 cycle, the 10 most abundant peptide ions were selected for fragmentation. Raw spectra from mouse samples were analyzed with Progenesis QI software (version 4.1, Nonlinear Dynamics, Waters) and searched against the SwissProt mouse database (16,872 sequences) with Mascot (Matrix Science, version 2.6.2) with the following search parameters: 10 ppm peptide mass tolerance and 0.02 Da fragment mass tolerance, two missed cleavages allowed, carbamidomethylation was set as fixed modification, camthiopropanoyl, methionine and proline oxidation were allowed as variable modifications. A Mascot-integrated decoy database search calculated an average false discovery of <5% when searches were performed with a mascot percolator score cut-off of 13 and a significance threshold p-value. Peptide assignments were re-imported into the Progenesis QI software and the abundances of all unique peptides allocated to each protein were summed and normalized. Raw spectra from human samples were analyzed with Proteome Discoverer 2.4 software (Thermo Fisher Scientific; version 2.4.1.15) via a database search (Sequest HT search engine) against SwissProt human database (20,237 sequences), considering full tryptic specificity, allowing for up to two missed tryptic cleavage sites, precursor mass tolerance 10 ppm, fragment mass tolerance 0.02 Da. Carbamidomethylation was set as fixed modification, camthiopropanoyl, methionine and proline oxidation were allowed as variable modifications. Percolator was used for validating peptide spectrum matches and peptides, accepting only the top-scoring hit for each spectrum, and satisfying the cutoff values for FDR <1%, and posterior error probability <0.05. The final list of proteins complied with the strict parsimony principle and contains the summed and normalized abundances of all qualifying peptides. Extracellular elements were identified through a database search against a matrix gene database. Gene ontology analysis was performed using EnrichR webtool.

mRNA Transfection.

Human mesothelial Met5A cells (200.000 cells) were transfected via lipofectamine (Life Technologies) with 2 μg CleanCap mCherry mRNA (tebu bio). Mice were intra plurally injected with 0.5 μg/g body weight using in-vivo-jetpei (Polyplus transfection) according to manufacturer's protocol (see then Example 5).

### Injury Models for Obtaining Liver Tissue and Peritoneal Areas.

Thirty minutes before surgery mice received a preemptive subcutaneous injection with Metamizole (200 mg/kg bw). Anesthesia was supplied by an intraperitoneal injection of a Medetomidin (500 μg/kg), Midazolam (5 mg/kg) and Fentanyl (50 μg/kg) cocktail, hereafter referred to as MMF. Monitoring anesthetic depth was assessed by toe reflex. Eyes were covered with Bepanthen-cream to avoid dehydration, and the abdomen was shaved and disinfected with betadine and sterile phosphate buffered saline (PBS). Animals were kept on their backs on a heating plate at 39° C. A midline laparotomy (1-1.5 cm) was performed through the skin and peritoneum. Four hooks, positioned around the incision and fixed to a retractor and magnetic base plate, allowed for clear access to the abdominal cavity and liver.

Local damage to the liver surface was induced via electroporation tweezers by applying 30V 50 ms pulses at 1 s intervals for 8 cycles. Before closure of the incision, Buprenorphine (0.1 mg/kg) was pipetted in the abdomen to allow for initial post-surgical analgesia. For long-term analgesia, Metamizole (Novalgin, 200 mg/kg) was provided through daily injections. The peritoneum and skin were closed with two separate 4-0 silk sutures (Ethicon). Upon closure of the incision, mice were woken up by antagonizing Medetomidin and Midazolam through a subcutaneous cocktail injection of Atipamezol (1 mg/kg) and Flumazenil (0.25 mg/kg). Mice were allowed to recover on a heating pad, after which they were single housed. Mice were sacrificed after indicated time points and liver tissue was obtained. In the peritoneal model, the surgical procedure was as described above, but the peritoneal areas were marked (see then Example 4).

### Results

### Example 1: Matrix Reservoirs that Irrigate Organs Originate from Surface Mesothelium (See Also Example 4)

As the matrix that moves into wounds is located directly below the pleural mesothelium, the inventors sought to formally demonstrate if the mobile matrix that scars in lungs indeed comes from mesothelial cells. To this end, they used AAV8-based system, to transduce a collagen 1-FLAG (Col1-FLAG) reporter tag specifically into mesothelium. In this system, transduced cells that de novo express Col1 transcripts, generate a Col1-FLAG fusion protein that incorporates in collagen helices, thereby revealing the tissue sources for Col1. Intrapleural administration of AAV8RGD-Col1-FLAG resulted in a robust and specific viral transduction of mesothelial cells (FIG. 1A), without any interstitial labeling. Lung surfaces were then labeled with NHS ester, to tag matrix pools as above, followed by administration of bleomycin in trachea. This resulted in a dramatic flow of NHS ester-positive fluid material (green) into the interstitium that was co-positive for Collagen1-FLAG reporter (yellow). Green+ yellow+ double-positive patches, indicating pre-existing and newly deposited matrix, were abundant around blood vessel adventitia and bronchioles, and they completely irrigated the lungs. This indicates that fibrosis that ends up accumulating in lungs originates in part from mesothelia-born pleural pools of matrix that are being constantly generated de novo after injury (FIG. 1B).

Having demonstrated the mesothelium-origin for the matrix in pulmonary fibrosis, the inventors examined mesothelial gene expression kinetics during bleomycin-induced pulmonary fibrosis. The inventors analyzed scRNA-Seq data sets of lung mesothelium from bleomycin treatment on day 3, 7, 10, 14, 21 and 28 post bleomycin installation (FIG. 1C). Mesothelial cells dynamically expressed multiple matrix proteins, after Bleomycin exposure, that was fibrosis stage-dependent. Healthy lung mesothelium (day 0) express collagens Col4a3, Col4a4, laminins and mucin family members, consistent with basement membrane maintenance and a lubricating non-fibrosis role for healthy mesothelium. Three days post-bleomycin, mesothelial cells increase levels of the pro fibrotic mediator TGFβ, and had high levels of various fibrillary collagens of type 1, 4, 5, 7, 12, and 14, indicating diverse fibrillary collagens accumulate in matrix reservoirs. At day seven post-bleomycin, mesothelial cells expressed additional collagen family sub members of type 1, 4, 6, 7, 14 and 27 as well as fibulins, secreted glycoproteins that become incorporated into extracellular protein fibers and play a role in cellular transformations. At day ten, a stage where irrigation initiates, the inventors found thiol proteases such as Cathepsin family members B, C, D, E, F, H, and S were dramatically upregulated. At more progressive stages post bleomycin, i.e. at day 28, mesothelial cells dramatically decreased collagen and protease expression levels, reverting matrix protein expression back to that seen in baseline homeostatic states. These data demonstrate that the dynamic shifts in accretion profiles by mesothelial cells mirrors fibrosis development.

### Example 2: Mesothelial TGFβ Alone Triggers Fibrosis

The inventors sought to characterize the signaling pathways by which immune cells trigger matrix invasion and fibrosis and we naturally sought to investigate the known chief proponent, TGFβ. They wondered whether the fibrotic cascade of immune cells might start with mesothelial TGFβ expression. Mouse lung biopsies were treated with granulocytes, followed by administering two TGFβ inhibitors, Repsox and LDN-212854, in separate experiments (FIG. 2A). Inhibition of TGFβ signaling in both chemical assays decreased phosphorylated SMAD (pSMAD), which is the major mediator of TGFβ-induced signaling. Furthermore, inhibiting TGFβ reduced matrix invasion, even in the presence of granulocytes. Moreover, adding granulocytes alone led to increased phosphorylated SMAD signaling in mesothelium. These findings indicate granulocytes trigger TGFβ expression in mesothelium to initiate matrix accumulation and irrigation.

To investigate whether mesothelial TGFβ expression alone, could irrigate lungs with transferred matrix and cause fibrosis, the inventors performed the murine ex vivo pleural matrix tracing assay, in the presence of recombinant TGFβ (rTGFβ, FIG. 2A). rTGFβ induced expansion and thickening of matrix pools on lung surfaces, accompanied by a massive influx of matrix into the interstitium. Next, the inventors investigated TGFβ signaling in fibrotic lungs in vivo. They detected massive phosphorylation of SMAD, in mesothelium from bleomycin-treated lungs that disappeared at the time of fibrosis resolution (FIG. 2B), and activated SMAD signaling in mesothelium coincided with thickening of the pleural matrix pool in vivo. Moreover, in the viral model there was massive pSMAD signaling in the pleural lining, and thickening of the pleural matrix pool (FIG. 2C). Next, the inventors investigated the involvement of mesothelial TGFβ in matrix movements and fibrosis progression, by specifically overexpressing TGFβ in the pleural mesothelium, in combination with NHS ester thereby labeling matrix pools in vivo. Intrapleural injection of recombinant TGFβ in animals activated robust phosphorylation of SMAD in mesothelium (FIG. 2D). This was followed by thickening of the pleural matrix pools within a week. Two weeks post TGFβ treatment, the lungs were completely filled with transferred matrix, which was accompanied by increased abundance of PDGFR+ myofibroblasts, interstitial fibrosis, significant weight loss and mortality (FIG. 2E-G). This suggests a stepwise process wherein mesothelial TGFβ induces a buildup of pleural matrix pools, which is subsequently released/liberated inwards to cause fibrosis.

To further corroborate if the TGFβ signaling responsible for matrix movement and fibrosis comes from mesothelium, we used a vector encoding an activated variant of TGF P, and stably expressed the activated variant of TGFβ in mesothelium (FIG. 2H). In the absence of injury, over-expression of the activated form of TGFβ in mesothelium resulted in matrix buildup on pleural surfaces, accompanied by massive influx of matrix material, increased amounts of PDGFR+ myofibroblasts, interstitial fibrosis, weight loss, and mortality (FIG. 2I-K), This demonstrates that mesothelial TGFβ alone liberates pleural matrix pools inwards to drive fibrosis.

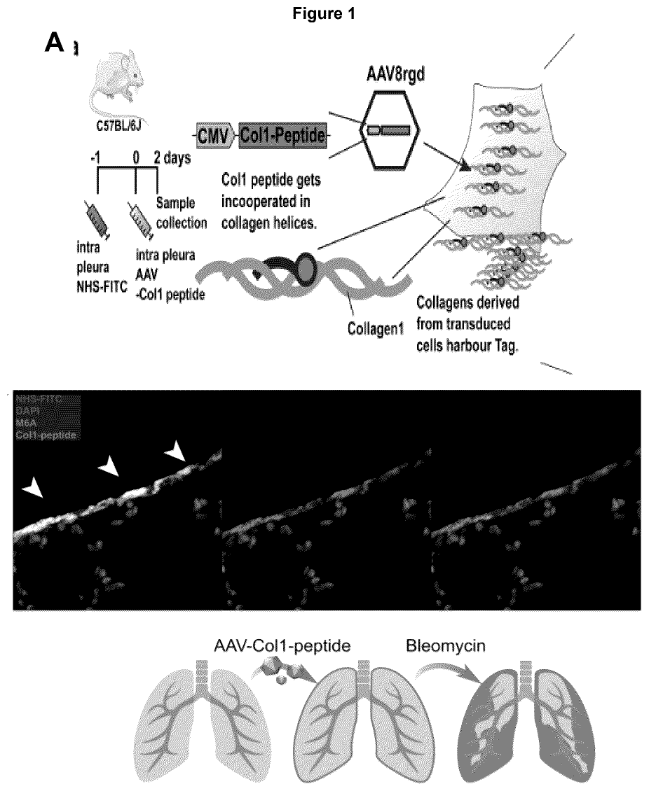

Next, the inventors investigated if mesothelial-specific TGFβ knockdown could inhibit fibrosis in the presence of chemical induced injury. To this aim, they over expressed a dominant-negative TGFβ receptor-dead mutant that intercepts incoming TGFβ and thus prevents its signal transduction in cells, exclusively in the mesothelial cells of the visceral pleura (FIG. 2L). Next, they investigated the effects of mesothelial TGFβ inhibition on bleomycin-induced fibrosis (FIG. 2M) by injecting bleomycin in the trachea of these mice. Indeed, inhibiting mesothelial TGFβ signaling alone completely blocked matrix buildup, and matrix invasion, stunted phosphorylated SMAD signaling, PDGFR+ myofibroblasts, fibrosis progression, and blocked bleomycin-induced mortality, culminating in a 100% survival rate (FIG. 2M-0).

### Example 3: TGFβ Causes Matrix Irrigation and Fibrosis Through Cathepsin B

As mesothelial TGFβ expands matrix pools on pleural surfaces, the inventors revisited the scRNAseq data for candidate mediators of matrix liberation and influx into the organs. To establish a further link to pulmonary fibrosis, the inventors compared the gene expression of collagens and thiol proteases in mesothelial cells in the bleomycin model with that of human interstitial lung disease patients (FIG. 3A). Their rationale was that since the pleural matrix pool is a fibrous network of proteins, its liberation inwards must be mediated by a protease. By analyzing the transcriptomes of mouse and human fibrosis they found that mesothelial cells upregulate cathepsins, and their inhibitory counterparts, the cystatins, in response to bleomycin and in fibrotic human lungs. Specifically, Cathepsin B started to peak at 10 days after bleomycin treatment, correlating with the increased abundance of transferred matrix within the interstitium, and Cathepsin B proteins were absent at 45 days, the time that fibroses resolve (FIG. 3B). Moreover, their viral infection model triggered a similar increase in Cathepsin B expression in mesothelium covering lungs (FIG. 3C) and liver and kidneys (FIG. 5).

To study if Cathepsin B is downstream of TGFβ and required for it causing matrix invasion, the inventors used AAV viral delivery to overexpress it specifically in mesothelium. Indeed, TGFβ overexpression in mesothelium, induced Cathepsin B expression, followed by massive inward movements of matrix in the absence of any chemical or viral injury (FIGS. 3D and 3E). However, in the presence of bleomycin, inhibiting mesothelial TGFβ with a dominant-negative mutant form, dramatically reduced the levels of Cathepsin B expression and blocked the release of matrix (FIG. 3F).

To prove the feasibility of specific pharmacologic rescue with Cathepsin B, the inventors gave mice NHS ester to label pleural surfaces as before, then injected bleomycin in trachea, followed by treatments with, Cathepsin B inhibitor, Z-FA-FMK that irreversibly blocks the active center of Cathepsin B. As positive controls they used Nintedanib and Pirfenidone, the only two anti-fibrotic drugs on the market that have been approved for pulmonary fibrosis, both of which have anti-inflammatory and anti-fibrosis activities that impede disease progression. In the presence of bleomycin, pharmacologic intervention of Cathepsin B completely prevented matrix invasion into lungs and fibrosis development and abrogated bleomycin-induced mortality (FIG. 6 A-F), increasing survival rates of lung injured animals to 100%. These rates were comparable to Nintenanib and Pirfenidone. These rates were comparable to Nintenanib and Pirfenidone. Since mesothelial cells of fibrotic lung tissue produce elevated levels of thiol proteases, we tested our inhibitor regime with human lung tissue and heart (FIG. 6G). In order to prove that mesothelial Cathepsin B, downstream of TGFβ, drives fibrosis, the inventors overexpressed AAV vector-based constructs of Cathepsin B and Cystatin A, a direct inhibitor that binds and blocks Cathepsin B protease (FIG. 3G). Both Cathepsin B and Cystatin A constructs were stably expressed in lung mesothelial cells, followed by pleural injection of NHS ester to label matrix pools, and with bleomycin-treatment in mice (FIG. 3H). Overexpression of mesothelial Cathepsin B alone led to a dramatic increase in matrix influx and mortality as compared to control vector-treated animals. Conversely, suppression of mesothelial Cathepsin B by overexpressing Cystatin A stopped all matrix movements, even at the peak of fibrosis at 14 days post-bleomycin, and completely prevented bleomycin-induced mortality. These mechanistic findings directly link mesothelial TGFβ with Cathepsin B as driving matrix movements, fibrosis and mortality (FIG. 3I). The inventors conclude that mesothelial TGFβ expression builds up matrix pools, which are then released in a process catalyzed by Cathepsin B activation, leading to matrix flux and organ fibrosis (FIG. 4).

In summary, the inventors demonstrate that inflammation triggers a TGFβ:Cathepsin B signaling cascade in mesothelium that both expands pleural matrix pools and liberates proteinaceous material from pleural pools to irrigate lungs with scar tissue, causing fibrosis. Both Nintedanib and Pirfenidone exert anti-fibrosis effects by inhibiting matrix movements, and their findings indicate that pharmacological inhibition of mesothelial Cathepsin B with Cystatin A may serve as a more specific and effective treatment to combat organ fibrosis and improve disease progression. Matrix irrigation is likely a general principle of tissue/organ injury with potential clinical ramifications to many human fibrotic conditions.

### Example 4: Transferred Fibrotic Matrix Comes from Mesothelium

As the matrix that moves into wounds is located directly below the mesothelium, the inventors next sought to formally ascertain if the matrix from organs different to lung (see Example 1) that forms in wounds indeed comes from mesothelial cells. To identify the cellular sources of the mobile matrix, the inventors used a native collagen 1 binding protein reporter (CNA35) that was fused to mCherry fluorescent protein (FIG. 7A). In this system, transduced cells alone generate Col1CNA35-mCherry fusion protein that incorporates in collagen helices, thereby revealing the specific tissue sources for Col1 and enabling real-time visualization and quantification of collagen deposition in live tissues.

To zero-in on the mesothelial source of transferred matrix, the inventors virally transfected an area of organ surface with CNA35-mCherry thus tagging newly synthesized collagen produced by mesothelium alone. After five days the inventors then labelled the matrix with NHS-FITC, and subjected remote surfaces that lacked CNA35-mCherry or NHS-FITC tag, to liver and peritoneal wounds. Light-sheet images (FIG. 7B) and histology sections (FIG. 7C) of the wound areas revealed extensive accumulation of CNA35-mCherry and NHS-FITC double-positive matrix that transferred into wounds. This translocated material made up 70% and 80% of total collagen of peritoneal and liver wounds, respectively (FIG. 7D). Immunochemistry staining of the original labelling site also showed that after one week, significant amounts of mesothelial cells exhibited active TGF beta signalling, suggesting active mesothelial cells replenishing fluid matrix pools (FIG. 7E).

### Example 5: mRNA Transfection

Commercial mCherry mRNA was successful introduced into human mesothelial Met5A cells using lipofectamine, showing massive mCherry expression 24 hours post transfection (FIG. 8A). This demonstrated that mRNA-mediated reprogramming of mesothelial cells is an attractive method.

Next, the inventors used PolyPlus-mediated in vivo transfection of mRNA via the intra pleural injection. Indeed, within 24 hours of injecting the transfection mix, the inventors were able to detect large sections of lung mesothelium that were mCherry positive (FIG. 8B).

### Example 6: Verification of Further Candidate Genes Important for ECM Movement Via Ex Vivo Model of PCLS

Precision-cut lung slices (PCLS) were obtained from adult mouse lungs. Adult C57BL/6J mice were anesthetized using Ketamine/Xylazine (100 mg/kg and 10 mg/kg in 0.9% NaCl) by intraperitoneal injection. The anterior chest wall was excised and trachea was carefully exposed and lungs were perfused with saline. A tiny opening was made in the anterior wall of the trachea just below the cricoid cartilage. A rigid metallic cannula (P14) was carefully inserted through the trachea up to a millimeter above the bifurcation of the principal bronchi and fixed in place by suture. After cannulation, the lungs were inflated with 37° C. 1.5% low-melting-point agarose (Sigma; Cat. No. A9414) prepared with 1×DMEM medium (Life Technologies; Cat. No. 31966-021). Agarose was injected to inflate both lungs keeping them in situ within the chest cavity at volume that enabled lungs to be fully inflated without hyper- or sub-optimal inflation (1 ml agarose). After inflation, agarose was solidified by applying ice to the chest cavity for 1 in. Subsequently, the lungs were excised from the body along with heart and trachea and immersed in ice-cold serum-free DMEM. Lungs were after incubated at 37° C. with the different AAVs in DMEM medium, washed with PBS 3 times for 10 min and surfaces labeled using NHS-FITC. In order to ensure similar sized slices only left lung lobes were used and cut transversely at 300 μm using a vibratome (Zeiss Hyrax V50 vibratome) in cold DMEM medium. Slices obtained were placed in a 24-well plate in ice-cold DMEM for all experiments. PCLS were then washed twice with warm DMEM to remove excess agarose from the tissue and incubated at 37° C. Bleomycin was added into medium (0.1 U/mL) for the corresponding groups and slices were incubated at 37° C. in presence of 5% CO₂ and 95% air. Media was supplemented with 1% penicillin-streptomycin (Life Technologies; Cat. No. 15140122) and media was changed on the day 3. The ECM movement was assessed using M205 FCA Stereomicroscope (Leica) at day 3 and day 5 (FIG. 9).

### Example 7: Discovery of New Essential Promoters

With regard to FIGS. 10 and 11 (preparation as in Example 6) in the process the displayed genes (crip1, lgals1, mgp, saa3 and sepp1) were overexpressed. Immunostainings of mouse lungs were imaged at different time points (day 5, 10 and 14, see FIG. 10) after bleomycin administration. The amount of signal/proteins per time point was used to tell when which protein was expressed, and thus when which promoter is active. These data clearly demonstrate which promoters are interesting for application.

### Discussion

Injured and fibrotic organs, such as pulmonary fibrosis, have been assumed to form scars primarily from connective tissue that is newly synthesized by fibroblasts. The data presented here paint a new picture by revealing that pre-made pleural connective tissues translocate from organ surfaces, irrigating interstitial and vascular spaces to precede and form an essential prerequisite for fibrosis. This new scar-cement brings fibrous building blocks as well as the corresponding enzymes to mature the tissue into a fibrotic scar, on-site. Thus, lungs trigger scarring and fibrosis primarily by translocating pre-existing connective tissue, followed by fibroblasts then. The inventors show that the transferred scar mix comes from pleural and visceral mesothelial cells that are activated by TGFβ and subsequently liberate this proteinaceous material through Cathepsin B (FIG. 3). Moreover, the inventors reveal fibrosis can occur independent of immune cells once TGFβ:Cathepsin B signaling cascade is activated in mesothelium, but also that immune cells activate mesothelium thereby initiation matrix build up and invasion inwards to cause organ fibrosis. Although the results demonstrate the mesothelial matrix is an essential prerequisite for fibrotic scarring, fibroblasts deposit matrix, to further contribute to scarring. However, this can only be at most part of a secondary response to the initial matrix irrigation. Furthermore, the experiments demonstrate that where there is no matrix irrigation, fibroblasts remain dormant and refrain from depositing any matrix, even after exposure to bleomycin or after lung injury. The inventors not only demonstrate the initiating source of fibrosis as the mesothelial coverings of organs but also show that immune cells and inflammation stimulate mesothelia to trigger a TGF signaling cascade that works through Cathepsin protease. Progression of matrix irrigation and fibrosis, as the inventors show, can be induced by local mesothelial Cathepsin B or blocked with local mesothelial Cystatin A treatments, without requiring long term inflammatory suppression, as is the case with Pirfenidone and Nintedanib. The TGFβ:Cathepsin signaling pathway in mesothelial cells therefore offers multiple hubs to protect against Covid linked pneumonia and fibrosis in any organ. Taken together, these findings with chemical and viral injury models, reveal immune-mesothelium crosstalk.

### ITEMS

1. A compound for use in a method for the modulation of movement of extracellular matrix (ECM) produced by mesothelial cells forming the surface of an internal organ, towards a site of injury of said organ of a subject suffering from or being at a risk of an injury of said organ.
2. The compound for the use of item 1, wherein modulation comprises that said compound is capable of specifically targeting mesothelial cells.
3. The compound for the use of item 1 or 2, wherein modulation is inhibition or promotion.
4. The compound for the use of any one of the preceding items, wherein said compound is a transcription construct encoding a gene involved in the modulation of movement of ECM produced by mesothelial cells.
5. The compound for the use of item 4, wherein said gene is selected from the group consisting of csta, tgfb, tgfbr2, ctsb, aebp1, col1a1, adamTs1, dcn, sparc, timp1, c1, c2, c3, c4, saa3, hsf1, and dtr.
6. The compound for the use of item 4 or 5, wherein the transcription construct comprises DNA, preferably wherein if the transcription construct is a DNA construct, said construct further comprises a mesothelium specific control element and/or promoter element and/or enhancer element and/or wherein if the transcription construct is a DNA construct, said construct further comprises a RNA or protein target sequence.
7. The compound for the use of item 4 or 5, wherein the transcription construct comprises RNA, preferably wherein if the transcription construct is a RNA construct, said construct further comprises a RNA or protein target sequence.
8. The compound for the use of any one of items 1-7, wherein the compound is an agonist or antagonist of a mesothelium specific receptor, preferably wherein the agonist or antagonist is selected from an antibody, a siRNA, a nucleic acid, an aptamer, a peptide, a protein, a lipid or a small organic molecule.
9. The compound for the use of item 8, wherein the mesothelium specific receptor is selected from the group consisting of MSLN1, GPM6A, PDPN, TGF-β receptor, LTB4 receptor BLT2, Podoplanin, and Procr.
10. The compound for the use of any one of the preceding items, wherein the compound is administered via injection or infusion, preferably wherein the administration is performed intravenously, intrathecally, intraperitoneally, intrapleurally, via pericardiocentesis or via the lymphatic system.
11. The compound for the use of item 10, wherein the compound is administered via a viral vector, a liposome, a transfection reagent, an extracellular vesicle or directly, preferably wherein the viral vector is an adeno-associated virus (AAV) vector and/or an adeno-virus (AV) vector.
12. The compound for the use of any one of the preceding items, wherein said internal organ is any one of a lung, a kidney, a heart, a liver, a stomach, a bladder, a brain, a peritoneum, an uterus, a spleen, a pancreas or an intestine.
13. The compound for the use of any one of the preceding items, wherein if modulation is inhibition, said injury of said organ is associated with a chronic wound or wherein if modulation is promotion, said injury of said organ is associated with a fibroproliferative disease.
14. A compound for use in an in vivo screening method for identifying a modulator of the movement of extracellular matrix (ECM) produced by mesothelial cells towards a site of injury of an internal organ of a subject, the method comprising
   a) contacting ECM of an internal organ of a subject with a label;
   b) introducing to said organ an injury;
   c) contacting mesothelial cells, which form the surface of said organ with a compound of interest;
   d) determining whether said compound of interest modulates movement of ECM towards a site of injury of said organ using a detection method in comparison to a control subject having ECM of said organ labelled, but not having mesothelial cells of said organ contacted with said compound of interest,
wherein modulation of the movement of ECM towards said site of injury of said organ is indicative for said compound of interest to be a modulator of said ECM movement and wherein step b) and step c) can be switched.
15. An in vitro screening method for identifying a modulator of the movement of extracellular matrix (ECM) towards an external stimulus in a single cell suspension derived from the mesothelium, the method comprising
   a) contacting a single cell suspension derived from the mesothelium with an already labeled ECM or placing a single cell suspension derived from the mesothelium under suitable conditions which allow said cells to produce ECM and then contacting of said produced ECM with a label;
   b) exposing said single cells to an external stimulus;
   c) contacting said single cells with a compound of interest;
   d) determining whether said compound of interest modulates ECM movement towards said external stimulus using a detection method in comparison to a control single cell suspension, wherein said ECM has been labeled, but said single cells not contacted with said compound of interest,
wherein modulation of the movement of ECM towards said external stimulus is indicative for said compound of interest to be a modulator of said ECM movement and wherein step b) and step c) can be switched.

1. A compound for use in a method for the modulation of movement of extracellular matrix (ECM) produced by mesothelial cells forming the surface of an internal organ, towards a site of injury of said organ of a subject suffering from or being at a risk of an injury of said organ, wherein modulation is inhibition or promotion.

2. The compound for the use of claim 1, wherein modulation comprises that said compound is capable of specifically targeting mesothelial cells.

3. The compound for the use of any one of the preceding claims, wherein said compound is a transcription construct encoding a gene involved in the modulation of movement of ECM produced by mesothelial cells.

4. The compound for the use of claim 3, wherein said gene is selected from the group consisting of csta, tgfb, tgfbr2, ctsb, aebp1, col1a1, adamTs1, dcn, sparc, timp1, c1, c2, c3, c4, saa3, hsf1, and dtr, or a combination thereof.

5. The compound for the use of claim 3 or 4, wherein said gene is selected from the group consisting of mgp, crip1, plac8, lgals1, and ifi2712a, or a combination thereof.

6. The compound for the use of any one of claims 3-5, wherein the transcription construct comprises DNA, preferably wherein if the transcription construct is a DNA construct, said construct further comprises a mesothelium specific control element and/or promoter element and/or enhancer element and/or wherein if the transcription construct is a DNA construct, said construct further comprises a RNA or protein target sequence.

7. The compound for the use of claim 6, wherein the mesothelium specific promoter element is any one of a CRIP1, LGALS1, MGP, SAA3 or a SEPP1 promoter, preferably CRIP1.

8. The compound for the use of any one of claims 3-5, wherein the transcription construct comprises RNA, preferably wherein if the transcription construct is a RNA construct, said construct further comprises a RNA or protein target sequence.

9. The compound for the use of any one of claims 1-8, wherein the compound is an agonist or antagonist of a mesothelium specific receptor, preferably wherein the agonist or antagonist is selected from an antibody, a siRNA, a nucleic acid, an aptamer, a peptide, a protein, a lipid or a small organic molecule.

10. The compound for the use of claim 9, wherein the mesothelium specific receptor is selected from the group consisting of MSLN1, GPM6A, PDPN, TGF-β receptor, LTB4 receptor BLT2, Podoplanin, and Procr.

11. The compound for the use of any one of the preceding claims, wherein the compound is administered via injection or infusion, preferably wherein the administration is performed intravenously, intrathecally, intraperitoneally, intrapleurally, via pericardiocentesis or via the lymphatic system.

12. The compound for the use of claim 11, wherein the compound is administered via a viral vector, a liposome, a transfection reagent, an extracellular vesicle or directly, preferably wherein the viral vector is an adeno-associated virus (AAV) vector and/or an adeno-virus (AV) vector.

13. The compound for the use of any one of the preceding claims, wherein said internal organ is any one of a lung, a kidney, a heart, a liver, a stomach, a bladder, a brain, a peritoneum, an uterus, a spleen, a pancreas or an intestine.

14. The compound for the use of any one of the preceding claims, wherein if modulation is inhibition, said injury of said organ is associated with a chronic wound or wherein if modulation is promotion, said injury of said organ is associated with a fibroproliferative disease.

15. A compound for use in an in vivo screening method for identifying a modulator of the movement of extracellular matrix (ECM) produced by mesothelial cells towards a site of injury of an internal organ of a subject, the method comprising
a) contacting ECM of an internal organ of a subject with a label;
b) introducing to said organ an injury;
c) contacting mesothelial cells, which form the surface of said organ with a compound of interest;
d) determining whether said compound of interest modulates movement of ECM towards a site of injury of said organ using a detection method in comparison to a control subject having ECM of said organ labelled, but not having mesothelial cells of said organ contacted with said compound of interest,
wherein modulation of the movement of ECM towards said site of injury of said organ is indicative for said compound of interest to be a modulator of said ECM movement and wherein step b) and step c) can be switched.

16. An in vitro screening method for identifying a modulator of the movement of extracellular matrix (ECM) towards an external stimulus in a single cell suspension derived from the mesothelium, the method comprising
a) contacting a single cell suspension derived from the mesothelium with an already labeled ECM or placing a single cell suspension derived from the mesothelium under suitable conditions which allow said cells to produce ECM and then contacting of said produced ECM with a label;
b) exposing said single cells to an external stimulus;
c) contacting said single cells with a compound of interest;
d) determining whether said compound of interest modulates ECM movement towards said external stimulus using a detection method in comparison to a control single cell suspension, wherein said ECM has been labeled, but said single cells not contacted with said compound of interest,
wherein modulation of the movement of ECM towards said external stimulus is indicative for said compound of interest to be a modulator of said ECM movement and wherein step b) and step c) can be switched.


SEQUENCE LISTING (1 sequence)

SEQ ID NO: 1 - protein, 12 aa, synthetic construct
  REGION 1..12: note = peptide sequence
TGCDCRGDCFCG